SEQUENCE LISTING (38 sequences)

SEQ ID NO: 1 - DNA, 268 bp, Homo sapiens
  source 1..268: mol_type = unassigned DNA
tcggaccgcggcaactacacctgcgtcgtggagaacaagtttggcagcatccggcagacgtacacgctggacgtgctggagtgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctca

SEQ ID NO: 2 - DNA, 378 bp, Homo sapiens
  source 1..378: mol_type = unassigned DNA
gaccgcggcaactacacctgcgtcgtggagaacaagtttggcagcatccggcagacgtacacgctggacgtgctgggtgagggccctggggcggcgcgggggtgggggcggcagtggcggtggtggtgagggagggggtggcccctgagcgtcatctgcccccacagagcgctgcccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaaggtgggccaccgtgtgcacgt

SEQ ID NO: 3 - DNA, 234 bp, Homo sapiens
  source 1..234: mol_type = unassigned DNA
gcgggcaattctattgggttttctcatcactctgcgtggctggtggtgctgccagccgaggaggagctggtggaggctgacgaggcgtgcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccg

SEQ ID NO: 4 - DNA, 301 bp, Homo sapiens
  source 1..301: mol_type = unassigned DNA
ctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtgtgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagc

SEQ ID NO: 5 - DNA, 21 bp, synthetic construct
  source 1..21: mol_type = other DNA
gacctggaccgtgtccttacc

SEQ ID NO: 6 - DNA, 21 bp, synthetic construct
  source 1..21: mol_type = other DNA
cttccccagttccaggttctt

SEQ ID NO: 7 - DNA, 20 bp, synthetic construct
  source 1..20: mol_type = other DNA
aggacctggaccgtgtcctt

SEQ ID NO: 8 - DNA, 20 bp, synthetic construct
  source 1..20: mol_type = other DNA
tataggtccggtggacaggg

SEQ ID NO: 9 - DNA, 20 bp, synthetic construct
  source 1..20: mol_type = other DNA
ctggaccgtgtccttaccgt

SEQ ID NO: 10 - DNA, 20 bp, synthetic construct
  source 1..20: mol_type = other DNA
gcagcccaggattgaactgt

SEQ ID NO: 11 - DNA, 23 bp, synthetic construct
  source 1..23: mol_type = other DNA
tggatcgaattctcactctcaca

SEQ ID NO: 12 - DNA, 21 bp, synthetic construct
  source 1..21: mol_type = other DNA
gccaagcaatctgcgtatttg

SEQ ID NO: 13 - DNA, 23 bp, synthetic construct
  source 1..23: mol_type = other DNA
gctcttcaatacagccctgatca

SEQ ID NO: 14 - DNA, 24 bp, synthetic construct
  source 1..24: mol_type = other DNA
acttggatcgaattctcactctca

SEQ ID NO: 15 - DNA, 23 bp, synthetic construct
  source 1..23: mol_type = other DNA
tggatcgaattctcactctcaca

SEQ ID NO: 16 - DNA, 25 bp, synthetic construct
  source 1..25: mol_type = other DNA
gcaaagcctgaattttcttgaataa

SEQ ID NO: 17 - DNA, 18 bp, synthetic construct
  source 1..18: mol_type = other DNA
gcatccggcagacgtaca

SEQ ID NO: 18 - DNA, 15 bp, synthetic construct
  source 1..15: mol_type = other DNA
ccccgcctgcaggat

SEQ ID NO: 19 - DNA, 18 bp, synthetic construct
  source 1..18: mol_type = other DNA
gcatccggcagacgtaca

SEQ ID NO: 20 - DNA, 15 bp, synthetic construct
  source 1..15: mol_type = other DNA
ccccgcctgcaggat

SEQ ID NO: 21 - DNA, 19 bp, synthetic construct
  source 1..19: mol_type = other DNA
aggagctggtggaggctga

SEQ ID NO: 22 - DNA, 19 bp, synthetic construct
  source 1..19: mol_type = other DNA
ccgtagctgaggatgcctg

SEQ ID NO: 23 - DNA, 18 bp, synthetic construct
  source 1..18: mol_type = other DNA
ctggtggaggctgacgag

SEQ ID NO: 24 - DNA, 16 bp, synthetic construct
  source 1..16: mol_type = other DNA
agcccaccccgtagct

SEQ ID NO: 25 - DNA, 21 bp, synthetic construct
  source 1..21: mol_type = other DNA
gtcgtggagaacaagtttggc

SEQ ID NO: 26 - DNA, 17 bp, synthetic construct
  source 1..17: mol_type = other DNA
gtctggttggccggcag

SEQ ID NO: 27 - DNA, 21 bp, synthetic construct
  source 1..21: mol_type = other DNA
gtcgtggagaacaagtttggc

SEQ ID NO: 28 - DNA, 17 bp, synthetic construct
  source 1..17: mol_type = other DNA
gtctggttggccggcag

SEQ ID NO: 29 - DNA, 19 bp, synthetic construct
  source 1..19: mol_type = other DNA
aggagctggtggaggctga

SEQ ID NO: 30 - DNA, 19 bp, synthetic construct
  source 1..19: mol_type = other DNA
ccgtagctgaggatgcctg

SEQ ID NO: 31 - DNA, 16 bp, synthetic construct
  source 1..16: mol_type = other DNA
gacgaggcgggcagtg

SEQ ID NO: 32 - DNA, 19 bp, synthetic construct
  source 1..19: mol_type = other DNA
gaagaagcccaccccgtag

SEQ ID NO: 33 - DNA, 2850 bp, synthetic construct
  source 1..2850: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 34 - DNA, 2955 bp, synthetic construct
  source 1..2955: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgtgccaggcccacccccaggtgttcccgcgcctgggggcccacccctgtccaccggacctatagtggacctgctccagtacagccagaaggacctggatgcagtggtaaaggcgacacaggaggagaaccgggagctgaggagcaggtgtgaggagctccacgggaagaacctggaactggggaagatcatggacaggttcgaagaggttgtgtaccaggccatggaggaagttcagaagcagaaggaactttccaaagctgaaatccagaaagttctaaaagaaaaagaccaacttaccacagatctgaactccatggagaagtccttctccgacctcttcaagcgttttgagaaacagaaagaggtgatcgagggctaccgcaagaacgaagagtcactgaagaagtgcgtggaggattacctggcaaggatcacccaggagggccagaggtaccaagccctgaaggcccacgcggaggagaagctgcagctggcaaacgaggagatcgcccaggtccggagcaaggcccaggcggaagcgttggccctccaggccagcctgaggaaggagcagatgcgcatccagtcgctggagaagacagtggagcagaagactaaagagaacgaggagctgaccaggatctgcgacgacctcatctccaagatggagaagatctga

SEQ ID NO: 35 - DNA, 3765 bp, synthetic construct
  source 1..3765: mol_type = other DNA
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacaatgttatggaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaactgaactgggacatgtcctcatagagatttcaagtacccacaagaaactcaacgagagtcttgatgaaaattttaaaaaattccacaaagagattatccatgagctggagaagaagatagaacttgacgtgaaatatatgaacgcaactctaaaaagataccaaacagaacacaagaataaattagagtctttggagaaatcccaagctgagttgaagaagatcagaaggaaaagccaaggaagccgaaacgcactcaaatatgaacacaaagaaattgagtatgtggagaccgttacttctcgtcagagtgaaatccagaaattcattgcagatggttgcaaagaggctctgcttgaagagaagaggcgcttctgctttctggttgataagcactgtggctttgcaaaccacatacattattatcacttacagtctgcagaactactgaattccaagctgcctcggtggcaggagacctgtgttgatgccatcaaagtgccagagaaaatcatgaatatgatcgaagaaataaagaccccagcctctacccccgtgtctggaactcctcaggcttcacccatgatcgagagaagcaatgtggttaggaaagattacgacaccctttctaaatgctcaccaaagatgccccccgctccttcaggcagagcatataccagtcccttgatcgatatgtttaataacccagccacggctgccccgaattcacaaagggtaaataattcaacaggtacttccgaagatcccagtttacagcgatcagtttcggttgcaacgggactgaacatgatgaagaagcagaaagtgaagaccatcttcccgcacactgcgggctccaacaagaccttactcagctttgcacagggagatgtcatcacgctgctcatccccgaggagaaggatggctggctctatggagaacacgacgtgtccaaggcgaggggttggttcccgtcgtcgtacacgaagttgctggaagaaaatgagacagaagcagtgaccgtgcccacgccaagccccacaccagtgagaagcatcagcaccgtgaacttgtctgagaatagcagtgttgtcatccccccacccgactacttggaatgcttgtccatgggggcagctgccgacaggagagcagattcggccaggacgacatccacctttaaggccccagcgtccaagcccgagaccgcggctcctaacgatgccaacgggactgcaaagccgccttttctcagcggagaaaacccctttgccactgtgaaactccgcccgactgtgacgaatgatcgctcggcacccatcattcgatga

SEQ ID NO: 36 - DNA, 4989 bp, synthetic construct
  source 1..4989: mol_type = other DNA
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatcatggaggaaacaaatacgcagattgcttggccatcaaaactgaagatcggagccaaatccaagaaagatccccatattaaggtttctggaaagaaagaagatgttaaagaagccaaggaaatgatcatgtctgtcttagacacaaaaagcaatcgagtcacactgaagatggatgtttcacatacagaacattcacatgtaatcggcaaaggtggcaacaatattaaaaaagtgatggaagaaaccggatgccatatccactttccagattccaacaggaataaccaagcagaaaaaagcaaccaggtatctatagcgggacaaccagcaggagtagaatctgcccgagttagaattcgggagctgcttcctttggtgctgatgtttgagctaccaattgctggaattcttcaaccggttcctgatcctaattccccctctattcagcatatatcacaaacgtacaatatttcagtatcatttaaacagcgttcccgaatgtatggtgctactgtcatagtacgagggtctcagaataacactagtgctgtgaaggaaggaactgccatgctgttagaacatcttgctgggagcttagcatcagctattcctgtgagcacacaactagatattgcagctcaacatcatctctttatgatgggtcgaaatgggagcaacatcaaacatatcatgcagagaacaggtgctcagatccactttcctgatcccagtaatccacaaaagaaatctaccgtctacctccagggcaccattgagtctgtctgtcttgcaaggcaatatctcatgggttgtcttcctcttgtgttgatgtttgatatgaaggaagaaattgaagtagatccacaattcattgcgcagttgatggaacagcttgatgtcttcatcagtattaaaccaaagcccaaacagccaagcaagtctgtgattgtgaaaagtgttgagcgaaatgccttaaatatgtatgaagcaaggaaatgtctcctcggacttgaaagcagtggggttaccatagcaaccagtccatccccagcatcctgccctgccggcctggcatgtcccagcctggatatcttagcttcagcaggccttggactcactggactaggtcttttgggacccaccaccttatctctgaacacttcaacaaccccaaactcactcttgaatgctcttaatagctcagtcagtcctttgcaaagtccaagttctggtacacccagccccacattatgggcacccccacttgctaatacttcaagtgccacaggtttttctgctataccacaccttatgattccatctactgcccaagccacattaactaatattttgttgtctggagtgcccacctatgggcacacagctccatctccccctcctggcttgactcctgttgatgtccatatcaacagtatgcagaccgaaggcaaaaaaatctctgctgctttaaatggacatgcacagtctccagatataaaatatggtgcaatatccacttcatcacttggagaaaaagtgctgagtgcaaatcacggggatccgtccatccagacaagtgggtctgagcagacatctcccaaatcaagccccactgaaggttgtaatgatgcttttgttgaagtaggcatgcctcgaagtccttcccattctgggaatgctggtgacttgaaacagatgatgtgtccctccaaggtttcctgtgccaaaaggcagacagtggaactattgcaaggcacgaaaaactcacacttacacagcactgacaggttgctctcagaccctgaactgagtgctaccgaaagccctttggctgacaagaaggctccagggagtgagcgcgctgcagagagggcagcagctgcccagcaaaactccgaaagggcccaccttgctccacggtcatcatatgtcaacatgcaggcatttgactatgaacagaagaagctattagccaccaaagctatgttaaagaaaccagtggtgacggaggtcagaacgcccacaaatacctggagtggcctgggtttttctaaatccatgccagctgaaactatcaaggagttgagaagggccaatcatgtgtcctataagcccacaatgacaaccacttatgagggctcatccatgtccctttcacggtccaacagtcgtgagcacttgggaggtggaagcgaatctgataactggagagaccgaaatggaattggacctggaagtcatagtgaatttgcagcttctattggcagccctaagcgtaaacaaaacaaatcaacggaacactatctcagcagtagcaattacatggactgcatttcctcgctgacaggaagcaatggctgtaacttaaatagctctttcaaaggttctgacctccctgagctcttcagcaaactgggcctgggcaaatacacagatgttttccagcaacaagagatcgatcttcagacattcctcactctcacagatcaggatctgaaggagctgggaataactacttttggtgccaggaggaaaatgctgcttgcaatttcagaactaaataaaaaccgaagaaagctttttgaatcgccaaatgcacgcacctctttcctggaaggtggagcgagtggaaggctaccccgtcagtatcactcagacattgctagtgtcagtggccgctggtag

SEQ ID NO: 37 - DNA, 3213 bp, synthetic construct
  source 1..3213: mol_type = other DNA
atggtcagctggggtcgtttcatctgcctggtcgtggtcaccatggcaaccttgtccctggcccggccctccttcagtttagttgaggataccacattagagccagaagagccaccaaccaaataccaaatctctcaaccagaagtgtacgtggctgcgccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtaacagtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgagatggcagatgatcagggctgtattgaagagcagggggttgaggattcagcaaatgaagattcagtggatgctaagccagaccggtcctcgtttgtaccgtccctcttcagtaagaagaagaaaaatgtcaccatgcgatccatcaagaccacccgggaccgagtgcctacatatcagtacaacatgaattttgaaaagctgggcaaatgcatcataataaacaacaagaactttgataaagtgacaggtatgggcgttcgaaacggaacagacaaagatgccgaggcgctcttcaagtgcttccgaagcctgggttttgacgtgattgtctataatgactgctcttgtgccaagatgcaagatctgcttaaaaaagcttctgaagaggaccatacaaatgccgcctgcttcgcctgcatcctcttaagccatggagaagaaaatgtaatttatgggaaagatggtgtcacaccaataaaggatttgacagcccactttaggggggatagatgcaaaacccttttagagaaacccaaactcttcttcattcaggcttgccgagggaccgagcttgatgatggcatccaggccgactcggggcccatcaatgacacagatgctaatcctcgatacaagatcccagtggaagctgacttcctcttcgcctattccacggttccaggctattactcgtggaggagcccaggaagaggctcctggtttgtgcaagccctctgctccatcctggaggagcacggaaaagacctggaaatcatgcagatcctcaccagggtgaatgacagagttgccaggcactttgagtctcagtctgatgacccacacttccatgagaagaagcagatcccctgtgtggtctccatgctcaccaaggaactctacttcagtcaatag

SEQ ID NO: 38 - DNA, 24 bp, synthetic construct
  source 1..24: mol_type = other DNA
tttttttttttttttttttttttt

(19) United States
(12) Patent Application Publication
(10) Pub. No.: US 2024/0350485 A1
(43) Pub. Date: Oct. 24, 2024

(54) ERDAFITINIB FORMULATIONS AND SYSTEMS FOR INTRAVESICAL ADMINISTRATION

(71) Applicant: TARIS Biomedical LLC, Lexington, MA (US)

(72) Inventors: Srinivas MAMIDI, Raritan, NJ (US); Karen DANIEL, Lexington, MA (US); Dennis GIESING, Lexington, MA (US); Philip Erna H HEYNS, Beerse (BE); Jens Julien Maurits André DHONDT, Beerse (BE); Urbain Alfons C DELAET, Beerse (BE)

(21) Appl. No.: 18/700,089

(22) PCT Filed: Oct. 12, 2022
(86) PCT No.: PCT/US2022/077999

§ 371 Date: Apr. 10, 2024

Related U.S. Application Data
(60) Provisional application No. 63/254,974, filed on Oct. 12, 2021.
(60) Provisional application No. 63/255,387, filed on Oct. 13, 2021.
(60) Provisional application No. 63/311,841, filed on Feb. 18, 2022.

(51) Int. Cl. A61K 31/498; A61J 3/10; A61K 9/00; A61K 9/20; A61M 31/00; A61P 35/00

(52) U.S. Cl. CPC A61K 31/498; A61J 3/10; A61K 9/0034; A61K 9/2009; A61K 9/2013; A61K 9/2018; A61K 9/2027; A61K 9/205; A61K 9/2054; A61K 9/2095; A61M 31/002; A61P 35/00; A61M 2210/1085

(57) ABSTRACT

Provided herein are solid pharmaceutical compositions comprising erdafitinib, processes for making such formulations, and drug delivery systems comprising such formulations, including systems for intravesical administration.

### CROSS-REFERENCE TO RELATED APPLICATIONS

This application claims the benefit of, and priority to, U.S. provisional application 63/254,974 filed Oct. 12, 2021, U.S. provisional application 63/255,387 filed Oct. 13, 2021, and U.S. provisional application 63/311,841 filed Feb. 18, 2022, the contents of each of which is hereby incorporated by reference in its entirety.

### SEQUENCE LISTING

The contents of the electronic sequence listing (761662001940seq.xml; Size: 53,216 bytes; and Date of Creation: Oct. 6, 2022) is herein incorporated by reference in its entirety.

### BACKGROUND

The present disclosure is generally in the field of pharmaceutical formulations and drug-device combination products, and more particularly relates to erdafitinib based formulations and systems for intravesical administration of such formulations.

Erdafitinib (N-(3,5-dimethoxyphenyl)-N′-(1-methylethyl)-N-[3-(1-methyl-1H-pyrazol-4-yl)quinoxalin-6-yl]ethane-1,2-diamine) is a potent pan FGFR kinase inhibitor that binds to and inhibits enzymatic activity of FGFR1, FGFR2, FGFR3 and FGFR4. The synthetic preparation of erdafitinib has been described in WO2011/135376. Erdafitinib has been found to inhibit FGFR phosphorylation and signaling and decrease cell viability in cell lines expressing FGFR genetic alterations, including point mutations, amplifications, and fusions. Erdafitinib has demonstrated antitumor activity in FGFR-expressing cell lines and xenograft models derived from tumor types, including bladder cancer.

Currently, Erdafitinib (BALVERSA®) is available as film-coated tablets for oral administration, and is indicated for the treatment of adult patients with locally advanced or metastatic urothelial carcinoma that has susceptible fibroblast growth factor receptor (FGFR)3 or FGFR2 genetic alterations and progressed during or following at least one line of prior platinum-containing chemotherapy, including within 12 months of neoadjuvant or adjuvant platinum-containing chemotherapy.

U.S. Pat. No. 10,898,482 to Broggini and International Patent Application Publication No. WO 2020/201138 to De Porre describe certain erdafitinib formulations and treatment methods.

Examples of intravesical drug delivery systems are described in U.S. Pat. No. 8,679,094 to Cima et al., U.S. Pat. No. 9,017,312 to Lee et al., U.S. Pat. No. 9,107,816 to Lee et al., and U.S. Pat. No. 9,457,176 to Lee et al. In some embodiments, the intravesical systems include a water permeable housing defining a drug reservoir lumen which contains a solid or semi-solid drug formulation, and release of the drug in vivo occurs by water from the bladder diffusing into drug reservoir lumen to solubilize the drug, and then an osmotic pressure build-up in the drug reservoir lumen drives the solubilized drug out of the drug reservoir lumen through a release aperture.

U.S. Pat. No. 10,286,199 to Lee et al. discloses systems in which drug is released from a housing made of a first wall structure and a hydrophilic second wall structure, wherein the first wall structure is impermeable to the drug and the second wall structure is permeable to the drug. U.S. Pat. No. 10,894,150 to Lee also discloses systems in which drug is released from a housing made of a first wall structure that is impermeable to the drug and a second wall structure that is permeable to the drug.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale.

FIG. 1 is a longitudinal cross-sectional view of one embodiment of a drug delivery system in a coiled retention shape, in accordance with the present disclosure.

FIG. 2 is a traverse cross-sectional view of one embodiment of a drug delivery system, in accordance with the present disclosure.

FIG. 3 is a traverse cross-sectional view of one embodiment of a drug delivery system, in accordance with the present disclosure.

FIG. 4 is a photograph of one embodiment of a drug delivery system loaded with erdafitinib drug tablets, in accordance with the present disclosure.

FIG. 5 is a longitudinal cross-sectional view of one embodiment of a drug delivery system having an elastic retention frame and prior to loading with drug tablets, in a coiled retention shape, in accordance with the present disclosure.

FIG. 6A is a longitudinal cross-sectional view of one embodiment of an elastic retention frame in a coiled retention shape, in accordance with the present disclosure.

FIG. 6B is a partial magnified view of one end of the retention frame of FIG. 6A.

FIG. 7A is a perspective view of one embodiment of a drug delivery system, without drug disposed therein or an elastic retention frame, in a relatively straightened shape, in accordance with the present disclosure.

FIG. 7B is a longitudinal cross-sectional view of the drug delivery system shown in FIG. 7A, taken along line 7B-7B.

FIG. 7C is a traverse cross-sectional view of the drug delivery system shown in FIG. 7A, taken along line 7C-7C.

FIG. 8 is a photograph showing the cross-section of the drug reservoir lumen of a drug delivery system without drug disposed therein, in accordance with the present disclosure.

FIG. 9 shows single-dose erdafitinib exposures in plasma from nude rats bearing subcutaneous or orthotopic UM-UC-1 tumors. Exposure levels were measured in plasma from naïve, orthotopic bladder, or s.c. UM-UC-1 tumor-bearing nude rats. Rats were dosed with a single IVES (1-hour instillation) or p.o. dose of erdafitinib at the dose levels indicated. Individual data points are shown, with the mean represented by a horizontal line at each time point. IVES, intravesical; PO or p.o., oral; s.c., subcutaneous.

FIG. 10 demonstrates the effect of erdafitinib on ERK1/2 phosphorylation in orthotopic bladder UM-UC-1 tumors. Individual pERK and total ERK levels were measured from UM-UC-1 orthotopic bladder tumors from nude rats treated with vehicle, or a single IVES (1-hour instillation) or p.o. dose of erdafitinib at the dose levels indicated. pERK and total ERK levels are reported as a ratio (pERK/ERK) relative to the mean of the vehicle group for the corresponding timepoint, with the exception of the 120-hour timepoint, where values were normalized to the 48-hour vehicle group. Individual data points are shown, with the mean represented by a line at each time point. N=2-6/group; ERK, extracellular signal-regulated kinase; IVES, intravesical; pERK, phosphorylated extracellular signal-regulated kinase; PO or p.o., oral.

FIG. 11 shows the size of orthotopic bladder UC tumor examples versus control bladder at 14 days post implantation. Formalin was used to fix tissue samples after necropsy. UC, urothelial carcinoma; NBTII, rat Nara Bladder Tumor No. 2 cells; T24, human bladder carcinoma cells.

FIG. 12 is a schematic of the perfusion experiment in athymic rats with UM-UC-1 implanted into the bladder wall.

FIG. 13 shows the percentage change in body weight of athymic, bladder-cannulated rats bearing orthotopic UM-UC-1 bladder tumors. Graph values are expressed as mean±SEM of 10-13 animals in each group. Concentrations cited in the figure legend are nominal target urine concentrations. Statistical analysis was carried out by Two-way ANOVA followed by Bonferroni multiple comparison test using Graph Pad Prism (Version 8.3.0). Statistically non-significant difference when percentage change in body weight of erdafitinib (0.5, 1.0, and 5.0 μg/mL) treatment groups were compared with percentage change in body weight of the vehicle control group. SEM, standard error of the mean.

FIG. 14 shows the mean percentage tumor weight reduction after accounting for bladder weight without tumor. Values (Group 1-4) are expressed as mean±SEM of 10-13 animals in each group. Statistical analysis was carried out by One-way ANOVA followed by Dunnett's multiple comparisons test using Graph Pad Prism (Version 8.3.0). Conc, concentration; SEM, standard error of the mean.

FIG. 15 displays the percentage change in body weight of athymic, bladder-cannulated nude rats bearing orthotopic RT-112 bladder tumor. Values are expressed as mean±SEM of 2-14 animals in each group. Concentrations cited in the figure legend are nominal target urine concentrations. Statistical analysis was carried out by Two-way ANOVA followed by Bonferroni multiple comparison test using Graph Pad Prism (Version 8.3.0). Statistically non-significant difference when percentage change in body weight of erdafitinib (0.5, 1.0, and 5.0 μg/mL) treatment groups were compared with percentage change in body weight of vehicle control group except Group 4 on Day 11 (*p<0.05). SEM, standard error of the mean.

FIG. 16 shows the mean bladder weight of athymic nude rats bearing orthotopic RT-112 bladder tumor. Values (Group 1-5) are expressed as mean±SEM of 2-14 animals in each group. Statistical analysis was carried out by One-way ANOVA followed by Dunnett's multiple comparisons test using Graph Pad Prism (Version 8.3.0). * p<0.05. Conc, concentration; SEM, standard error of the mean; ns, not significant.

FIGS. 17A and 17B show the plasma (FIG. 17A) and bladder (FIG. 17B) concentrations in rats following bladder perfusion of erdafitinib. Bladder perfusion of erdafitinib solution (0.1 mg/mL, 0.1 mL/hour, cumulative dose 0.72 mg) took place over 72 hours. Concentrations are expressed as average daily urine concentration in ng/mL.

FIG. 18 shows mean erdafitinib urine concentrations in pigs following bladder perfusion of erdafitinib for 7 days. Conc., concentration; SD, standard deviation.

FIG. 19 shows mean erdafitinib plasma concentrations in pigs following bladder perfusion of erdafitinib for 7 days. SD, standard deviation.

FIG. 20 shows the screening results of materials permeation. O, permeable; Δ, practically impermeable; X, impermeable. ^{a} High variability between replicates.

FIG. 21 shows the predicted (from short core) and actual (from full-length) average release rate profiles for permeation prototypes. Erda, erdafitinib releasing intravesical system; HPbCD, hydroxypropyl β-cyclodextrin.

FIG. 22 displays the average release rate profile for erdafitinib free base+HP-β-CD permeation prototype (EG-80A stripe material). Erda, erdafitinib; HP-β-CD, hydroxypropyl β-cyclodextrin; SU, simulated urine.

FIG. 23 shows the average release rate profile for erdafitinib free base permeation prototypes, with and without HP-β-CD (HP-60D-35 stripe material). Erda, erdafitinib; HP-β-CD or HPbCD, hydroxypropyl β-cyclodextrin; SU, simulated urine.

FIG. 24 shows the IVR (in vitro release) profile for Prototype 1 (permeation, erdafitinib free base, tablets, wireform). Erda, erdafitinib; IVR, in vitro release.

FIG. 25 shows the IVR profile for Prototype 2 (permeation, erdafitinib free base+HP-β-CD (10% w/w), tablets, wireform). Erda, erdafitinib; HP-β-CD, hydroxypropyl β-cyclodextrin; IVR, in vitro release.

FIG. 26 summarizes the in vivo release rate versus time profiles in minipigs for Prototypes 1 and 2.

FIG. 27 summarizes the mean urine concentration versus time profiles in minipigs for Prototypes 1 and 2.

FIG. 28A is a diagram of an exemplary permeation system where the base material is impermeable TPU and the stripe material is permeable TPU. FIG. 28B shows an overview of an exemplary permeation design. TPU or tPU, thermoplastic polyurethane; API, active pharmaceutical ingredient; HP-β-CD, hydroxypropyl β-cyclodextrin.

FIG. 29 provides an overview of the solubility of the erdafitinib free base drug as a function of pH at 20° C. ^{a} USP/Ph. Eur. Terminology.

FIG. 30A demonstrates the solubility of the erdafitinib free base drug as a function of pH at 37° C. using HCl to adjust the pH. Expon., exponential.

FIGS. 31A-31B shows the solubility of the erdafitinib free base drug and erdafitinib HCl salt Form 1 as a function of pH in simulated urine at 37° C. (FIG. 31A), as well as the solubility of the erdafitinib free base drug as a function of pH in simulated urine at 37° C. in mg/mL (FIG. 31B). HP-β-CD, hydroxypropyl β-cyclodextrin; Sim urine, simulated urine.

### DETAILED DESCRIPTION

In some embodiments, erdafitinib solid formulations are provided containing a high concentration of erdafitinib, which are designed for intravesical drug delivery and controlled and extended drug release when deployed within the bladder. In some embodiments, the solid erdafitinib formulations are further tailored for large scale manufacturing and to provide structural and chemical integrity of the solid formulations, in particular tablets, when used in an intravesical drug delivery system. Improved intravesical drug delivery systems, methods of manufacturing the same, and methods of drug delivery are also provided. In a particular embodiment, systems are configured for intravesical insertion and sustained drug delivery, preferably providing a zero order release rate of therapeutically effective amounts of the drug, in particular erdafitinib.

Described herein the development of erdafitinib formulations and release systems that are tailored for intravesical drug delivery, in order to take advantage of this route of administration. When formulated in solid form and administered in a suitable intravesical drug delivery system, such formulations might provide a controlled drug release rate and an extended drug release profile. Further provided are systems capable of delivering erdafitinib at effective release rates for the local treatment of bladder cancer.

Erdafitinib exhibits pH-dependent solubility over the normal urine pH range of 5.5 to 7. In some embodiments, the formulations and release systems are tailored to minimize the effect of urine pH and composition on system release rate.

In particular embodiments, the drug delivery system described herein is a drug device combination, consisting of a device constituent, particularly an intravesical device, and a drug constituent, particularly an erdafitinib formulation, such as erdafitinib tablets.

### Certain Terminology

Recurrence-Free Survival (RFS) is defined as the time from randomization to the first detection of high-grade Ta or T1 bladder cancer or positive urine cytology.

Complete Response (CR) is defined as the absence of urothelial carcinoma by cystoscopy, confirmed pathologically at first assessment, and negative urine cytology.

Duration of CR is defined as the time from first documentation of CR until the date of documented recurrence or progression, or death, whichever comes first.

Pathological Complete Response (pCR) Rate is defined as percentage of participants with no pathologic evidence of intravesical disease (pT0) and no pathologic evidence of nodal involvement (pN0).

No Pathologic Evidence of Intravesical Disease (pT0) rate is defined as percentage of participants with no Pathologic Evidence of Intravesical Disease.

Rate of downstaging to Less than (<) pT2 s defined as percentage of participants with pT stage<2.

When used herein wt % in relation to drug or excipient(s) refers to weight % based on the total weight of the formulation concerned, unless otherwise indicated.

### The Erdafitinib Formulation & Tablets

In one aspect, this disclosure provides erdafitinib formulations, in particular erdafitinib tablets suitable for use in the disclosed intravesical drug delivery system. In particular, drug tablets comprising erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) are provided. As another example, drug tablets comprising erdafitinib HCl salt are provided. After the drug delivery system is inserted intravesically, the drug is released from the system into the bladder. In an aspect for example, the drug delivery system may operate by diffusion, which produces a continuous release of the drug into the bladder over an extended period as the drug is released from the tablets in the system.

In order to increase or maximize the amount of drug that can be stored in and released from the disclosed drug delivery system, the drug tablets can have a relatively high erdafitinib content by weight. This relatively high weight fraction of erdafitinib in the drug tablet is attended by a reduced or low weight fraction of excipients which may be required for tablet manufacturing and system assembly and drug use considerations. For the purposes of this disclosure, terms such as “weight fraction,” “weight percentage,” and “percentage by weight” with reference to any drug or API (active pharmaceutical ingredient) refers to the drug or API in the form employed, whether in free base form, free acid form, salt form, or hydrate form. For example, a drug tablet that has 90% by weight (90 wt %) of a drug or excipient in salt form may include less than 90% by weight of that drug in free base form. Unless otherwise specified, weight percentages are relative to the entire solid pharmaceutical composition.

The erdafitinib drug tablet of this disclosure includes an erdafitinib content and an excipient content. The drug content can include one form or more than one form of erdafitinib, such as free base or salt form, and the excipient content can include one or more excipients. Particular embodiments include erdafitinib free base API, and the example formulations presented herein comprise the erdafitinib free base API. The term “excipient” is known in the art, and representative examples of excipients useful in the disclosed drug tablets may include but are not limited to ingredients such as binders, lubricants, glidants, disintegrants, solubilizers, colorants, fillers or diluents, wetting agents, stabilizers, formaldehyde scavengers, coatings, and preservatives, or any combination thereof, as well as other ingredients to facilitate manufacturing, storing, or administering the drug tablet.

Another aspect of this disclosure provides a process for making a solid pharmaceutical composition, in which the process can comprise: (a) preparing an intragranular solid composition comprising or consisting essentially of (i) erdafitinib free base and (ii) at least one intragranular pharmaceutical excipient; (b) combining the intragranular solid composition with at least one extragranular pharmaceutical excipient to form a blend; and (c) tableting the blend to form the solid pharmaceutical composition. In embodiments, the erdafitinib free base can be present in a concentration of at least 45 wt % of the solid pharmaceutical composition. The at least one intragranular pharmaceutical excipient and at least one extragranular pharmaceutical excipient can comprise or can be selected from at least one common (mutually occurring) pharmaceutical excipient, or there can be no common (mutually occurring) pharmaceutical excipient between the intragranular excipients and the extragranular pharmaceutical excipients. The solid pharmaceutical composition can be made by a process that includes an intragranular solid composition prepared by a roller compaction process or by a fluid bed granulation process. In some embodiments, the step of (a) preparing an intragranular solid composition comprises: (1) preparing a pre-blend comprising the erdafitinib free base and one or more excipients; (2) preparing a binder solution; and (3) preparing the intragranular solid composition by combining the pre-blend and the binder solution. In some embodiments, the step of (a) preparing an intragranular solid composition comprises: (1) preparing a pre-blend comprising the erdafitinib free base and one or more excipients; (2) preparing a binder solution; and (3) preparing the intragranular solid composition by combining the pre-blend and the binder solution by a fluid bed granulation process. In some embodiments, the step of (a) preparing an intragranular solid composition comprises: (1) preparing a pre-blend comprising the erdafitinib free base with a stabilizer, a solubilizer, and a filler; (2) preparing a binder solution comprising a binder and a solvent; and (3) preparing the intragranular solid composition by combining the pre-blend and the binder solution by a fluid bed granulation process. In some embodiments, the step of (a) preparing an intragranular solid composition comprises: (1) preparing a pre-blend comprising the erdafitinib free base, meglumine, hydroxypropyl-beta-cyclodextrin, and microcrystalline cellulose; (2) preparing a binder solution comprising hydroxypropyl methylcellulose and purified water; and (3) preparing the intragranular solid composition by combining the pre-blend and the binder solution by a fluid bed granulation process. In some embodiments, the step of (a) preparing an intragranular solid composition comprises: (1) preparing a pre-blend comprising the erdafitinib free base with a solubilizer and a filler; (2) preparing a binder solution comprising a binder and a solvent; and (3) preparing the intragranular solid composition by combining the pre-blend and the binder solution by a fluid bed granulation process. In some embodiments, the step of (a) preparing an intragranular solid composition comprises: (1) preparing a pre-blend of the erdafitinib free base, hydroxypropyl-beta-cyclodextrin, and microcrystalline cellulose; (2) preparing a binder solution comprising hydroxypropyl methylcellulose and purified water; and (3) preparing the intragranular solid composition by combining the pre-blend and the binder solution by a fluid bed granulation process.

Another aspect of this disclosure provides a process for making a solid pharmaceutical composition, in which the process can comprise: (a) preparing an intragranular solid composition comprising or consisting essentially of (i) erdafitinib HCl salt form and (ii) at least one intragranular pharmaceutical excipient; (b) combining the intragranular solid composition with at least one extragranular pharmaceutical excipient to form a blend; and (c) tableting the blend to form the solid pharmaceutical composition. In embodiments, the erdafitinib HCl salt form can be present in a concentration of at least 45 wt % of the solid pharmaceutical composition. The at least one intragranular pharmaceutical excipient and at least one extragranular pharmaceutical excipient can comprise or can be selected from at least one common (mutually occurring) pharmaceutical excipient, or there can be no common (mutually occurring) pharmaceutical excipient between the intragranular excipients and the extragranular pharmaceutical excipients. The solid pharmaceutical composition can be made by a process that includes an intragranular solid composition prepared by a roller compaction process or by a fluid bed granulation process.

In embodiments, the erdafitinib drug tablets includes erdafitinib in its free base form. Other embodiments of the erdafitinib drug tablets can include erdafitinib in a salt form. In one aspect, erdafitinib drug tablets can include greater than or equal to 40 wt % erdafitinib free base, with the remainder of the weight comprising excipients, such as lubricants, binders, and stabilizers that facilitate making and using the drug tablet. Alternatively, the erdafitinib drug tablets can include greater than or equal to 45 wt %, greater than or equal to 50 wt %, greater than or equal to 55 wt %, or greater than or equal to 60 wt % erdafitinib free base. In each of these weight percentage embodiments, the practical upper limit of erdafitinib free base in the tablet formulation is about 65 wt %, or 70 wt %. Therefore, in an aspect, the drug tablets can include from 40 wt % to 60 wt % of erdafitinib in its free base form, or from 45 wt % to 55 wt % of erdafitinib in its free base form. In some embodiments of the foregoing, the drug tablets can include between about 5% and about 15% by weight of hydroxypropyl-β-cyclodextrin (HP-3-CD). In some embodiments of the foregoing, the drug tablets can include about 10% by weight of hydroxypropyl-β-cyclodextrin (HP-3-CD). In embodiments, the drug tablets can include 50% by weight of erdafitinib in its free base form, based on the total weight of the tablet. In embodiments, the drug tablets can include 50% by weight of erdafitinib in its free base form, and between about 5% and about 15% by weight of hydroxypropyl-β-cyclodextrin (HP-3-CD) based on the total weight of the tablet. In embodiments, the drug tablets can include 50% by weight of erdafitinib in its free base form, and 10% by weight of hydroxypropyl-β-cyclodextrin (HP-3-CD) based on the total weight of the tablet.

In embodiments, the erdafitinib drug tablets includes erdafitinib in its HCl salt form. In one aspect, erdafitinib drug tablets can include greater than or equal to 40 wt % erdafitinib HCl salt form, with the remainder of the weight comprising excipients, such as lubricants, binders, and stabilizers that facilitate making and using the drug tablet. Alternatively, the erdafitinib drug tablets can include greater than or equal to 45 wt %, greater than or equal to 50 wt %, greater than or equal to 55 wt %, or greater than or equal to 60 wt % erdafitinib HCl salt form. In each of these weight percentage embodiments, the practical upper limit of erdafitinib salt form in the tablet formulation is about 65 wt %, or 70 wt %. Therefore, in an aspect, the drug tablets can include from 40 wt % to 60 wt % of erdafitinib in its HCl salt form, or from 45 wt % to 55 wt % of erdafitinib in its HCl salt form. In embodiments, the drug tablets can include 50% by weight of erdafitinib in its HCl salt form, based on the total weight of the tablet.

In one embodiment, the erdafitinib drug and excipients are selected and the tablet is formulated to permit release of the drug from the tablet. In some embodiments, the erdafitinib drug and excipients are selected and the tablet is formulated to permit solubilization of the drug from the tablet. In embodiments, the erdafitinib is formulated in a pharmaceutical composition to be sterilizable, either within or outside of the drug delivery system, without resulting in substantial or detrimental changes to the chemical or physical composition of the drug tablets which would otherwise make them unsuitable for delivering the erdafitinib as described herein. In an aspect, the erdafitinib drug and excipients are selected for their suitability for sterilization processes. In an embodiment, the drug delivery system comprising the drug tablets is sterilized as a whole. In particular, the drug delivery system comprising the drug tablets is sterilized by gamma irradiation.

In an aspect, the erdafitinib drug tablets may be sized and shaped for use with an implantable drug delivery system including the intravesical drug delivery system disclosed herein. For example, the erdafitinib drug tablets may be “mini-tablets” that are generally smaller in size than conventional tablets, which may permit inserting the system-housed drug tablets through a lumen such as the urethra into a cavity such as the bladder. The erdafitinib tablets may be coated or uncoated. In particular, uncoated tablets formulated according to this disclosure have been found to work well in combination with the system.

In embodiments, the drug tablet for intravesical insertion or other in vivo implantation can be in the form of a solid cylinder having a cylindrical axis, a cylindrical side face, circular end faces perpendicular to the cylindrical axis, a diameter across the circular end faces, and a length along the cylindrical side face. In cylindrical form, each mini-tablet can have a length (L) exceeding its diameter (D) so that the mini-tablet has an aspect ratio (L:D) of greater than 1:1. For example, the aspect ratio (L:D) of each mini-tablet can be 1.6, 1.5, 1.4, 1.3, 1.2, 1.1, or range in values between these aspect ratios. Embodiments of the mini-tablet can have a cylindrical diameter of from 1.0 mm to 3.2 mm, or from 1.5 mm to 3.1 mm, or from 2.0 mm to 2.7 mm, or from 2.5 mm to 2.7 mm. In some aspects, the mini-tablet can have a length of from 1.7 mm to 4.8 mm, or from 2.0 mm to 4.5 mm, or from 2.8 mm to 4 mm, or from 3 mm to 3.5 mm.

The API used in the solid tablet formulations can be erdafitinib, which is N-(3,5-dimethoxyphenyl)-N′-(1-methylethyl)-N-[3-(1-methyl-1H-pyrazol-4-yl)quinoxalin-6-yl]ethane-1,2-diamine, and the chemical structure of which is illustrated below. Erdafitinib tablets for use in the disclosed intravesical system can be formulated using the erdafitinib free base or a salt thereof. In an aspect, the erdafitinib tablets for use in the disclosed intravesical system can include erdafitinib free base. In an aspect, the erdafitinib tablets for use in the disclosed intravesical system can include erdafitinib HCl salt, particularly erdafitinib HCl salt which is in a crystalline form. In some embodiments of the foregoing, the erdafitinib tablets for use in the disclosed intravesical system can include erdafitinib free base which is in a crystalline form. As described herein, including certain stabilizers, solubilizers, and excipients in the erdafitinib free base formulation can provide advantageous stabilizing and dissolution properties for effective use of the free base formulation in the disclosed intravesical system.

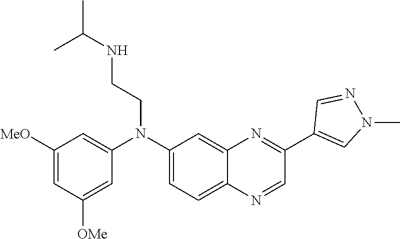 [SMILES: COc1cc(OC)cc(N(CCNC(C)C)c2ccc3ncc(-c4cnn(C)c4)nc3c2)c1]

In embodiments, the erdafitinib drug tablet can incorporate various excipients which include, but are not limited to, at least one solubilizer, at least one binder, at least one wetting agent, at least one disintegrant, at least one stabilizer, at least one diluent, at least one glidant, at least one lubricant, and the like, or any combination thereof. Any excipient or any combination of the excipients can be present in the intragranular solid composition, the extragranular solid composition, or both the intragranular and the extragranular solid composition. In an aspect, at least one intragranular pharmaceutical excipient and at least one extragranular pharmaceutical excipient can be the same, that is, can be selected from at least one common (mutually occurring) pharmaceutical excipient. In a further aspect, the intragranular pharmaceutical excipients and the extragranular pharmaceutical excipients do not comprise a common (mutually occurring) pharmaceutical excipient, such that the intragranular and the extragranular excipients are mutually exclusive. In embodiments, the erdafitinib drug tablet, in particular the erdafitinib drug tablet comprising from 40 wt % to 70 wt %, or from 40 wt % to 60 wt %, or from 45 wt % to 55 wt %, for example, 50 wt % of erdafitinib, includes at least one solubilizer, at least one binder, at least one stabilizer, at least one diluent, at least one glidant, at least one lubricant, and the like, or any combination thereof. In embodiments, the erdafitinib drug tablet, in particular the erdafitinib drug tablet comprising from 40 wt % to 70 wt %, or from 40 wt % to 60 wt %, or from 45 wt % to 55 wt %, for example, 50 wt % of erdafitinib, includes at least one solubilizer, at least one binder, at least one diluent, at least one glidant, at least one lubricant, and the like, or any combination thereof.

It will be appreciated that these functional descriptions of various excipients are used generally as follows. A solubilizer can improve or enhance the solubility of the API such as erdafitinib free base within the drug lumen of the disclosed system or within a body cavity such as the bladder once the API is released from the system. A binder can hold the solid particles of the composition together for physical stability. A wetting agent can lower the surface tension between the drug and the medium in which it occurs and help maintain the solubility of the drug. A disintegrant can aid in the minitablet disintegration when contacting water to release the drug substance. A stabilizer can improve the chemical stability such as the thermal stability of the formulation, including the API, or protects the API against degradation. A diluent can function as a bulking agent to increase the volume or weight of the composition which may aid in providing tablet of the desired size or which may aid in tabletability of the API-excipient blend. A glidant may improve the flow properties of the (granulated) particles of tablet components or of the powder blend to be tableted. A lubricant can prevent particles of the composition from adhering to components of the manufacturing apparatus, such as dies and punches of a tablet press. In an aspect, an excipient can be water soluble. In another aspect, an excipient can be colloidal in water. According to another aspect, an excipient can be soluble under the conditions of its deployment in the patient, such as in a bladder. These and other excipients are described in more detail below.

Stabilizers such as Formaldehyde Scavengers

In an aspect, erdafitinib API may be sensitive to degradation under certain conditions when incorporated into a solid formulation. For example, erdafitinib can degrade or transform in the presence of formaldehyde, to form the cyclization product 6,8-dimethoxy-4-(1-methylethyl)-1-[3-(1-methyl-1H-pyrazol-4-yl)quinoxalin-6-yl]-2,3,4,5-tetrahydro-1H-1,4-benzodiazepine. Formaldehyde can come into contact with the erdafitinib from a variety of sources in the environment, such as from packaging materials or as a contaminant in excipients or other components of the formulation.

Accordingly, in one aspect, the erdafitinib pharmaceutical formulation can include a formaldehyde scavenger to improve the stability or shelf life of the formulation. Various formaldehyde scavengers can be employed which can prevent, slow down, diminish, or postpone the formation of degradation products when erdafitinib contacts formaldehyde. Therefore, the erdafitinib pharmaceutical formulation stability such as its chemical stability can be increased in the presence of a formaldehyde scavenger as compared to a erdafitinib pharmaceutical formulations absent a formaldehyde scavenger. In an aspect, the formaldehyde scavenger can be present in the solid pharmaceutical composition as a component of the intragranular solid composition, the extragranular solid composition, or both the intragranular and extragranular solid composition. In an aspect, the formaldehyde scavenger, in particular meglumine, is present in the solid pharmaceutical composition as a component of the intragranular solid composition.

Formaldehyde scavengers can include or can be selected from compounds comprising a reactive nitrogen center, such as compounds containing amine or amide groups. Without being bound by theory, it is thought that these compounds can react with formaldehyde to form a Schiff base imine (R¹R²C═NR³, where R³ is not hydrogen), which itself can bind formaldehyde. Examples of such formaldehyde scavengers include but are not limited to amino acids, amino sugars, alpha-(α-)amine compounds, conjugates and derivatives thereof, and mixtures thereof. Such formaldehyde scavenger compounds can include two or more amine and/or amide moieties which can scavenge formaldehyde.

In an aspect, formaldehyde scavengers can include or can be selected from, for example, meglumine, glycine, alanine, serine, threonine, cysteine, valine, leucine, isoleucine, methionine, phenylalanine, tyrosine, aspartic acid, glutamic acid, arginine, lysine, ornithine, taurine, histidine, aspartame, proline, tryptophan, citrulline, pyrrolysine, asparagine, glutamine, tris(hydroxymethyl)aminomethane, conjugates thereof, pharmaceutically acceptable salts thereof, or any combination thereof. According to an aspect, the formaldehyde scavenger can include or can be selected from meglumine or a pharmaceutically acceptable salt thereof, in particular meglumine base.

Therefore, an aspect of this disclosure is the use of a formaldehyde scavenger, in particular meglumine, in an erdafitinib pharmaceutical formulation such as a drug tablet formulation, to increase the stability of erdafitinib in any of its forms, including erdafitinib free base, a salt thereof, or a solvate thereof. The chemical stability of the erdafitinib pharmaceutical formulation is increased as compared to an erdafitinib pharmaceutical formulation or composition containing no formaldehyde scavenger. An aspect of the disclosure is a method of preventing, slowing down, diminishing, or postponing the formation of degradation products such as the following compound, which can form from erdafitinib in the presence of formaldehyde:

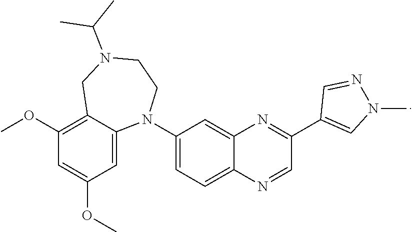

In an aspect, degradation products such as the above can occur in a solid tablet composition such as a mini-tablet formulation, in particular in a mini-tablet as disclosed herein.

When present in the erdafitinib solid pharmaceutical composition, the formaldehyde scavenger can be present in the solid pharmaceutical composition in a concentration of from 0.01 wt % to 5 wt %, from 0.05 wt % to 3 wt %, from 0.1 wt % to 2 wt %, from 0.5 wt % to 1.5 wt %, or about 1 wt %. In some embodiments, when present in the erdafitinib solid pharmaceutical composition, the formaldehyde scavenger can be present at a concentration of about 1 wt %. When present in the erdafitinib solid pharmaceutical composition, the formaldehyde scavenger can be present in the solid pharmaceutical composition in a concentration of, for example, from 5 wt % to 10 wt %, about 5 wt %, about 6 wt %, about 7 wt %, about 8 wt %, about 9 wt % or about 10 wt %. In some embodiments, the erdafitinib solid pharmaceutical composition contains erdafitinib free base, and the formaldehyde scavenger is present. In some embodiments, the erdafitinib solid pharmaceutical composition contains erdafitinib free base, and the formaldehyde scavenger is present in the solid pharmaceutical composition in a concentration of from 0.01 wt % to 5 wt %, from 0.05 wt % to 3 wt %, from 0.1 wt % to 2 wt %, from 0.5 wt % to 1.5 wt %, or about 1 wt %. In some embodiments, the erdafitinib solid pharmaceutical composition contains erdafitinib free base, and the formaldehyde scavenger is present in the solid pharmaceutical composition in a concentration of about 1 wt %. In some embodiments of any of the foregoing, the formaldehyde scavenger is meglumine.

In some embodiments, the pharmaceutical compositions as described herein, in particular the erdafitinib drug tablets, do not contain a stabilizer or formaldehyde scavenger.

### Solubilizers

In an aspect, the erdafitinib formulation can include a solubilizer. The solubilizer can be in the intragranular component, the extragranular component, or both the intragranular and extragranular component of the formulation. In embodiments, the solubilizer can comprise or can be selected from, for example (a) a cyclic oligosaccharide, (b) a cellulose which is functionalized with methoxy-, 2-hydroxypropoxy-, acetyl-, or succinoyl-moieties or a combination thereof, or (c) a salt thereof. In an embodiment, the solubilizer is present in the intragranular component.

In embodiments, solubilizers for the erdafitinib tablet formulation can comprise or can be selected from an oligosaccharide. In embodiments, the solubilizer can comprise or can be selected from a cyclic oligosaccharide such as a cyclodextrin. Suitable cyclodextrin solubilizers for the erdafitinib tablet formulation include, but are not limited to, hydroxypropyl-beta-cyclodextrin, hydroxypropyl-gamma-cyclodextrin, sulfobutyl ether-beta-cyclodextrin sodium salt, or any combination thereof. In other embodiments, the solubilizer can comprise or can be hydroxypropyl methylcellulose acetate succinate, hydroxypropyl methylcellulose E5 (HPMC-E5), or a combination thereof.

Oligosaccharide solubilizers can be present in erdafitinib tablet formulation, for example a erdafitinib free base formulation, in a concentration of from 1 wt % to 20 wt %, alternatively from 3 wt % to 18 wt %, alternatively from 5 wt % to 15 wt %, alternatively from 7 wt % to 12 wt %, or alternatively 10 wt % or about 10 wt %. The cyclodextrin solubilizer can be present in an erdafitinib tablet formulation, for example an erdafitinib free base formulation, in a concentration of 1 wt %, 2 wt %, 3 wt %, 4 wt %, 5 wt %, 6 wt %, 7 wt %, 8 wt % 9 wt % 10 wt %, 11 wt %, 12 wt %, 13 wt %, 14 wt %, 15 wt %, 16 wt %, 17 wt %, 18 wt %, 19 wt %, or 20 wt %, or any range between any of these weight percentages.

In an aspect, a solubilizer for the erdafitinib tablet formulation disclosed herein can comprise or can be hydroxypropyl-beta-cyclodextrin (HP-β-CD). One embodiment of an erdafitinib free base formulation includes a hydroxypropyl-beta-cyclodextrin solubilizer, in particular an erdafitinib free base formulation including hydroxypropyl-beta-cyclodextrin in from 8 wt % to 12 wt %, or alternatively, 10 wt % or about 10 wt % concentration. In some embodiments, the formulation comprises hydroxypropyl-beta-cyclodextrin at about 10 wt % concentration. In this formulation, the erdafitinib free base API can be present in a concentration of from 40 wt % to 70 wt %, or from 40 wt % to 60 wt %, or from 45 wt % to 55 wt %, for example, 50 wt %. In an embodiment, the hydroxypropyl-beta-cyclodextrin is present in the intragranular solid composition. In embodiments, the drug tablets can include 50% by weight of erdafitinib in its free base form, 1% by weight of meglumine, and hydroxypropyl-beta-cyclodextrin in from 8 wt % to 12 wt %, or alternatively, 10 wt % or about 10 wt % concentration. In embodiments, the drug tablets can include 50% by weight of erdafitinib in its free base form, 10% by weight of hydroxypropyl-β-cyclodextrin (HP-3-CD), and 1% by weight of meglumine based on the total weight of the tablet. In embodiments, the drug tablets can include at least about 45% by weight of erdafitinib in its free base form, 10% by weight of hydroxypropyl-β-cyclodextrin (HP-β-CD), and 0% by weight of meglumine based on the total weight of the tablet. In embodiments, the drug tablets can include 50% by weight of erdafitinib in its free base form, 10% by weight of hydroxypropyl-β-cyclodextrin (HP-β-CD), and 0% by weight of meglumine based on the total weight of the tablet.

### Binders

Pharmaceutical excipients for the erdafitinib solid pharmaceutical composition may include one or more binders. The one or more binders can be present in the solid pharmaceutical composition as a component of the intragranular solid composition, the extragranular solid composition, or both the intragranular and extragranular solid composition. Suitable binders can be water soluble, water insoluble, or slightly water soluble or combinations of these. In an aspect, binders can include polymeric binders such as water soluble polymeric binders, slightly water soluble polymeric binders, water insoluble polymeric binders, or any combination thereof. Polymeric binders can include non-ionic polymers.

It will be appreciated by the person of ordinary skill that binders may also function as a diluent (also termed filler) in a pharmaceutical composition. Accordingly, binders provided in this disclosure may also be used for their diluent function as appropriate and unless otherwise indicated.

In an aspect, suitable binders can include or can be selected from polyvinylpyrrolidone (PVP, also termed polyvidone, povidone, or poly(1-vinyl-2-pyrrolidinone)), poly(vinyl acetate) (PVA), vinylpyrrolidone-vinyl acetate copolymer, polyethylene oxide (PEO, also termed poly(ethylene glycol) or PEG), polypropylene oxide (PPO, also termed poly(propylene glycol) or PPG), an ethylene glycol-propylene glycol copolymer, a poloxamer, hydroxypropyl cellulose (HPC), hydroxypropyl methylcellulose (HPMC), microcrystalline cellulose, silicified microcrystalline cellulose, or combinations thereof. In an aspect, suitable binders can include or can be selected from polyvinylpyrrolidone (PVP, also termed polyvidone, povidone, or poly(1-vinyl-2-pyrrolidinone)), poly(vinyl acetate) (PVA), vinylpyrrolidone-vinyl acetate copolymer, polyethylene oxide (PEO, also termed poly(ethylene glycol) or PEG), polypropylene oxide (PPO, also termed poly(propylene glycol) or PPG), an ethylene glycol-propylene glycol copolymer, a poloxamer, hydroxypropyl cellulose (HPC), hydroxypropyl methylcellulose (HPMC), microcrystalline cellulose, or combinations thereof. In an aspect, suitable binders can include or can be selected from hydroxypropyl methylcellulose (HPMC), microcrystalline cellulose, vinylpyrrolidone-vinyl acetate copolymer, or combinations thereof. In an aspect, suitable binders can include or can be selected from hydroxypropyl methylcellulose (HPMC), vinylpyrrolidone-vinyl acetate copolymer (copovidone), or combinations thereof. In some embodiments, the binder may be hydroxypropyl methylcellulose (HPMC). In some embodiments, the binder may be hydroxypropyl methylcellulose (HPMC) at a concentration of about 1.5 wt % of the solid composition. In some embodiments, the binder may be hydroxypropyl methylcellulose (HPMC) at 1.5 wt % of the solid composition, and is present in the intragranular solid composition.

In further aspects, suitable binders can include or can be selected from polymers of or copolymers of vinylpyrrolidone (VP, also 1-vinyl-2-pyrrolidinone) and vinyl acetate (VA). Such copolymers of VP and VA may also be referred to as “copovidones”. Suitable binders also may include or may be selected from polymers of or copolymers of ethylene oxide (EO) and propylene oxide (PO). Again, these binders can be used in combinations with other binders such as in combination with microcrystalline cellulose, hydroxypropyl cellulose (HPC), or hydroxypropyl methylcellulose (HPMC).

In an aspect, the total concentration of the at least one binder in the solid pharmaceutical composition can be from 1 wt % to 30 wt %, from 2 wt % to 30 wt %, from 5 wt % to 30 wt %, from 5 wt % to 25 wt %, from 10 wt % to 25 wt %, from 10 wt % to 22 wt %, from 12 wt % to 22 wt %, from 14 wt % to 19 wt %, or from 12 wt % to 19 wt %.

According to another aspect, suitable polymeric binders can include or can be selected from a copolymer of vinylpyrrolidone and vinyl acetate, which can be termed poly(vinylpyrrolidone-co-vinyl acetate) or poly(VP-co-VA). Examples of suitable poly(vinylpyrrolidone-co-vinyl acetate) binders include Kollidon® VA64 and Kollidon® VA64 Fine (BASF, Ludwigshafen am Rhein, Germany), having a molecular weight (Mw) range of from 45,000 g/mol to 70,000 g/mol based on measuring the light scatter of a solution. Another suitable binder is Kollidon® K30.

In embodiments, the polymeric binders such as the vinylpyrrolidone-vinyl acetate copolymer can be present in the disclosed erdafitinib tablet formulation in a concentration of from 2 wt % to 15 wt %, alternatively from 4 wt % to 12 wt %, alternatively from 6 wt % to 10 wt %, or alternatively, 8 wt % or about 8 wt %. For example, the vinylpyrrolidone-vinyl acetate copolymer binder can be present in erdafitinib tablet formulation, for example a erdafitinib free base formulation, in a concentration of 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt % or any range between any of these weight percentages e.g., 7.5 wt %. In an aspect, the vinylpyrrolidone-vinyl acetate copolymer is present at a concentration of 8 wt % of the solid composition. In an aspect, the vinylpyrrolidone-vinyl acetate copolymer is present in the intragranular solid composition. In an aspect, the vinylpyrrolidone-vinyl acetate copolymer is present in the intragranular solid composition and said intragranular solid composition is prepared by roller compaction. In an aspect, the vinylpyrrolidone-vinyl acetate copolymer is present in the intragranular solid composition and said intragranular solid composition is prepared by fluid bed granulation. In an aspect, the vinylpyrrolidone-vinyl acetate copolymer is present in the extragranular solid composition. In an aspect, the vinylpyrrolidone-vinyl acetate copolymer is present at a concentration of about 7.5 wt % of the solid composition. In an aspect, the vinyl-pyrrolidone-vinyl acetate copolymer is present at a concentration of about 7.5 wt % of the solid composition, and is in the extragranular solid composition.

In an aspect, the binder can comprise or can be microcrystalline cellulose. For example, the microcrystalline cellulose can be present in the solid pharmaceutical composition in a concentration of from 5 wt % to 30 wt %, from 10 wt % to 20 wt %, from 5 wt % to 20 wt %, from 6 wt % to 15 wt %, or from 7 wt % to 12 wt %. For example, the microcrystalline cellulose can be present in the solid pharmaceutical composition as a filler and/or as a binder at a concentration of about 17.5 wt %. For example, the microcrystalline cellulose can be present in the solid pharmaceutical composition at a concentration of about 17.5% wt % of the solid composition and is present in the intragranular solid composition and extragranular solid composition. For example, the microcrystalline cellulose can be present in the solid pharmaceutical composition as a filler in the intragranular composition, at a concentration of about 10 wt % of the solid composition, and can be present in the solid pharmaceutical composition as a binder in the extragranular composition, at a concentration of about 7.5 wt % of the solid composition.

According to another aspect, the binder can comprise or can be silicified microcrystalline cellulose. For example, the silicified microcrystalline cellulose can be present in the solid pharmaceutical composition in a concentration of from 3 wt % to 18 wt %, from 4 wt % to 15 wt %, or from 5 wt % to 12 wt %.

In a further aspect, the binder can comprise or can be hydroxypropyl methylcellulose (HPMC). For example, the hydroxypropyl methylcellulose (HPMC) can be present in the solid pharmaceutical composition in a concentration of from 0.25 wt % to 5 wt %, from 0.5 wt % to 4 wt %, or from 0.75 wt % to 3 wt %. In an aspect, the IPMC binder can be present in the solid pharmaceutical composition in the intragranular solid composition.

### Wetting Agents

Pharmaceutical excipients for the erdafitinib solid pharmaceutical composition may include one or more wetting agents. The one or more wetting agents can be present in the solid pharmaceutical composition in the intragranular solid composition, the extragranular solid composition, or both the intragranular and extragranular solid compositions. In exemplary embodiments, the wetting agent can comprise or can be selected independently from an anionic surfactant or a non-ionic surfactant, in particular an anionic surfactant. For example, the wetting agent can comprise or can be selected independently from sodium lauryl sulfate, sodium stearyl fumarate, a polysorbate, e.g., polysorbate 80, docusate sodium, or any combination thereof. In embodiments, the total concentration of the wetting agent in the solid pharmaceutical composition can be from 0.01 wt % to 2.5 wt %, from 0.05 wt % to 1.0 wt %, or from 0.1 wt % to 0.5 wt %. In an embodiment, the wetting agent is present in the intragranular solid composition. In an embodiment, the wetting agent is sodium lauryl sulfate.

In an embodiment, the erdafitinib solid pharmaceutical composition does not include one or more wetting agents.

### Disintegrants

Pharmaceutical excipients for the erdafitinib solid pharmaceutical composition may include one or more disintegrants. The one or more disintegrants can be present in the solid pharmaceutical composition in the intragranular solid composition, the extragranular solid composition, or both the intragranular and extragranular solid composition. In an embodiment, the disintegrant is present in the intragranular solid composition. In an embodiment, the disintegrant is present in the intragranular solid composition and said intragranular solid composition is prepared by roller compaction.

In exemplary embodiments, the disintegrant can comprise or can be selected independently from a functionalized polysaccharide or a crosslinked polymer. For example, in an aspect, the disintegrant can comprise or can be selected from, for example (a) a cellulose which is functionalized with methoxy-, 2-hydroxypropoxy-, or carboxymethoxy-moieties, a salt thereof, or a combination thereof, (b) a carboxymethylated starch, or (c) a crosslinked polymer.

In embodiments, the disintegrant can comprise or can be selected independently from hydroxypropyl methylcellulose, low-substituted hydroxypropylcellulose, crospovidone (crosslinked polyvinylpyrrolidone), croscarmellose sodium (cross-linked sodium carboxymethylcellulose), sodium starch glycolate, or any combination thereof.

When present, the disintegrant can be present in a range of concentrations. In embodiments, the total concentration of the disintegrant in the solid pharmaceutical composition can be from 0.1 wt % to 3 wt %, from 0.5 wt % to 2.5 wt %, from 1 wt % to 2 wt %, or about 1.5 wt %.

In an embodiment, the erdafitinib solid pharmaceutical composition does not include one or more disintegrants.

### Diluents or Fillers

Pharmaceutical excipients for the erdafitinib solid pharmaceutical composition may include one or more diluents. The one or more diluents can be present in the solid pharmaceutical composition as a component of the intragranular solid composition, the extragranular solid composition, or both the intragranular and extragranular solid composition.

In exemplary embodiments, diluents can comprise or can be selected from a sugar, starch, microcrystalline cellulose, a sugar alcohol, a hydrogen phosphate salt, a dihydrogen phosphate salt, a carbonate salt, or combinations thereof. In an aspect, diluents can comprise or can be selected from lactose, dextrin, mannitol, sorbitol, starch, microcrystalline cellulose, silicified microcrystalline cellulose, dibasic calcium phosphate, anhydrous dibasic calcium phosphate, calcium carbonate, sucrose, or any combination thereof.

In embodiments, the total concentration of the diluent in the solid pharmaceutical composition can be from 10 wt % to 60 wt %, from 10 wt % to 50 wt %, from 10 wt % to 40 wt %, from 12 wt % to 30 wt %, from 15 wt % to 25 wt %, or from 18 wt % to 22 wt %, or from 20 wt % to 40 wt %, or from 20 wt % to 30 wt %, or from 25 wt % to 30 wt %. For example, in some aspects, the diluent can comprise or can be selected from microcrystalline cellulose in a concentration of from 15 wt % to 25 wt %, or from 20 wt % to 22 wt %, or from 15 wt % to 20 wt %. In a further aspect, the diluent can comprise or can be selected from anhydrous dibasic calcium phosphate in a concentration of from 18 wt % to 20 wt %. In a further aspect, the diluent can comprise or can be anhydrous dibasic calcium phosphate in a concentration of about 19 wt %. In a further aspect, the diluent can comprise or can be anhydrous dibasic calcium phosphate in a concentration of about 19 wt %, which is present in the extragranular solid composition. In a further aspect, the diluent can comprise or can be selected from silicified microcrystalline cellulose in a concentration of from 10 wt % to 20 wt %, or from 10 wt % to 15 wt %, or from 10 wt % to 12 wt %. For example, the diluent can comprise silicified microcrystalline cellulose in a concentration of about 10.75 wt % or 11.75 wt % of the solid composition. For example, the diluent can comprise silicified microcrystalline cellulose in a concentration of about 10.75 wt % or 11.75 wt % of the solid composition and is present in the extragranular composition. For example, the diluent can comprise silicified microcrystalline cellulose in a concentration of about 10.75 wt % of the solid composition and is present in the extragranular composition. For example, the diluent can comprise silicified microcrystalline cellulose in a concentration of about 11.75 wt % of the solid composition and is present in the extragranular composition. In a further aspect, the diluent does not include silicified microcrystalline cellulose. In a further aspect, the diluent may comprise microcrystalline cellulose and silicified microcrystalline cellulose. In a further aspect, the diluent may comprise microcrystalline cellulose or silicified microcrystalline cellulose. In a further aspect, the diluent may comprise microcrystalline cellulose in a concentration of about 10 wt %. In a further aspect, the diluent may comprise microcrystalline cellulose in a concentration of about 10 wt %, which is present in the intragranular composition. For example, the microcrystalline cellulose can be present in the solid pharmaceutical composition as a filler and/or as a binder at a concentration of about 17.5 wt %. For example, the microcrystalline cellulose can be present in the solid pharmaceutical composition at a concentration of about 17.5% wt % of the solid composition and is present in the intragranular solid composition and extragranular solid composition. For example, the microcrystalline cellulose can be present in the solid pharmaceutical composition as a filler in the intragranular composition, at a concentration of about 10 wt % of the solid composition, and can be present in the solid pharmaceutical composition as a binder in the extragranular composition, at a concentration of about 7.5 wt % of the solid composition.

It will be appreciated by the person of ordinary skill that some of the diluents/fillers disclosed herein may also function as binders in the pharmaceutical composition. Accordingly, some compounds or materials may be described herein as providing a binder function and providing a diluent/filler function.

### Glidants

Pharmaceutical excipients for the erdafitinib solid pharmaceutical composition may include one or more glidants. The one or more glidants can be present in the solid pharmaceutical composition as a component of the intragranular solid composition, the extragranular solid composition, or both the intragranular and extragranular solid composition. In an aspect, the glidant is present in the extragranular solid composition. As used in this disclosure, a glidant refers to a pharmaceutical excipient which improves or optimizes the particle flow properties of the granulated or powdered tablet components in particle form by decreasing the interaction, attraction, cohesion, or friction between particles. Pharmaceutically acceptable glidants are non-toxic and pharmacologically inactive substances. Further, the glidants can be water soluble or water insoluble.

In an aspect, glidants can include or can be selected from colloidal silicon dioxide, colloidal anhydrous silicon dioxide, talc, or any combination thereof. In embodiments, the total concentration of the glidant in the solid pharmaceutical composition can be from 0.01 wt % to 5 wt %, 0.05 wt % to 3 wt %, 0.1 wt % to 1 wt %, or about 0.2 wt %, or about 0.25 wt %, or about 0.3 wt %, about 0.35 wt %, or about 0.4 wt %, or about 0.45 wt % or about 0.5 wt %. In an embodiment, the glidant is colloidal silicon dioxide. In some embodiments, the glidant is colloidal silicon dioxide at about 0.5 wt % of the solid composition. In some embodiments, the glidant is colloidal silicon dioxide at about 0.5 wt % of the solid composition, and is present in the extragranular composition. In some embodiments, the glidant is colloidal silicon dioxide at about 0.25 wt % of the solid composition. In some embodiments, the glidant is colloidal silicon dioxide at about 0.25 wt % of the solid composition, and is present in the extragranular composition.

### Lubricants

Pharmaceutical excipients for the erdafitinib solid pharmaceutical composition may include one or more lubricants. The one or more lubricants can be present in the solid pharmaceutical composition as a component of the intragranular solid composition, the extragranular solid composition, or both the intragranular and extragranular solid composition. In an aspect, the lubricant is present in the extragranular solid composition. In an aspect, the lubricant is present in the intragranular solid composition, and said intragranular solid composition is prepared by roller compaction. As used in this disclosure, a lubricant refers to a pharmaceutical excipient added to a tablet formulation which reduces friction at the tablet's surface. In embodiments, the lubricant can reduce friction between a tablet's surface and processing equipment, e.g., between a tablet's surface and the wall of a die cavity in which a tablet is formed. Therefore, a lubricant can reduce friction between a die wall and the granules of the formulation as the tablet is formed and ejected. Pharmaceutically acceptable lubricants are non-toxic and pharmacologically inactive substances. Further, the lubricants can be water soluble or water insoluble.

In an aspect, the lubricant can comprise or can be selected from, for example, a fatty acid, a fatty acid salt, a fatty acid ester, talc, a glyceride ester, a metal silicate, or any combination thereof. In embodiments, the lubricant can comprise or can be selected from magnesium stearate, stearic acid, magnesium silicate, aluminum silicate, isopropyl myristate, sodium oleate, sodium stearoyl lactate, sodium stearoyl fumarate, titanium dioxide, or combinations thereof. Examples of lubricants include but are not limited to leucine, sodium lauryl sulfate, sucrose stearate, boric acid, sodium acetate, sodium oleate, sodium stearyl fumarate, and PEG. In another aspect, the total concentration of the lubricant in the solid pharmaceutical composition can be from 0.05 wt % to 5 wt %, 0.1 wt % to 3 wt %, 1 wt % to 2 wt %, or about 1.5 wt %. In an embodiment, the lubricant is magnesium stearate. In some embodiments, the lubricant is magnesium stearate, and is present in the intragranular composition or the extragranular composition. In some embodiments, the lubricant is magnesium stearate, and is present in the intragranular composition and the extragranular composition. In some embodiments, the lubricant is magnesium stearate at about 1.5 wt % of the solid composition. In some embodiments, the lubricant is magnesium stearate at about 1.5 wt % of the solid composition, and is present in the intragranular composition. In some embodiments, the lubricant is magnesium stearate at about 1.5 wt % of the solid composition, and is present in the extragranular composition. In some embodiments, the lubricant is magnesium stearate at about 1.5 wt % of the solid composition, and is present in the intragranular composition and the extragranular composition.

### Formulation Development

Provided herein are erdafitinib formulations, in particular erdafitinib tablets, that (a) comprise a high erdafitinib drug load, such as ranging from 40 wt % to 70 wt %, or from 40 wt % to 60 wt %, or from 45 wt % to 55 wt %, or about 50 wt %, or ranging from 45 wt % to 55 wt %, or about 50 wt %, (b) provide for an acceptable chemical stability of erdafitinib, (c) support high production speeds for tablet production, e.g., on an industrial scale, in particular tablets having a length (L) that exceeds its diameter (D) so that the tablet has an aspect ratio (L:D) of greater than 1:1, in particular such tablets having a cylindrical diameter of from 1.0 mm to 3.2 mm, or from 1.5 mm to 3.1 mm or from 2.0 mm to 2.7 mm or from 2.5 mm to 2.7 mm, in particular on an industrial scale, in particular for minitablets, (d) provide a tablet that is sufficiently robust physically, in particular is suitable for being included in a drug delivery system as described herein, in particular a permeation system, and/or (e) exhibits desired disintegration and/or dissolution properties.

Erdafitinib formulations with a range of excipient combinations, both intragranular and extragranular, are provided in Table 1 in the Examples which sets out Formula 4A, Formula 4B, Formula 4C, and Formula 4D. Further erdafitinib formulations with a range of excipient combinations, are provided in Table 3 and in the Examples which set forth formulations 3.2, 3.3, 3.4, and 4.1.

Provided herein are erdafitinib solid formulations, in particular erdafitinib minitablets, in particular with a high erdafitinib drug load, such as ranging from 40 wt % to 70 wt %, or from 40 wt % to 60 wt %, or from 45 wt % to 55 wt %, or about 50 wt %, or ranging from 45 wt % to 55 wt %, or about 50 wt %. In an embodiment the tablets are obtainable by a process that comprises fluid bed granulation. In an embodiment the tablets are obtainable by a process that comprises roller compaction. In an embodiment the intragranular solid composition comprises a cyclodextrin, in particular hydroxypropyl-beta-cyclodextrin. In an embodiment the formulation does not comprise mannitol in the intragranular solid composition. In an embodiment, the intragranular solid composition does not comprise a water soluble filler. In an embodiment, the formulation comprises a water insoluble filler, such as for example microcrystalline cellulose.

In an embodiment, there is provided a fluid bed granulation process for making granules comprising erdafitinib and hydroxypropyl-beta-cyclodextrin. In an aspect, the process does not comprise using a water soluble filler such as mannitol.

Provided herein are erdafitinib solid formulations, in particular erdafitinib minitablets, in particular with a high erdafitinib drug load, such as ranging from 45 wt % to 55 wt %, or about 50 wt % comprising vinylpyrrolidinone-vinyl acetate copolymer and microcrystalline cellulose, in particular in a weight ratio ranging from 1:99 to 99:1, or from 5:95 to 95:5, or from 10:90 to 90:10, or from 20:80 to 80:20, or from 30:70 to 70:30, or from 40:60 to 60:40 or 50:50. It was unexpectedly found that ejection forces during tableting, in particular tableting of mini-tablets, such as those described herein, were reduced in the presence of this mixture. It was found that a powder formulation comprising such a mixture had good flow properties. In an aspect the formulation further comprises hydroxypropyl-beta-cyclodextrin. In an aspect the formulation does not comprise mannitol.

In an embodiment, a process is provided for making tablets, in particular minitablets as described herein, wherein the powder blend to be tableted comprises vinylpyrrolidinone-vinyl acetate copolymer and microcrystalline cellulose, in particular in a weight ratio ranging from 1:99 to 99:1, or from 5:95 to 95:5, or from 10:90 to 90:10, or from 20:80 to 80:20, or from 30:70 to 70:30, or from 40:60 to 60:40 or 50:50. In an aspect, there is provided a process for making tablets, in particular minitablets as described herein, wherein the powder blend to be tableted comprises erdafitinib, vinylpyrrolidinone-vinyl acetate copolymer and microcrystalline cellulose, in particular wherein the weight ratio of vinylpyrrolidinone-vinyl acetate copolymer and microcrystalline cellulose ranges from 1:99 to 99:1, or from 5:95 to 95:5, or from 10:90 to 90:10, or from 20:80 to 80:20, or from 30:70 to 70:30, or from 40:60 to 60:40 or 50:50. In an aspect, the powder blend to be tableted further comprises hydroxypropyl-beta-cyclodextrin. In an aspect, the powder blend to be tableted does not comprise mannitol.

Provided herein are erdafitinib solid formulations, in particular erdafitinib powder formulations or erdafitinib minitablets, in particular with a high erdafitinib drug load, such as ranging from 40 wt % to 70 wt %, or from 40 wt % to 60 wt %, or from 45 wt % to 55 wt %, or about 50 wt %, or ranging from 45 wt % to 55 wt %, or about 50 wt % having a low content of fine particles, such as for example a content of fine particles below 20%, or below 10%, or below 5%, or about or below 3%, or about or below 2%. Fine particles can increase the ejection forces during tableting, especially during tableting of minitablets as described herein, in particular when tableting at a high speed, such as for example 2500 tablets/minute.

In an embodiment, provided herein is a formulation, in particular a tablet or minitablet, comprising erdafitinib, in particular with a high erdafitinib drug load, such as ranging from 40 wt % to 70 wt %, or from 40 wt % to 60 wt %, or from 45 wt % to 55 wt %, or about 50 wt %, or ranging from 45 wt % to 55 wt %, or about 50 wt %, hydroxypropyl-beta-cyclodextrin, vinylpyrrolidinone-vinyl acetate copolymer and microcrystalline cellulose. In an aspect, the formulation further comprises meglumine. In an aspect, the formulation does not comprise mannitol. In an aspect the formulation further comprises at least one or all of a glidant, such as for example colloidal silica, a lubricant, such as for example magnesium stearate, a binder such as for example a cellulose derivative, such as hydroxypropyl methylcellulose, a filler, such as for example silicified microcrystalline cellulose.

In an embodiment, provided herein is a formulation, in particular a tablet or minitablet, comprising erdafitinib, in particular with a high erdafitinib drug load, such as ranging from 40 wt % to 70 wt %, or from 40 wt % to 60 wt %, or from 45 wt % to 55 wt %, or about 50 wt %, or ranging from 45 wt % to 55 wt %, or about 50 wt %, hydroxypropyl-beta-cyclodextrin, vinylpyrrolidinone-vinyl acetate copolymer and microcrystalline cellulose. In an aspect, the formulation further comprises at least one or all of a glidant, such as for example colloidal silica, a lubricant, such as for example magnesium stearate, a binder such as for example a cellulose derivative, such as hydroxypropyl methylcellulose, a filler, such as for example silicified microcrystalline cellulose. In an aspect, the formulation does not comprise a stabilizer, such as meglumine. In an aspect, the formulation does not comprise mannitol.

In an embodiment, the formulation is Formula 4A. In an embodiment, the formulation is Formula 4B. In an embodiment, the formulation is Formula 4C. In an embodiment, the formulation is Formula 4D.

Accordingly, Formula 4D formulation is encompassed by this disclosure, in which the solid pharmaceutical composition includes: (a) 50 wt % erdafitinib free base; (b) 10 wt % hydroxypropyl-beta-cyclodextrin; (c) 1 wt % meglumine; (d) 17.5 wt % microcrystalline cellulose; (e) 10.75 wt % silicified microcrystalline cellulose; (f) 7.5 wt % vinylpyrrolidone-vinyl acetate copolymer; (g) 0.25 wt % colloidal silicon dioxide; (h) 1.5 wt % hydroxypropyl methylcellulose; and (i) 1.5 wt % magnesium stearate, wherein these weight percentages are relative to the entire solid pharmaceutical composition. In an aspect, this formulation can be prepared by a process comprising (a) preparing an intragranular solid composition by a fluid bed granulation process, the intragranular solid composition consisting essentially of: (i) erdafitinib free base in a concentration of 50 wt % of the solid pharmaceutical composition; (ii) hydroxypropyl-beta-cyclodextrin in a concentration of 10 wt % of the solid pharmaceutical composition; (iii) meglumine in a concentration of 1 wt % of the solid pharmaceutical composition; (iv) microcrystalline cellulose in a concentration of 10 wt % of the solid pharmaceutical composition; and (v) hydroxypropyl methylcellulose in a concentration of 1.5 wt % of the solid pharmaceutical composition; (b) combining the intragranular solid composition with extragranular components to form a blend, wherein the extragranular components consist essentially of: (i) microcrystalline cellulose in a concentration of 7.5 wt % of the solid pharmaceutical composition; and (ii) vinylpyrrolidone-vinyl acetate copolymer in a concentration of 7.5 wt % of the solid pharmaceutical composition; (iii) silicified microcrystalline cellulose in a concentration of 10.75 wt % of the solid pharmaceutical composition; (iv) colloidal silicon dioxide in a concentration of 0.25 wt % of the solid pharmaceutical composition; and (iv) magnesium stearate in a concentration of 1.5 wt % of the solid pharmaceutical composition; and (c) tableting the blend to form of a solid pharmaceutical composition in the form of mini-tablets. In an embodiment, the tablet comprises 11.5 mg of erdafitinib.

Accordingly, Formula 4C formulation is encompassed by this disclosure, in which the solid pharmaceutical composition includes: (a) 50 wt % erdafitinib free base; (b) 10 wt % hydroxypropyl-beta-cyclodextrin; (c) 1 wt % meglumine; (d) 1.5 wt % hydroxypropyl methylcellulose; (e) 21.0 wt % mannitol; (f) 0.25 wt % sodium lauryl sulfate; (g) 7.25 wt % microcrystalline cellulose; (h) 7.25 wt % vinylpyrrolidone-vinyl acetate copolymer; (i) 0.25 wt % colloidal silicon dioxide; and (j) 1.50 wt % magnesium stearate, wherein these weight percentages are relative to the entire solid pharmaceutical composition. In an aspect, this formulation may be prepared by a process comprising (a) preparing an intragranular solid composition by a fluid bed granulation process; (b) combining the intragranular solid composition with extragranular components to form a blend; and (c) tableting the blend to form a solid pharmaceutical composition in the form of mini-tablets, in which the intragranular and the extragranular components are set out in the Examples in Table 1. In an embodiment, the tablet comprises 11.5 mg of erdafitinib.

Accordingly, Formula 4B formulation is encompassed by this disclosure, in which the solid pharmaceutical composition includes (a) 50 wt % erdafitinib free base; (b) 10 wt % hydroxypropyl-beta-cyclodextrin; (c) 1 wt % meglumine; (d) 24.5 wt % microcrystalline cellulose; (e) 6.0 wt % silicified microcrystalline cellulose; (f) 6.0 wt % vinylpyrrolidone-vinyl acetate copolymer; (g) 0.5 wt % colloidal silicon dioxide; and (h) 2.0 wt % magnesium stearate, wherein these weight percentages are relative to the entire solid pharmaceutical composition. In an aspect, this formulation may be prepared by a process comprising (a) preparing an intragranular solid composition by a fluid bed granulation process; (b) combining the intragranular solid composition with extragranular components to form a blend; and (c) tableting the blend to form of a solid pharmaceutical composition in the form of mini-tablets, in which the intragranular and the extragranular components are set out in the Examples in Table 1. In an aspect, this formulation may be prepared by a process comprising (a) preparing an intragranular solid composition by a roller compaction process; (b) combining the intragranular solid composition with extragranular components to form a blend; and (c) tableting the blend to form of a solid pharmaceutical composition in the form of mini-tablets, in which the intragranular and the extragranular components are set out in the Examples in Table 1. In an embodiment, the tablet comprises 11.5 mg of erdafitinib.

Accordingly, Formula 4A formulation is encompassed by this disclosure, in which the solid pharmaceutical composition includes (a) 50 wt % erdafitinib free base; (b) 10 wt % hydroxypropyl-beta-cyclodextrin; (c) 1 wt % meglumine; (d) 10 wt % microcrystalline cellulose; (e) 19 wt % anhydrous dibasic calcium phosphate; (f) 8 wt % vinylpyrrolidone-vinyl acetate copolymer; (g) 0.5 wt % colloidal silicon dioxide; and (h) 1.50 wt % magnesium stearate, wherein these weight percentages are relative to the entire solid pharmaceutical composition. In an aspect, this formulation may be prepared by a process comprising (a) preparing an intragranular solid composition by a fluid bed granulation process; (b) combining the intragranular solid composition with extragranular components to form a blend; and (c) tableting the blend to form a solid pharmaceutical composition in the form of mini-tablets, in which the intragranular and the extragranular components are set out in the Examples in Table 1. In an aspect, this formulation may be prepared by a process comprising (a) preparing an intragranular solid composition by a roller compaction process; (b) combining the intragranular solid composition with extragranular components to form a blend; and (c) tableting the blend to form a solid pharmaceutical composition in the form of mini-tablets, in which the intragranular and the extragranular components are set out in the Examples in Table 1. In an embodiment, the tablet comprises 11.5 mg of erdafitinib.

Accordingly, Formulation 4.1 is encompassed by the disclosure, in which the solid pharmaceutical composition includes: (a) 50 wt % erdafitinib free base; (b) 10 wt % hydroxypropyl-beta-cyclodextrin; (c) 17.5 wt % microcrystalline cellulose; (d) 11.75 wt % silicified microcrystalline cellulose; (e) 7.5 wt % vinylpyrrolidone-vinyl acetate copolymer; (f) 0.25 wt % colloidal silicon dioxide; (g) 1.5 wt % hydroxypropyl methylcellulose; and (h) 1.5 wt % magnesium stearate, wherein these weight percentages are relative to the entire solid pharmaceutical composition. In an aspect, this formulation can be prepared by a process comprising (a) preparing an intragranular solid composition by a fluid bed granulation process, the intragranular solid composition consisting essentially of: (i) erdafitinib free base in a concentration of 50 wt % of the solid pharmaceutical composition; (ii) hydroxypropyl-beta-cyclodextrin in a concentration of 10 wt % of the solid pharmaceutical composition; (iii) microcrystalline cellulose in a concentration of 10 wt % of the solid pharmaceutical composition; and (iv) hydroxypropyl methylcellulose in a concentration of 1.5 wt % of the solid pharmaceutical composition; (b) combining the intragranular solid composition with extragranular components to form a blend, wherein the extragranular components consist essentially of: (i) microcrystalline cellulose in a concentration of 7.5 wt % of the solid pharmaceutical composition; and (ii) vinylpyrrolidone-vinyl acetate copolymer in a concentration of 7.5 wt % of the solid pharmaceutical composition; (iii) silicified microcrystalline cellulose in a concentration of 11.75 wt % of the solid pharmaceutical composition; (iv) colloidal silicon dioxide in a concentration of 0.25 wt % of the solid pharmaceutical composition; and (iv) magnesium stearate in a concentration of 1.5 wt % of the solid pharmaceutical composition; and (c) tableting the blend to form of a solid pharmaceutical composition in the form of mini-tablets. In an embodiment, the tablet comprises 11.5 mg of erdafitinib.

### Diffusion-Based Drug Delivery Systems

Drug delivery systems particularly suitable for the effective release of drug formulations containing erdafitinib, such as those described in detail above or hereinafter, are described herein. These particular systems have been developed wherein, instead of an osmotic drug release mechanism, drug release is controlled by drug diffusion through a drug-permeable polymer component defining part of the system housing.

In certain embodiments, the system includes a drug-permeable polymer component or portion that forms a portion of the housing. For example, the drug-permeable component or portion of the system may be a portion of the housing formed of a material distinct from the remaining portion of housing (e.g., a strip or multiple strips of material extending along at least a portion of the length of the housing), such that the size, shape (e.g., arc angle), thickness, and material properties of the drug-permeable wall structure may be selected to achieve the desired drug release rate. In certain embodiments, the drug permeable portion, the drug impermeable portion, or both the drug permeable and impermeable portions are formed of thermoplastic polyurethane compositions, to provide (i) controlled diffusion of the drug from the system, (ii) desired mechanical properties (e.g., able to be straightened for insertion/removal, soft enough to be well-tolerated while indwelling, tubing remains intact with small compressions/extensions, elastic deformability in response to detrusor muscle contraction (compliancy)), (iii) a system that may be thermally shape set to have a desired retention shape, and/or (iv) a system which may be manufactured in a coextrusion process.

In some embodiments, the drug permeable portion is permeable to erdafitinib free base. In some embodiments, the drug permeable portion is permeable to erdafitinib free base and erdafitinib free base formulated with HP-β-CD. In some embodiments, the drug permeable portion is permeable to erdafitinib free base, erdafitinib HCl salt, and erdafitinib free base formulated with HP-3-CD. In some embodiments of any of the foregoing, the material of the drug permeable portion is an aliphatic polyether-based TPU. In some embodiments of the foregoing, the material of the drug permeable portion is an aliphatic polyether-based TPU which is Lubrizol Tecophilic HP-60D-35 or HP-93A-100.

In some embodiments, the drug permeable portion is permeable to erdafitinib free base formulated with HP-β-CD. In some embodiments, the drug permeable portion is permeable to erdafitinib free base formulated with HP-3-CD, and is impermeable or practically impermeable to erdafitinib free base formulated without HP-β-CD. In some embodiments of any of the foregoing, the material of the drug permeable portion is an aliphatic polyether-based TPU. In some embodiments of the foregoing, the material of the drug-permeable portion is Lubrizol Tecoflex EG-80A.

Exemplary materials for the drug-permeable portion (e.g., the “stripe” material of the permeation system) include, but are not limited to, aliphatic polyether-based thermoplastic polyurethanes (TPUs) such as Lubrizol Tecophilic HP-60D-35, Tecophilic HP-93A-100, and Tecoflex EG-80A. In some embodiments, the material of the drug-permeable portion is Lubrizol Tecophilic HP-60D-35, Tecophilic HP-93A-100, or Tecoflex EG-80A. In some embodiments, the material of the drug-permeable portion is Lubrizol Tecoflex EG-80A. In some embodiments, the drug is erdafitinib free base, and the material of the drug-permeable portion is Lubrizol Tecophilic HP-60D-35 or Tecophilic HP-93A-100. In some embodiments, the drug is erdafitinib free base, the drug is formulated with HP-β-CD, and the material of the drug-permeable portion is Lubrizol Tecophilic HP-60D-35, Tecophilic HP-93A-100, or Tecoflex EG-80A. In some embodiments, the drug is erdafitinib free base, the drug is formulated with HP-β-CD, and the material of the drug-permeable portion is Lubrizol Tecoflex EG-80A. In some embodiments, the drug is erdafitinib HCl salt, and the material of the drug-permeable portion is Lubrizol Tecophilic HP-60D-35 or Tecophilic HP-93A-100.

Exemplary materials for the drug-impermeable portion (e.g., the “base” material of the permeation system) include, but are not limited to, silicone elastomer materials such as NuSil MED-4750; TPUs such as Lubrizol Carbothane Aliphatic PC-3575A, Tecothane Soft AR-62A, AR-75A-B20, AC-4075A-B20, Carbothane Aromatic AC-4075A, Tecothane TT-1074A, Tecoflex EG-80A; and ethylene vinyl acetate such as 3M CoTran 9712. In some embodiments, the material of the drug-impermeable portion is selected from MED-4750, PC-3575A, PC-3575A, AR-62A, AR-75A-B20, AC-4075A-B20, AC-4075A, TT-1074A, EG-80A, and CoTran 9712. In some embodiments, the material of the drug-impermeable portion is selected from MED-4750, PC-3575A, PC-3575A, AR-62A, AR-75A-B20, AC-4075A-B20, AC-4075A, TT-1074A, and CoTran 9712. In some embodiments, the material of the drug-impermeable portion is AR-75A-B20. In some embodiments, the material of the drug-impermeable portion is AC-4075A-B20.

In some embodiments, the material of the drug-permeable portion is EG-80A, and the material of the drug-impermeable portion is AR-75A-B20. In some embodiments, the material of the drug-permeable portion is EG-80A, and the material of the drug-impermeable portion is AC-4075A-B20.

It is to be understood that Lubrizol Tecophilic HP series materials are aliphatic polyether-based TPUs formulated to absorb equilibrium water contents of up to 100% of the weight of dry resin, designed for extrusion but also processable by injection molding. HP-60D-35 has a shore hardness of about 42D (ASTM D2240), specific gravity of about 1.12 (ASTM D792), flexural modulus (psi) of 4000 (ASTM D790), ultimate tensile (psi) of about 7,800 dry and 4900 wet (ASTM D412), ultimate elongation (%) of about 450 dry and 390 wet (D412); and water absorption (% by Lubrizol Method) of about 35. HP-93A-100 has a shore hardness of about 83A (ASTM D2240), specific gravity of about 1.13 (ASTM D792), flexural modulus (psi) of 2900 (ASTM D790), ultimate tensile (psi) of about 2200 dry and 1400 wet (ASTM D412), ultimate elongation (%) of about 1040 dry and 620 wet (D412); and water absorption (% by Lubrizol Method) of about 100.

It is to be understood that Lubrizol Tecoflex materials are aliphatic polyether-based TPUs processable by extrusion and injection molding. EG-80A has a shore hardness of about 72A (ASTM D2240), specific gravity of about 1.04 (ASTM D792), flexural modulus (psi) of 1,000 (ASTM D790), ultimate tensile (psi) of about 5,800 (ASTM D412), ultimate elongation (%) of about 660 (D412); tensile modulus (psi) of about 300 at 100% elongation, about 500 at 200% elongation, and about 800 at 300% elongation (ASTM D412); and mold shrinkage (in/in) of about 0.008-0.012 (ASTM D955).

It is to be understood that Lubrizol Aromatic Carbothane AC series materials are radiopaque (20% BaSO₄ filled) polycarbonate-based aromatic TPUs, processable by extrusion or injection molding. AC-4075A-B20 has a shore hardness of about 78A (ASTM D2240), specific gravity of about 1.38 (ASTM D792), ultimate tensile (psi) of about 8300 (ASTM D412), ultimate elongation (%) of about 400 (D412); tensile modulus (psi) of about 560 at 100% elongation, about 1300 at 200% elongation, and about 3400 at 300% elongation (ASTM D412); flexural modulus (psi) of about 1800, Vicat temperature (° C.) of about 55, and mold shrinkage (in/in) (1″×0.25″×6″ bar) of about 0.011 (ASTM D955).

It is to be understood that Lubrizol Tecothane Soft materials are aromatic polyester hydrocarbon-based TPUs, processable by extrusion or injection molding. AR-75A has a shore hardness of about 79A (ASTM D785), a specific gravity of about 1.03 (ASTM D792), ultimate tensile (psi) of about 2000 (ASTM D412), ultimate elongation (%) of about 530 (ASTM D412), tensile modulus (psi) of about 730 at 100% elongation, about 1000 at 200% elongation, and about 1300 at 300% elongation (ASTM D412); flexural modulus (psi) of about 2500 (ASTM 790); Vicat softening point (° C.) of about 75; and mold shrinkage (in/in) (1″×0.25″×6″ bar) of about 0.08 (ASTM D955). AR-75A-B20 is 20% BaSO₄ filled AR-75A, and can be manufactured, for example, by Compounding Solutions.

It is to be further understood that abovementioned test results for the Lubrizol Tecophilic HP. Tecoflex, Aromatic Carbothane AC, and Tecothane Soft materials are approximated based on small samples of TPU; therefore the properties of these materials may exhibit slight variation from the properties listed herein.

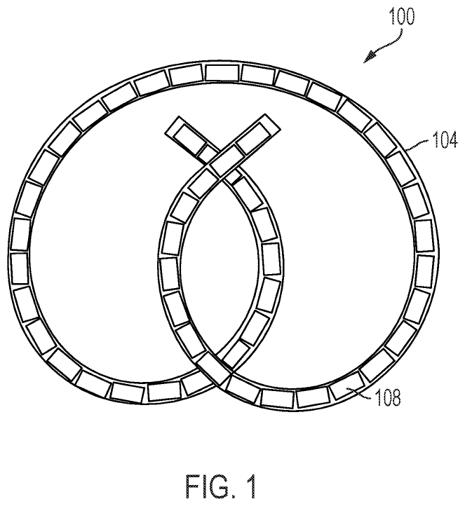

In one aspect, as shown in FIG. 1, a drug delivery system 100 is provided that includes a tubular housing having a drug reservoir lumen 106 bounded by a wall structure 104, wherein (i) at least a portion of the wall structure 104 is water permeable, and (ii) at least a portion of the wall structure is permeable to the drug (contained in drug unit 108) such that the drug is releasable in vivo by diffusion through the drug permeable portion of the wall structure 104. In certain embodiments, as discussed in further detail below, the wall structure includes first and second wall structures that together form the housing. As used herein, the phrase “diffusion through the drug permeable portion” (e.g., through the “second wall structure”) refers to the drug being released by passing through the material forming the wall by molecular diffusion, and not by passing through an aperture or open structure extending through that wall.

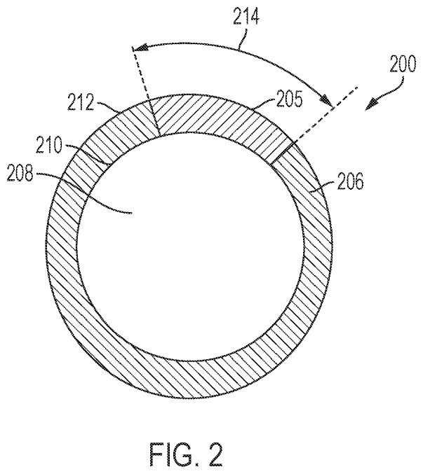

In one aspect, as shown in FIG. 2, a drug delivery system 200 is provided that includes a housing with a first wall structure 206 formed from a first material and a second wall structure 205 formed from a second material, which are adjacent one another and together form a tube defining a drug reservoir lumen 208, wherein (i) the second wall structure 205, or both the first wall structure 206 and the second wall structure 205, are permeable to water, and (ii) the first wall structure 206 is impermeable to the drug and the second wall structure 205 is permeable to the drug, such that the drug is releasable in vivo by diffusion through the second wall structure 205. As used herein, the term “impermeable to the drug” refers to the wall being substantially impermeable to the solubilized drug, such that no substantial amount of the solubilized drug can diffuse therethrough over the therapeutic period in which the system is located in vivo.

In certain embodiments, the tube is cylindrical or another suitable shape or design. As used herein, the term “cylindrical,” when used in reference to the tubular housing, refers to the housing having a substantially cylindrical outer wall. In some embodiments, the system is “closed” and therefore does not include an aperture; drug release is only by diffusion through the second wall structure.

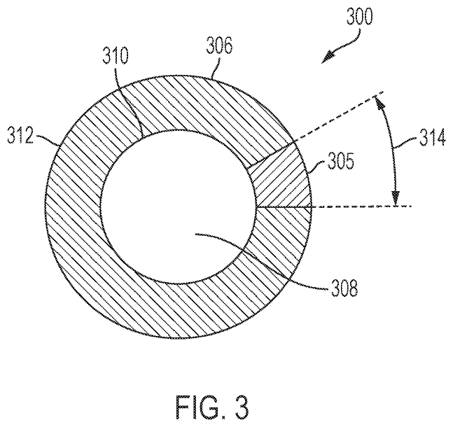

In some embodiments, as shown in FIGS. 2 and 3, the first wall structure 206/306 and the second wall structure 205/305 are adjacent one another and together form a cylindrical tube. For example, such systems may be formed in a coextrusion or 3D-printing process, such that the first and second wall structures are integrally formed. In one embodiment, the coextruded first and second wall structures are thermoplastic polymers possessing the desired properties.

As shown in FIG. 3, the first wall structure 306 and second wall structure 305 together form a cylindrical tube having a lumen 308 in which a drug formulation is contained. The second wall structure 305 is in the form of a longitudinal strip extending along at least a portion of the length of the first wall structure 306 and is permeable to the drug, while the first wall structure 306 is not permeable to the drug. In certain embodiments, multiple drug permeable strips may be used in a single system. In certain embodiments, one permeable strip may be used in a single system. Thus, the size, shape, thickness, and material properties of the second wall structure may be selected to achieve a desired drug release rate.

In a preferred embodiment, as discussed in further detail below, the system is elastically deformable between a low-profile deployment shape (e.g., a relatively straightened shape) suited for insertion through the urethra of a patient and into the patient's bladder and a relatively expanded retention shape (e.g., pretzel shape, bi-oval coil shape, S-shape, etc.) suited for retention within the bladder.

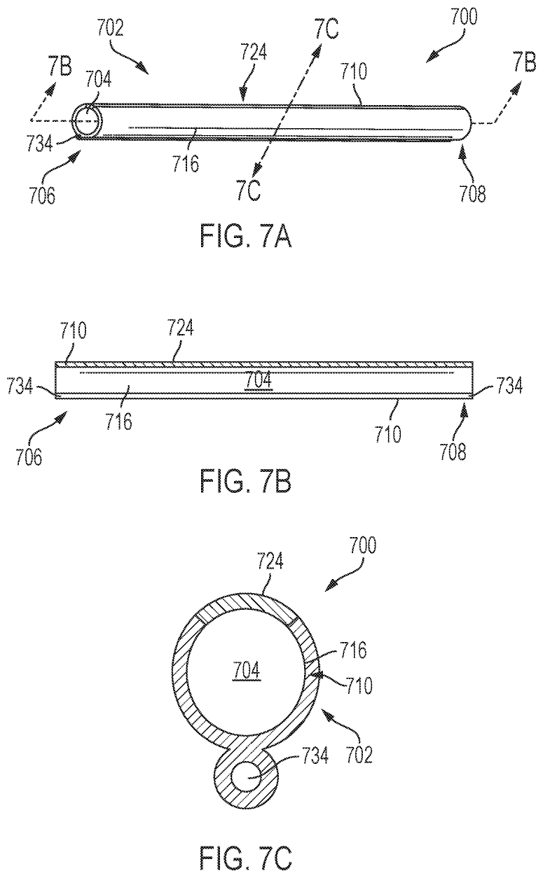

In some embodiments, as shown in FIGS. 7A-7C, the system further includes retention frame lumen 734. In certain embodiments, the retention frame lumen includes an elastic wire, such as a nitinol wire. In certain other embodiments, the retention frame lumen is filled with a shape set elastic polymer.

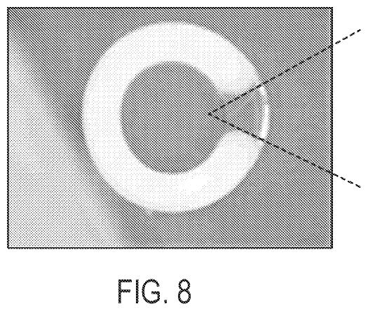

In other embodiments, as shown in FIGS. 1-3 and 8, the system does not include a retention frame lumen or a retention frame or wire. Instead, the material of the housing is configured to be elastically deformable between the straightened shape and the retention shape, in the absence of a retention frame or wire. In certain embodiments, the tubular housing is thermally shape set to have a coiled or other retention shape. Thus, in such embodiments, the design and manufacturing of the system is simplified, and the overall size of the system is minimized (or drug payload may be increased if the size of the system remains constant). In embodiments without a retention frame, the tubular housing material serves the functions of (i) forming the drug reservoir lumen, (ii) controlling drug release, and (iii) retaining the system in the bladder upon deployment.

In one embodiment, as shown in FIGS. 7A-7C, a drug delivery system 700 is provided that includes an elongated, elastic housing 702 having a drug reservoir lumen 704 extending between a first end 706 and a second end 708. The elastic housing 702 is formed of a tubular wall structure 710 that includes a first wall structure 716 and a second wall structure 724 that are adjacent one another and together form a tube defining the drug reservoir lumen 704, wherein (i) the second wall structure 724, or both the first wall structure 716 and the second wall structure 724, are permeable to water, and (ii) the first wall structure 716 is impermeable to the drug and the second wall structure 724 is permeable to the drug, such that the drug is releasable in vivo by diffusion through the second wall structure 724.

In embodiments in which the first and second wall structures together form a cylindrical tube, any suitable end plugs or closures or thermally formed seals may be used to seal the ends of the tube after the drug is loaded. These end plugs/closures ensure that the drug permeable polymer portions forming a portion of the external tube are the only path for drug release.

In some embodiments, as shown in FIGS. 2 and 3, the wall 206, 205/306, 305 has a substantially constant thickness over its circumference. For example, the inner diameter 210/310 and outer diameter 212/312 of the first and second wall structures 206, 205/306, 305 (which together form the cylindrical tube) are the same. In other embodiments, the wall may have a varied thickness over the circumference of the wall.

Thus, for the systems described herein, drug release is controlled by diffusion of the drug through a drug-permeable component defining a portion of the system housing. The drug-permeable wall structure may be located, dimensioned, and have material properties to provide the desired rate of controlled drug diffusion from the system.

The particular material and arc angle of the drug permeable portion or wall structure can be selected to achieve a particular drug release profile, i.e., water and drug permeation rates. As used herein, the phrase “arc angle” refers to the angle dimension of an arc of a circumference of the tube in a cross section normal to a longitudinal axis of the tube.

For example, in certain embodiments, as shown in FIGS. 2 and 3, the second wall structure 205/305 comprises less than 90 percent of a cross sectional area of the tube, in a cross section normal to the longitudinal axis of the tube. In one embodiment, the second wall structure comprises less than 50 percent of a cross sectional area of the tube, in a cross section normal to the longitudinal axis of the tube. In one embodiment, the second wall structure comprises less than 25 percent of a cross sectional area of the tube, in a cross section normal to the longitudinal axis of the tube.

In certain embodiments, as shown at FIGS. 2, 3, 7A-C, and 8, the first and second wall structures that form the tube bounding the drug reservoir lumen are adjacent one another at two interface edges, such that the wall structures collectively form the tube defining the drug reservoir lumen. In these embodiments, the two interface edges are disposed at an arc angle of from about 15 degrees to about 270 degrees of a circumference of the tube in a cross section normal to a longitudinal axis of the tube. As used herein, the phrase “about” with reference to the arc angles of the second wall structure refers to the arc angle plus or minus 3 degrees.

In one embodiment, as shown in FIG. 2, the second wall structure 205 has an arc angle 214 of about 60 degrees of a circumference of the cylindrical tube 200 in the cross-section. In one embodiment, as shown in FIG. 3, the second wall structure 305 has an arc angle 314 of about 30 degrees of a circumference of the cylindrical tube 300 in the cross-section. In one embodiment, the second wall structure has an arc angle of about 15 degrees to about 270 degrees. As will be further described below, in certain embodiments, the second wall structure has an arc angle of about 45 degrees to about 90 degrees, of about 150 degrees to about 270 degrees, or of about 210 degrees to about 270 degrees, such as about 45 degrees, about 90 degrees, about 180 degrees, and about 240 degrees. In certain embodiments, the second wall structure has an arc angle of about 45 degrees, about 90 degrees, about 180 degrees, about 240 degrees, or about 270 degrees.

The second wall structure can be located on the inner curvature (0 degrees), the outer curvature (180 degrees), the top (90 degrees), or in-between, when the system is formed to have a retention shape as shown in FIG. 1. The top (90 degree) location may be preferable when the second wall structure is formed of a material that significantly swells once absorbing water.

Accordingly, tubular systems have been developed which are designed to reduce or control drug release rates without negatively altering the mechanical properties and suitable dimensions for system deployment and tolerability. In some embodiments, the designs reduce drug release rates by reducing the length of the drug permeable regions(s) such that the length runs along only a portion of the overall length of the system. Larger arc angles of the drug permeable region(s) can therefore be employed to tailor drug release rates from the system. Additionally, by decreasing the length of the drug permeable region, a lesser amount of drug permeable material, compared to conventional systems, may be used to effect a reduced drug release rate.

Once the drug is loaded into the drug reservoir lumen, any suitable end plugs or closures or thermally formed seals may be used to seal/close the first and second ends of the drug reservoir lumen. These end plugs/closures ensure that the second material forming a portion of the elastic housing is the sole path for drug release. In certain embodiments, the end plugs are formed of the first material (i.e., the material forming the first wall structure) that is impermeable to the drug.

In the foregoing embodiments, the first material or the first wall structure, the second material or the first wall structure, or both, is formed of a water permeable material. In a preferred embodiment, as described above with reference to the erdafitinib solid formulations, the drug is in a solid form (e.g., a tablet or plurality of tablets) and at least a portion of the tubular body is water permeable to permit in vivo solubilization of the drug while in the drug reservoir lumen. In embodiments, the first material or first wall structure may be the only water permeable portion. In other embodiments both the first and second materials/wall structures may be water permeable.

The material(s) for the wall structures of the present systems can be selected from a variety of suitable thermoplastic polyurethane (TPU)-based materials. In particular, the first material forming the first wall structure (i.e., the material that is impermeable to the drug contained in the drug reservoir) may be a polycarbonate-based aromatic thermoplastic polyurethane (e.g., a CARBOTHANE™ TPU, such as AC-4075A, commercially available from Lubrizol) or an aromatic polyester hydrocarbon-based thermoplastic polyurethane (e.g., a TECOTHANE™ TPU, such as AR-75A, commercially available from Lubrizol). For example, CARBOTHANE polyurethanes are cycloaliphatic polymers and are of the types produced from polycarbonate-based polyols. The general structure of the polyol segment is represented as O—[(CH₂)₆—CO₃]—(CH₂)—O—. AC-4075A has a durometer Shore hardness of 77A, a specific gravity of 1.19, a flexural modulus of 1500 psi, and an ultimate elongation of 400%. AR-75A has a durometer Shore hardness of 79A, a specific gravity of 1.03, a flexural modulus of 2500 psi, and an ultimate elongation of 530%. In particular, the second material forming the second wall structure (i.e., the material that is permeable to the drug contained in the drug reservoir) may be an aliphatic polyether-based thermoplastic polyurethane (e.g., a TECOFLEX™ TPU, such as EG-80A, commercially available from Lubrizol). For example, TECOFLEX polyurethanes are cycloaliphatic polymers and are of the types produced from polyether-based polyols. The general structure of the polyol segment is represented as O—(CH₂—CH₂—CH₂—CH₂)ₓ—O—. EG-80A has a durometer Shore hardness of 72A, a specific gravity of 1.04, a flexural modulus of 1000 psi, and an ultimate elongation of 660%. The TPUs may further include a radiopacity agent, such as barium sulfate, for example, AC-4075A-B20, which is a polycarbonate-based aromatic thermoplastic polyurethane having a 20% loading of barium sulfate.

In one embodiment, an inner diameter of the cylindrical tube may be from about 1.0 mm to about 2.5 mm. In one embodiment, an outer diameter of the cylindrical tube is from about 2.0 mm to about 4.1 mm. In one embodiment, a thickness of the first wall structure, the second wall structure, or both, is from about 0.2 mm to about 1.0 mm.

Thus, as compared to drug delivery systems utilizing a homogenous material (e.g., a blend of permeable and impermeable thermoplastic materials) to form a drug permeable tube, the mechanical properties of a tube utilizing the dual wall structure (e.g., the drug permeable strip embodiments) can be decoupled from the drug release (e.g., diffusion) properties of the tube. For example, in a single material tube, changing the material of tube inherently affects both the mechanical and diffusion properties of the system. Being able to control release rate with stripe angle may have the added benefit of not changing the system outer diameter; in contrast, control by changing wall thickness may become too large to fit through the urethra or too thin to provide the required mechanical strength of the system. Moreover, the drug release properties of a blended polymer may not be readily predictable. In addition, it is often challenging to achieve a truly homogeneous blend when mixing two thermoplastics. Thus, it requires experimentation to modulate drug release rate with such a tubular drug delivery system. In contrast, the dual wall structure described herein may provide enhanced flexibility in tailoring a particular drug release rate from the delivery system.

For use in the bladder, it is important that the system be compliant (e.g., easily flexed, soft feeling) during detrusor muscle contraction in order to avoid or mitigate discomfort and irritation to the patient. Thus, it is noted the durometer of the first and second materials of construction are important, and the proportion of a high durometer material may be limited in constructing a system housing of a given size while keeping it suitably compliant in the bladder. For example, suitable first wall materials, such as TECOTHANE or CARBOTHANE, may have a Shore hardness greater than 70A, such as from 77A to 65D, while suitable second wall materials, such as TECOFLEX, may have a Shore hardness of less than 90A, or less than 80A, such as 72A. In some embodiments, the first material has a Shore hardness value from 70A to 80A while the second material has a Shore hardness value from 70A to 75A. Thus, in certain embodiments, the second wall material has a Shore hardness that is less than the Shore hardness of the first wall material, with both wall materials having a Shore hardness of less than 80A. Accordingly, it can be advantageous to utilize a combination of two different polymeric materials, rather than making the system housing entirely of the water-swelling hydrophilic, drug-permeable second material, to achieve desired mechanical properties of the tube.

In embodiments, the systems described herein are configured to release a therapeutically effective amount of the drug, where the rate of the release of the drug from the drug delivery system is zero order over at least 36 hours. In one embodiment, the rate of the release of the drug from the drug delivery system is essentially zero order over at least 7 days. In embodiments, the system is configured to release a therapeutically effective amount of the drug over a period from 2 days to 6 months, e.g., from 2 days to 90 days, from 7 days to 30 days, or from 7 days to 14 days. Desirably, the rate of the release of the drug from the drug delivery system is zero order over at least 7 days, e.g., from 7 to 14 days, or longer, such as up to 3 months or 90 days. In certain embodiments, the system is configured to begin release of the drug after a lag time. In certain embodiments, the lag time may be at least about 30 minutes, from about 12 hours to about 24 hours, or up to about 2 days. These systems may be effective to release a therapeutically effective amount of the drug for a period of up to 6 months, or up to 3 months (90 days).

As will be discussed in greater detail below, a drug formulation, such as those described throughout this disclosure, is disposed in the drug reservoir lumen defined by the first and second wall structures. In particular preferred embodiments, the drug is an erdafitinib-based pharmaceutical formulation, as described herein. In certain embodiments, the system is configured to release the erdafitinib at an average rate of 1 mg/day to 10 mg/day, depending on the desired treatment regimen. In some embodiments, the system is configured to release the erdafitinib at an average rate of 1 mg/day to 2 mg/day. In such embodiments, the two interface edges may be disposed at an arc angle of 45 degrees to 90 degrees. In some embodiments, the system is configured to release the erdafitinib at an average rate of 4 mg/day to 6 mg/day. In such embodiments, the two interface edges may be disposed at an arc angle of 150 degrees to 270 degrees.

In one embodiment, the system is configured to release the erdafitinib at an average rate of 1 mg/day and the two interface edges are disposed at an arc angle of about 45 degrees. In another embodiment, the system is configured to release the erdafitinib at an average rate of 2 mg/day and the two interface edges are disposed at an arc angle of about 90 degrees. In another embodiment, the system is configured to release the erdafitinib at an average rate of 4 mg/day and the two interface edges are disposed at an arc angle of about 180 degrees. In one embodiment, the system is configured to release the erdafitinib at an average rate of 6 mg/day and the two interface edges are disposed at an arc angle of 240 degrees. In certain embodiments, a release profile of the drug is substantially independent of pH over a pH range of 5 to 7. In certain embodiments, a release profile of the drug is substantially independent of pH over a pH range of 5.5 to 7. In certain embodiments, a release profile of the drug is substantially independent of pH over a pH range of 5.5 to 8. In certain embodiments, the release rates are retained over a period up to 6 months, in particular up to 3 months or 90 days.

In one embodiment, a drug delivery system is provided, which has (i) a housing defining a drug reservoir lumen and a retention frame lumen, (ii) a plurality of tablets comprising erdafitinib disposed in the drug reservoir lumen, and (iii) a nitinol wire form (retention frame) disposed in the retention frame lumen. The drug reservoir lumen is defined/bounded by a first wall structure (base) formed of a first material, which is an aromatic polyester hydrocarbon-based thermoplastic polyurethane, particularly AC-4075A-B20, and a second wall structure (stripe) formed of a second material made of an aliphatic polyether-based thermoplastic polyurethane, particularly EG-80A, where the first and second wall structures are adjacent one another at two interface edges and together forming a tube defining the closed drug reservoir lumen. In an embodiment, the closed drug reservoir lumen contains a plurality of tablets, in particular a plurality of minitablets, in particular erdafitinib minitablets as described herein. In an embodiment, the amount of erdafitinib in the drug reservoir lumen is about 500 mg. In an embodiment, the drug reservoir lumen comprises about 44 erdafitinib minitablets, in particular the erdafitinib tablets as described herein. In one embodiment, the plurality of tablets consists of 44 minitablets, having a total of about 500 mg erdafitinib. In an embodiment, the stripe angle is 90 degrees, and the average release rate of erdafitinib from the system is approximately 2 mg/day. In an embodiment, the stripe angle is 180 degrees, and the average release rate of erdafitinib from the system is approximately 4 mg/day. In an embodiment, the stripe angle is 210-270 degrees, and the average release rate of erdafitinib from the system is approximately 6 mg/day. In an embodiment, the stripe angle is 45 degrees, and the average release rate of erdafitinib from the system is approximately 1 mg/day. In an embodiment, the stripe angle is 90 degrees, and the average release rate of erdafitinib from the system is approximately 2 mg/day. In an embodiment, the stripe angle is 90 degrees, and the average release rate of erdafitinib from the system is approximately 2 mg/day at pH between about 5 and about 6.8, and approximately 1 mg/day at pH about 8. In an embodiment, the stripe angle is 180 degrees, and the average release rate of erdafitinib from the system is approximately 4 mg/day. In an embodiment, the stripe angle is 180 degrees, and the average release rate of erdafitinib from the system is approximately 4 mg/day at pH between about 5 and about 6.8, and approximately 2 mg/day at pH about 8. In an embodiment, the stripe angle is 210-270 degrees, in particular 270 degrees, and the average release rate of erdafitinib from the system is approximately 6 mg/day. In an embodiment, the stripe angle is 210-270 degrees, in particular 270 degrees, and the average release rate of erdafitinib from the system is approximately 6 mg/day at pH between about 5 and about 6.8, and approximately 3 mg/day at pH about 8. In an embodiment, the stripe angle is 45 degrees, and the average release rate of erdafitinib from the system is approximately 1 mg/day. In an embodiment, the stripe angle is 45 degrees, and the average release rate of erdafitinib from the system is approximately 1 mg/day at pH between about 5 and about 6.8, and approximately 0.5 mg/day at pH about 8. In an embodiment, the tablets have Formula 4D as described herein. In an embodiment, the tablets have Formula 4C as described herein. In an embodiment, the tablets have Formula 4B as described herein. In an embodiment, the tablets have Formula 4A as described herein.

### Other Aspects of the Drug Delivery Systems

In certain embodiments, the systems are configured for intravesical insertion and retention in a patient. For example, the systems can be elastically deformable between a relatively low profile (e.g., straightened) shape suited for insertion through a lumen into a body cavity of a patient, such as shown in FIGS. 7A-B, and a relatively expanded retention shape suited to retain the system within the body cavity, e.g., the bladder, such as shown in FIGS. 1, 4, 5, and 6A. The relatively expanded shape may include a pair of overlapping coils, sometime referred to as a “pretzel” shape. In particular embodiments, the ends of the elongated system generally lie within the boundaries of a bi-oval-like shape.

When in the expanded retention shape after deployment in the bladder, for example, the systems may resist excretion in response to the forces of urination or other forces. After drug release, the systems can be removed, for example by cystoscope and forceps, or can be bioerodible, at least in part, to avoid a retrieval procedure.

The system may be loaded with at least one drug in the form of one or more drug units, such as the tablets described throughout this disclosure. Solid drug composition forms, such as tablets, can provide a relatively large drug payload volume to total system volume and potentially enhance stability of the drugs during shipping, storage, before use, or before drug release. Solid drugs, however, may need to be solubilizable in vivo in order to diffuse through the drug-permeable component and into the patient's surrounding tissues or cavity in a therapeutically effective amount. The drug reservoir lumen may hold in an elongated form several of the disclosed drug tablets in an end-to-end serial arrangement. In some embodiments, the system holds from about 10 to 100 cylindrical drug tablets (e.g., 44 tablets), such as mini-tablets, which may be serially loaded in the drug reservoir lumen. In an aspect, the tablets are those as described herein. In an aspect, the tablets are those of Formula 4A. In an aspect, the tablets are those of Formula 4B. In an aspect, the tablets are those of Formula 4C. In an aspect, the tablets are those of Formula 4D.

The systems may be inserted into a patient using a cystoscope or catheter or any other suitable or customized inserter device. Typically, a cystoscope for an adult human has an outer diameter of about 5 mm and a working channel having an inner diameter of about 2.4 mm to about 2.6 mm. In embodiments, a cystoscope may have a working channel with a larger inner diameter, such as an inner diameter of 4 mm or more. Thus, the system may be relatively small in size. For example, when the system is elastically deformed to the relatively straightened shape, the system for an adult patient may have a total outer diameter that is less than about 2.6 mm, such as between about 2.0 mm and about 2.4 mm. In addition to permitting insertion, the relatively small size of the system may also reduce patient discomfort and trauma to the bladder. In one embodiment, the overall configuration of the system promotes in vivo tolerability for most patients. In a particular embodiment, the system is configured for tolerability based on bladder characteristics and design considerations described in U.S. Pat. No. 11,065,426.

Within the three-dimensional space occupied by the system in the retention shape, the maximum dimension of the system in any direction preferably is less than 10 cm, the approximate diameter of the bladder when filled. In some embodiments, the maximum dimension of the system in any direction may be less than about 9 cm, such as about 8 cm, 7 cm, 6 cm, 5 cm, 4.5 cm, 4 cm, 3.5 cm, 3 cm, 2.5 or smaller. In particular embodiments, the maximum dimension of the system in any direction is less than about 7 cm, such as about 6 cm, 5 cm, 4.5 cm, 4 cm, 3.5 cm, 3 cm, 2.5 cm or smaller. In preferred embodiments, the maximum dimension of the system in any direction is less than about 6 cm, such as about 5 cm, 4.5 cm, 4 cm, 3.5 cm, 3 cm, 2.5 cm or smaller. More particularly, the three-dimension space occupied by the system is defined by three perpendicular directions. Along one of these directions the system has its maximum dimension, and along the two other directions the system may have smaller dimensions. For example, the smaller dimensions in the two other directions may be less than about 4 cm, such as about 3.5 cm, 3 cm, 2.5 cm or less. In a preferred embodiment, the system has a dimension in at least one of these directions that is less than 3 cm.

In some embodiments, the system may have a different dimension in at least two of the three directions, and in some cases in each of the three directions, so that the system is non-uniform in shape. Due to the non-uniform shape, the system may be able to achieve an orientation of reduced compression in the empty bladder, which also is non-uniform in shape. In other words, a particular orientation of the system in the empty bladder may allow the system to exert less contact pressure against the bladder wall, making the system more tolerable for the patient.

The overall shape of the system may enable the system to reorient itself within the bladder to reduce its engagement or contact with the bladder wall. For example, the overall exterior shape of the system may be curved, and all or a majority of the exterior or exposed surfaces of the system may be substantially rounded. The system also may be substantially devoid of sharp edges, and its exterior surfaces may be formed from a material that experiences reduced frictional engagement with the bladder wall. Such a configuration may enable the system to reposition itself within the empty bladder so that the system applies lower contact pressures to the bladder wall. In other words, the system may slip or roll against the bladder wall into a lower energy position, meaning a position in which the system experiences less compression.

In one embodiment, the system is generally planar in shape even though the system occupies three-dimensional space. Such a system may define a minor axis, about which the system is substantially symmetrical, and a major axis that is substantially perpendicular to the minor axis. The system may have a maximum dimension in the direction of the major axis that does not exceed about 6 cm, and in particular embodiments is less than 5 cm, such as about 4.5 cm, about 4 cm, about 3.5 cm, about 3 cm, or smaller. The system may have a maximum dimension in the direction of the minor axis that does not exceed about 4.5 cm, and in particular embodiments is less than 4 cm, such as about 3.5 cm, about 3 cm, or smaller. The system is curved about substantially its entire exterior perimeter in both a major cross-sectional plane and a minor cross-sectional plane. In other words, the overall exterior shape of the system is curved and the cross-sectional shape of the system is rounded. Thus, the system is substantially devoid of edges, except for edges on the two flat ends, which are completely protected within the interior of the system when the system lies in a plane. These characteristics enable the system to reorient itself into a position of reduced compression when in the empty bladder.

The system also may be small enough in the retention shape to permit intravesical mobility. In particular, the system when deployed may be small enough to move within the bladder, such as to move freely or unimpeded throughout the entire bladder under most conditions of bladder fullness, facilitating patient tolerance of the system. Free movement of the system also facilitates uniform drug delivery throughout the entire bladder.

The system also may be configured to facilitate buoyancy, such as with the use of low density materials of construction for the housing components and/or by incorporating gas or gas generating materials into the housing, as described for example in U.S. Pat. No. 9,457,176. In general, the system in the dry and drug-loaded state may have a density in the range of about 0.5 g/mL to about 1.5 g/mL, such as between about 0.7 g/mL to about 1.3 g/mL. In some embodiments, the system in the dry and drug-loaded state has a density that is less than 1 g/mL.

In an embodiment, the intravesical drug delivery system is non-bioerodible. In another embodiment, the intravesical drug delivery system can be made to be completely or partially bioerodible so that no explanation, or retrieval, of the system is required following release of the drug formulation. In some embodiments, the system is partially bioerodible so that the system, upon partial erosion, breaks into non-erodible pieces small enough to be excreted from the bladder. For example, the systems described herein may be designed to conform with the characteristics of those described in U.S. Pat. No. 8,690,840.

The drug delivery systems are sterilized before being inserted into a patient. In one embodiment, the system is sterilized using a suitable process such as gamma irradiation or ethylene oxide sterilization, although other sterilization processes may be used.

The systems described herein may include a radio-opaque portion or structure to facilitate detection or viewing (e.g., by X-ray imaging or fluoroscopy) of the system by a medical practitioner as part of the implantation or retrieval procedure. In one embodiment, the housing is constructed of a material that includes a radio-opaque filler material, such as barium sulfate or another radio-opaque material known in the art. Some housings may be made radio-opaque by blending radio-opaque fillers, such as barium sulfate or another suitable material, during the processing of the material from which the housing is formed. The radio-opaque material may be associated with the retention frame in those embodiments that include a retention frame. Ultrasound imaging or fluoroscopy may be used to image the system in vivo.

In some embodiments, the device constituent of the system comprises a drug-impermeable base material and a drug-permeable stripe material, and the base material is a TPU having 20% BaSO₄ filler, such as Lubrizol's Carbothane™ AC-4075A-B20 or Tecothane™ AR-75A-B20. (Lubrizol Life Science (Bethlehem, PA)).

The drug delivery system may further include a retrieval feature, such as a string, a loop, or other structure that facilitates removal of the system from the patient. In one case, the system may be removed from the bladder by engaging the string to pull the system through the urethra. The system may be configured to assume a relatively narrow or linear shape when pulling the system by the retrieval feature into the lumen of a catheter or cystoscope or into the urethra.

### Retention of the System in a Body Cavity

The systems described herein are elastically deformable between a relatively low profile (e.g., straightened or uncoiled) shape suited for insertion through a lumen into the bladder (or other body cavity) of a patient and a relatively expanded retention shape suited to retain the system within the urinary bladder (or other body cavity). In certain embodiments, the drug delivery system may naturally assume the retention shape and may be deformed, either manually or with the aid of an external apparatus, into the relatively straightened shape for insertion into the body. Once deployed the system may spontaneously or naturally return to the initial, retention shape for retention in the body.

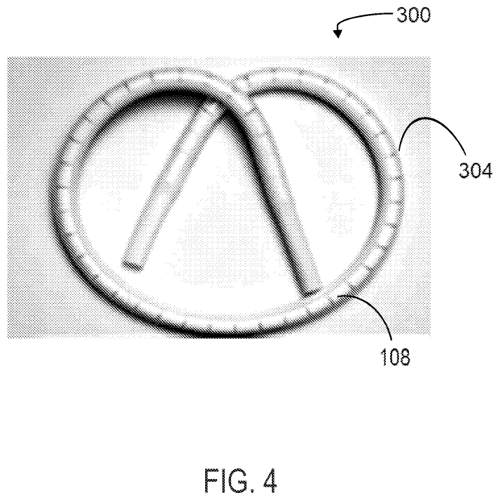

For the purposes of this disclosure, the terms “retention shape,” “relatively expanded shape,” and the like, generally denote any shape suited for retaining the system in the intended implantation location, including, but not limited to, a coiled or “pretzel” shape, such as shown in FIGS. 1 and 4, which is suited for retaining the system in the bladder. Similarly, the terms “deployment shape,” “relatively low profile shape,” “relatively straightened shape,” and the like, generally denote any shape suited for deploying the drug delivery system into the body, including, but not limited to, a linear or elongated shape, such as shown in FIG. 7A-B, which is suited for deploying the system through the working channel of a catheter, cystoscope, or other deployment instrument positioned in a lumen of the body, such as the urethra. For example, the housing or tube of the system may have two opposing free ends, which are directed away from one another when the system is in a low-profile deployment shape and which are directed toward one another when the system is in a relatively expanded retention shape.

In some embodiments, as shown in FIGS. 7A-7C, the system further includes retention frame lumen 734 and a retention frame (not shown) positioned in the retention frame lumen. For example, the retention frame lumen and retention frame may be as described in U.S. Application Publication No. 2010/0331770; U.S. Application Publication No. 2010/0060309; U.S. Application Publication No. 2011/0202036; and U.S. Application Publication No. 2011/0152839, which are incorporated herein by reference. For example, the retention frame lumen may be sealed with a suitable plug or adhesive material, such as a silicone adhesive material.

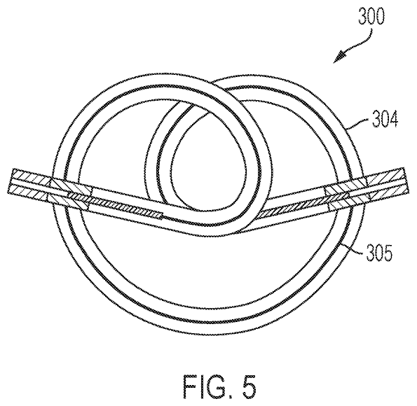

FIG. 4 illustrates a system 300 loaded with drug tablets 108 in the drug reservoir lumen of system housing 304. As can be seen in FIG. 5, prior to loading the tablets, the retention frame 305 urges the system housing 304 into a distinct expanded shape, as compared to the retention shape achieved when the system is loaded with drug tablets 108.

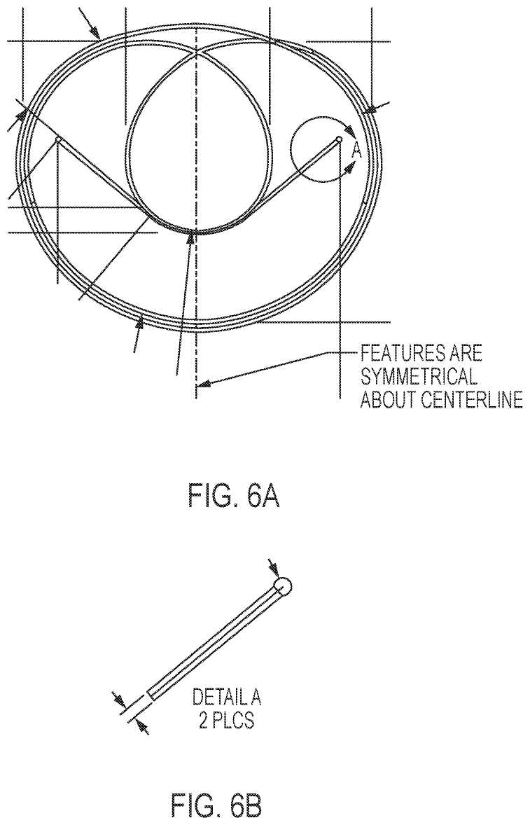

In certain embodiments, in which an increased payload is desired, additional length of the drug reservoir lumen/tube may be provided. In one embodiment, as shown in FIGS. 6A-6B, the retention frame has an outer periphery defined by two overlapping portions (coils) of nitinol wire. Each end portion of the wire is inwardly directed from the periphery and includes (i) a curved transition region having a smaller radius of curvature than the peripheral portions of the wire, and (ii) a straight portion that terminates with a rounded end cap. In contrast, in the system shown in FIG. 5, the retention frame has a periphery defined by a single coil. A system having the retention frame of FIGS. 6A-6B enables a comparatively longer drug reservoir (e.g., to accommodate more tablets) in a system having the same “footprint” (outer peripheral shape and dimension) as the system illustrated in FIG. 5.

In other embodiments, as shown in FIGS. 1-3, the system does not include a retention frame lumen or a retention frame or wire. Instead, the material of the housing is configured to be elastically deformable between the straightened shape and the retention shape, in the absence of a retention frame or wire. In such embodiments, the design and manufacturing of the system is simplified, and the overall size of the system is minimized (or drug payload may be increased where the size of the system remains constant). In embodiments without a retention frame, the tubular housing material serves the functions of (i) forming the drug reservoir lumen, (ii) controlling drug release, and (iii) retaining the system in the bladder upon deployment.

For example, the tubular housing may be thermally shape set to have the retention shape. Thus, the housing may comprise one or more thermoplastic materials that are suitable to be thermally formed into the retention shape. In certain embodiments, a drug delivery system includes a tubular housing having a closed drug reservoir lumen bounded by a wall structure comprising at least one thermoplastic material, wherein (i) at least a portion of the wall structure is water permeable and at least a portion of the wall structure is drug permeable, (ii) the tubular housing is elastically deformable from a retention shape suited to retain the system within the bladder to a relatively straightened shape suited for insertion through a lumen into the bladder, and (iii) the tubular wall is thermally shaped to have the retention shape.

In certain embodiments the first and second wall structures are each a thermoplastic polyurethane and the tubular housing is thermally shaped to have the retention shape. In one embodiment, the tubular wall has a spring constant effective to impede the system from assuming the relatively straightened shape once implanted in the bladder. Thus, the properties of the tubular wall may cause the system to function as a spring, deforming in response to a compressive load but spontaneously returning to its initial shape once the load is removed.

In certain embodiments, the systems may naturally assume the retention shape, may be deformed into the relatively straightened shape, and may spontaneously return to the retention shape upon insertion into the body. The tubular wall structure in the retention shape may be shaped for retention in a body cavity, and in the relatively straightened shape may be shaped for insertion into the body through the working channel of a deployment instrument such as a catheter or cystoscope. To achieve such a result, the tubular wall structure may have an elastic limit, modulus, and/or spring constant selected to impede the system from assuming the relatively lower-profile shape once implanted. Such a configuration may limit or prevent accidental expulsion of the system from the body under expected forces. For example, the system may be retained in the bladder during urination or contraction of the detrusor muscle.

In a preferred embodiment, the system is elastically deformable between a relatively straightened shape suited for insertion through a catheter or cystoscope extending through a patient's urethra of a patient and a curved or coiled shape suited to retain the system within the bladder (i.e., to prevent its expulsion from the bladder during urination) following release of the system from the end of the catheter or cystoscope.

As shown in FIG. 1, the retention shape may include a coiled or “pretzel” shape. The pretzel shape essentially comprises at least two sub-circles, each having its own smaller arch and sharing a common larger arch. When the pretzel shape is first compressed, the larger arch absorbs the majority of the compressive force and begins deforming, but with continued compression the smaller arches overlap, and subsequently, all three of the arches resist the compressive force. The resistance to compression of the system as a whole increases once the two sub-circles overlap, impeding collapse and voiding of the system as the bladder contracts during urination.

The wall structure in the retention shape may have a two-dimensional structure that is confined to a plane, a three-dimensional structure, such as a structure that occupies the interior of a spheroid, or some combination thereof. The retention shape may comprise one or more loops, curls, or sub-circles, connected either linearly or radially, turning in the same or in alternating directions, and overlapping or not overlapping. The retention shape may comprise one or more circles or ovals arranged in a two-dimensional or a three-dimensional configuration, the circles or ovals may be either closed or opened, having the same or different sizes, overlapping or not overlapping, and joined together at one or more connecting points. The retention shape also may be a three-dimensional structure that is shaped to occupy or wind about a spheroid-shaped space, such as a spherical space, a space having a prorate spheroid shape, or a space having an oblate spheroid shape. The wall structure in the retention shape may be shaped to occupy or wind about a spherical space. The wall structure in the retention shape may generally take the shape of two intersecting circles lying in different planes, two intersecting circles lying in different planes with inwardly curled ends, three intersecting circles lying in different planes, or a spherical spiral. In each of these examples, the wall structure can be stretched to the linear shape for deployment through a deployment instrument. The wall structure may wind about or through the spherical space, or other spheroid-shaped space, in a variety of other manners.

Drug delivery systems utilizing thermally formed coextruded tubing with drug permeable and drug impermeable portions may integrate three functional components (drug reservoir/housing, drug permeation route, and retentive feature) into a single thermally shaped co-extruded tubing component, which may simplify the system design and the ability to control the drug release rate. As discussed herein, in such systems, the drug release rate can be relatively easily modified by controlling the angle and thickness of the drug permeable portion (e.g., strip) without changing whole tube housing material.

A thermally shaped coextruded tubular housing may be loaded with drug tablets and both ends may be sealed thermally or with adhesive (such as with the first wall material). If the local tube cross-section deformation or tube kinking occurs, the tablet loading will be difficult. Therefore, the tube dimensions should be chosen to prevent kinking when the tube is thermally shaped. The critical bending radius of curvature (R*) of elastic tubes under pure bending condition can be approximated using the following equation:

$${R}^{*} \approx \frac{3}{\sqrt{2}} \frac{{r}^{2 \sqrt{1 - {v}^{2}}}}{w}$$

where v is Poisson's ratio, r is the mean radius (i.e. (ID+OD)/4), w is the tube wall thickness, ID is tube inner diameter, and OD is tube outer diameter. With a Poisson's ratio v of 0.49 for polyurethanes, the estimated critical radius is 0.5 cm. Therefore, in some embodiments, when thermally shaping a polyuretiane tube, the radius of curvature should preferably be above 0.5 cm all along the length of the tube to prevent kinking. Thus, in one embodiment, the retention shape comprises at least one loop having a radius of curvature of at least 0.5 cm.

### Drug Tablets

As discussed herein with reference to the erdafitinib pharmaceutical formulations, the drug may be provided in a solid form suitable for being loaded within the drug reservoir lumen of the system (e.g., solid mini-tablets). In a preferred embodiment, as shown in FIG. 1, a drug formulation is formed into drug units 108 that are loaded into the drug reservoir lumen of the system 100. Each of the drug units is a solid, discrete object that substantially retains a selectively imparted shape (at the temperature and pressure conditions to which the drug units (e.g., tablets) and the delivery system normally will be exposed during assembly (e.g., loading into the system drug reservoir), storage, and handling before in vivo insertion).

The individual drug units may have essentially any selected shape and dimension that fits within the systems described herein. In one embodiment, the drug units are sized and shaped such that the drug reservoir lumens in the housings are substantially filled by a select number of drug units. Each drug unit may have a cross-sectional shape that substantially corresponds to a cross-sectional shape of the drug reservoir lumen of a particular housing. For example, the drug units may be substantially cylindrical in shape for positioning in a substantially cylindrical drug reservoir lumen. Once loaded, the drug units can, in some embodiments, substantially fill the drug reservoir lumen forming the drug housing portion.

In one embodiment, the drug units are shaped to align in a row when the system is in its deployment configuration. For example, each drug unit may have a cross-sectional shape that corresponds to the cross-sectional shape of the drug reservoir lumens in the housing, and each drug unit may have end face shapes that correspond to the end faces of adjacent drug units. The interstices or breaks between drug units can accommodate deformation or movement of the system, such as during deployment, while permitting the individual drug units to retain their solid form. Thus, the drug delivery system may be relatively flexible or deformable despite being loaded with a solid drug composition, such as a tablet, as each drug unit may be permitted to move with reference to adjacent drug units.

In embodiments in which the drug units are designed for insertion or implantation in a lumen or cavity in the body, such as the bladder, via a drug delivery system, the drug units may be “mini-tablets” that are suitably sized and shaped for insertion through a natural lumen of the body, such as the urethra. For the purpose of this disclosure, the term “mini-tablet” generally indicates a solid drug unit that is substantially cylindrical in shape, having end faces and a side face that is substantially cylindrical. The mini-tablet has a diameter, extending along the end face, in the range of about 1.0 to about 3.2 mm, such as between about 1.5 and about 3.1 mm. The mini-tablet has a length, extending along the side face, in the range of about 1.7 mm to about 4.8 mm, such as between about 2.0 mm and about 4.5 mm. The friability of the tablet may be less than about 2%. In an aspect, the tablets are those as described herein. In an aspect, the tablets are those of Formula 4A. In an aspect, the tablets are those of Formula 4B. In an aspect, the tablets are those of Formula 4C. In an aspect, the tablets are those of Formula 4D.

### Methods of Drug Delivery

The systems and methods or uses disclosed herein may be adapted for use in humans or for use in veterinary or livestock applications. Accordingly, the term “patient” may refer to a human or other mammalian subject. In an embodiment, the patient is a human subject.

In certain embodiments, methods of treatment of urothelial cancers, such as bladder cancers, are provided herein. In certain embodiments, use of a drug delivery system as described herein for the manufacture of a medicament for the treatment of urothelial cancers, such as bladder cancers, are provided herein. In certain embodiments, a drug delivery system as described herein for use in the treatment of urothelial cancers, such as bladder cancers, are provided herein. In certain embodiments, erdafitinib for use in a drug delivery system as described herein for the treatment of urothelial cancers, such as bladder cancers, are provided herein. The methods or uses may include locally delivering or administering erdafitinib (such as in any of the formulations described herein) into the bladder of a patient in need of treatment, in particular a cancer patient, in an amount effective for the treatment of bladder cancer (e.g., from about 1-10 mg/day, as described herein). For example, the treatment may be effective at treating muscle invasive bladder cancer (MIBC), non-muscle invasive bladder cancer (NMIBC), and/or bacillus calmette-guerin (BCG)-naïve bladder cancer. In an aspect the patient, in particular a human, is a BCG-experienced bladder or NMIBC or MIBC cancer patient. In an aspect the patient, in particular a human, is a BCG-naïve bladder or NMIBC or MIBC cancer patient. In an aspect the patient, in particular a human, is a recurrent, bacillus Calmette-Guerin (BCG)-experienced high-risk papillary-only NMIBC (high-grade Ta/T1) cancer patient, refusing or ineligible for radical cystectomy (RCy). In an aspect the patient, in particular a human, is a recurrent, BCG-experienced high-risk papillary-only NMIBC (high-grade Ta/T1) cancer patient, scheduled for RCy. In an aspect the patient, in particular a human, is a recurrent, intermediate-risk NMIBC (Ta and T1) cancer patient with a previous history of only low-grade disease. In an aspect the patient, in particular a human, is a MIBC cancer patient scheduled for RCy who has refused or is ineligible for cisplatin-based neoadjuvant chemotherapy.

In certain embodiments, the urothelial cancers as described herein are susceptible to an FGFR2 genetic alteration and/or an FGFR3 genetic alteration.

As used herein, “FGFR genetic alteration” refers to an alteration in the wild type FGFR gene, including, but not limited to, FGFR fusion genes, FGFR mutations, FGFR amplifications, or any combination thereof, in particular FGFR fusion genes, FGFR mutations, or any combination thereof. In certain embodiments, the FGFR2 or FGFR3 genetic alteration is an FGFR gene fusion. “FGFR fusion” or “FGFR gene fusion” refers to a gene encoding a portion of FGFR (e.g., FGRF2 or FGFR3) and one of the herein disclosed fusion partners, or a portion thereof, created by a translocation between the two genes. The terms “fusion” and “translocation” are used interchangeable herein. The presence of one or more of the following FGFR fusion genes in a biological sample from a patient can be determined using the disclosed methods or uses or by methods known to those of ordinary skill in the art. FGFR3-TACC3, FGFR3-BAIAP2L1, FGFR2-BICC1, FGFR2-CASP7, or any combination thereof. In certain embodiments, FGFR3-TACC3 is FGFR3-TACC3 variant 1 (FGFR3-TACC3 V1) or FGFR3-TACC3 variant 3 (FGFR3-TACC3 V3). Table A provides the FGFR fusion genes and the FGFR and fusion partner exons that are fused. The sequences of the individual FGFR fusion genes are disclosed in Table A2. The underlined sequences correspond to either FGFR3 or FGFR2, the sequences represent the fusion partners.

TABLE A

| Fusion Gene | FGFR Exon | Partner Exon |
|---|---|---|
| FGFR2 | | |
| FGFR2-BICC1 | 19 | 3 |
| FGFR2-CASP7 | 19 | 4 |
| FGFR3 | | |
| FGFR3-BAIAP2L1 | 18 | 2 |
| FGFR3-TACC3 V1 | 18 | 11 |
| FGFR3-TACC3 V3 | 18 | 10 |

TABLE A2

| FGFR3-TACC3 V1 | >ATGGGCGCCCCTGCCTGCGCCCTCGCGCTCTGCGTGGCCGTGGCCATCGTGGCC |
|---|---|
| (2850 base pairs) | GGCGCCTCCTCGGAGTCCTTGGGGACGGAGCAGCGCGTCGTGGGGCGAGCGGCA |
| (SEQ ID NO: 33) | GAAGTCCCGGGCCCAGAGCCCGGCCAGCAGGAGCAGTTGGTCTTCGGCAGCGGG |
| | GATGCTGTGGAGCTGAGCTGTCCCCCGCCCGGGGGTGGTCCCATGGGGCCCACTG |
| | TCTGGGTCAAGGATGGCACAGGGCTGGTGCCCTCGGAGCGTGTCCTGGTGGGGC |
| | CCCAGCGGCTGCAGGTGCTGAATGCCTCCCACGAGGACTCCGGGGCCTACAGCT |
| | GCCGGCAGCGGCTCACGCAGCGCGTACTGTGCCACTTCAGTGTGCGGGTGACAG |
| | ACGCTCCATCCTCGGGAGATGACGAAGACGGGGAGGACGAGGCTGAGGACACA |
| | GGTGTGGACACAGGGGCCCCTTACTGGACACGGCCCGAGCGGATGGACAAGAAG |
| | CTGCTGGCCGTGCCGGCCGCCAACACCGTCCGCTTCCGCTGCCCAGCCGCTGGCA |
| | ACCCCACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATGGAAA |
| | GCGTGGTGCCCTCGGACCGCGGCAACTACACCTGCGTCGTGGAGAACAAGTTTG |
| | GCAGCATCCGGCAGACGTACACGCTGGACGTGCTGGAGCGCTCCCCGCACCGGC |
| | CCATCCTGCAGGCGGGGCTGCCGGCCAACCAGACGGCGGTGCTGGGCAGCGACG |
| | TGGAGTTCCACTGCAAGGTGTACAGTGACGCACAGCCCCACATCCAGTGGCTCA |
| | AGCACGTGGAGGTGAATGGCAGCAAGGTGGGCCCGGACGGCACACCCTACGTTA |
| | CCGTGCTCAAGACGGCGGGCGCTAACACCACCGACAAGGAGCTAGAGGTTCTCT |
| | CCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGA |
| | GGAGCTGGTGGAGGCTGACGAGGCGGGCAGTGTGTATGCAGGCATCCTCAGCTA |
| | CGGGGTGGGCTTCTTCCTGTTCATCCTGGTGGTGGCGGCTGTGACGCTCTGCCGC |
| | CTGCGCAGCCCCCCCAAGAAAGGCCTGGGCTCCCCCACCGTGCACAAGATCTCCC |
| | GCTTCCCGCTCAAGCGACAGGTGTCCCTGGAGTCCAACGCGTCCATGAGCTCCAA |
| | CACACCACTGGTGCGCATCGCAAGGCTGTCCTCAGGGGAGGGCCCCACGCTGGC |
| | CAATGTCTCCGAGCTCGAGCTGCCTGCCGACCCCAAATGGGAGCTGTCTCGGGCC |
| | CGGCTGACCCTGGGCAAGCCCCTTGGGGAGGGCTGCTTCGGCCAGGTGGTCATG |
| | GCGGAGGCCATCGGCATTGACAAGGACCGGGCCGCCAAGCCTGTCACCGTAGCC |
| | GTGAAGATGCTGAAAGACGATGCCACTGACAAGGACCTGTCGGACCTGGTGTCT |
| | GAGATGGAGATGATGAAGATGATCGGGAAACACAAAAACATCATCAACCTGCTG |
| | GGCGCCTGCACGCAGGGCGGGCCCCTGTACGTGCTGGTGGAGTACGCGGCCAAG |
| | GGTAACCTGCGGGAGTTTCTGCGGGCGCGGCGGCCCCGGGCCTGGACTACTCCT |
| | TCGACACCTGCAAGCCGCCCGAGGAGCAGCTCACCTTCAAGGACCTGGTGTCCTG |
| | TGCCTACCAGGTGGCCCGGGGCATGGAGTACTTGGCCTCCCAGAAGTGCATCCAC |
| | AGGGACCTGGCTGCCCGCAATGTGCTGGTGACCGAGGACAACGTGATGAAGATC |
| | GCAGACTTCGGGCTGGCCCGGGACGTGCACAACCTCGACTACTACAAGAAGACG |
| | ACCAACGGCCGGCTGCCCGTGAAGTGGATGGCGCCTGAGGCCTTGTTTGACCGA |
| | GTCTACACTCACCAGAGTGACGTCTGGTCCTTTGGGGTCCTGCTCTGGGAGATCT |
| | TCACGCTGGGGGGCTCCCCGTACCCCGGCATCCCTGTGGAGGAGCTCTTCAAGCT |
| | GCTGAAGGAGGGCCACCGCATGGACAAGCCCGCCAACTGCACACACGACCTGTA |
| | CATGATCATGCGGGAGTGCTGGCATGCCGCGCCCTCCCAGAGGCCCACCTTCAAG |
| | CAGCTGGTGGAGGACCTGGACCGTGTCCTTACCGTGACGTCCACCGACGTAAAG |
| | GCGACACAGGAGGAGAACCGGGAGCTGAGGAGCAGGTGTGAGGAGCTCCACGG |
| | GAAGAACCTGGAACTGGGGAAGATCATGGACAGGTTCGAAGAGGTTGTGTACCA |
| | GGCCATGGAGGAAGTTCAGAAGCAGAAGGAACTTTCCAAAGCTGAAATCCAGAA |
| | AGTTCTAAAAGAAAAAGACCAACTTACCACAGATCTGAACTCCATGGAGAAGTC |
| | CTTCTCCGACCTCTTCAAGCGTTTTGAGAAACAGAAAGAGGTGATCGAGGGCTAC |
| | CGCAAGAACGAAGAGTCACTGAAGAAGTGCGTGGAGGATTACCTGGCAAGGATC |
| | ACCCAGGAGGGCCAGAGGTACCAAGCCCTGAAGGCCCACGCGGAGGAGAAGCT |
| | GCAGCTGGCAAACGAGGAGATCGCCCAGGTCCGGAGCAAGGCCCAGGCGGAAG |
| | CGTTGGCCCTCCAGGCCAGCCTGAGGAAGGAGCAGATGCGCATCCAGTCGCTGG |
| | AGAAGACAGTGGAGCAGAAGACTAAAGAGAACGAGGAGCTGACCAGGATCTGC |
| | GACGACCTCATCTCCAAGATGGAGAAGATCTGA |
| FGFR3-TACC3 V3 | >ATGGGCGCCCCTGCCTGCGCCCTCGCGCTCTGCGTGGCCGTGGCCATCGTGGCC |
| (2955 base pairs) | GGCGCCTCCTCGGAGTCCTTGGGGACGGAGCAGCGCGTCGTGGGGCGAGCGGCA |
| (SEQ ID NO: 34) | GAAGTCCCGGGCCCAGAGCCCGGCCAGCAGGAGCAGTTGGTCTTCGGCAGCGGG |
| | GATGCTGTGGAGCTGAGCTGTCCCCCGCCCGGGGGTGGTCCCATGGGGCCCACTG |
| | TCTGGGTCAAGGATGGCACAGGGCTGGTGCCCTCGGAGCGTGTCCTGGTGGGGC |
| | CCCAGCGGCTGCAGGTGCTGAATGCCTCCCACGAGGACTCCGGGGCCTACAGCT |
| | GCCGGCAGCGGCTCACGCAGCGCGTACTGTGCCACTTCAGTGTGCGGGTGACAG |
| | ACGCTCCATCCTCGGGAGATGACGAAGACGGGGAGGACGAGGCTGAGGACACA |
| | GGTGTGGACACAGGGGCCCCTTACTGGACACGGCCCGAGCGGATGGACAAGAAG |
| | CTGCTGGCCGTGCCGGCCGCCAACACCGTCCGCTTCCGCTGCCCAGCCGCTGGCA |
| | ACCCCACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATGGAAA |
| | GCGTGGTGCCCTCGGACCGCGGCAACTACACCTGCGTCGTGGAGAACAAGTTTG |
| | GCAGCATCCGGCAGACGTACACGCTGGACGTGCTGGAGCGCTCCCCGCACCGGC |
| | CCATCCTGCAGGCGGGGCTGCCGGCCAACCAGACGGCGGTGCTGGGCAGCGACG |
| | TGGAGTTCCACTGCAAGGTGTACAGTGACGCACAGCCCCACATCCAGTGGCTCA |
| | AGCACGTGGAGGTGAATGGCAGCAAGGTGGGCCCGGACGGCACACCCTACGTTA |
| | CCGTGCTCAAGACGGCGGGCGCTAACACCACCGACAAGGAGCTAGAGGTTCTCT |
| | CCTTGCACAACGTCACCTTTGAGGACGCCGGGGAGTACACCTGCCTGGCGGGCA |
| | ATTCTATTGGGTTTTCTCATCACTCTGCGTGGCTGGTGGTGCTGCCAGCCGAGGA |
| | GGAGCTGGTGGAGGCTGACGAGGCGGGCAGTGTGTATGCAGGCATCCTCAGCTA |
| | CGGGGTGGGCTTCTTCCTGTTCATCCTGGTGGTGGCGGCTGTGACGCTCTGCCGC |
| | CTGCGCAGCCCCCCCAAGAAAGGCCTGGGCTCCCCCACCGTGCACAAGATCTCCC |
| | GCTTCCCGCTCAAGCGACAGGTGTCCCTGGAGTCCAACGCGTCCATGAGCTCCAA |
| | CACACCACTGGTGCGCATCGCAAGGCTGTCCTCAGGGGAGGGCCCCACGCTGGC |
| | CAATGTCTCCGAGCTCGAGCTGCCTGCCGACCCCAAATGGGAGCTGTCTCGGGCC |
| | CGGCTGACCCTGGGCAAGCCCCTTGGGGAGGGCTGCTTCGGCCAGGTGGTCATG |
| | GCGGAGGCCATCGGCATTGACAAGGACCGGGCCGCCAAGCCTGTCACCGTAGCC |
| | GTGAAGATGCTGAAAGACGATGCCACTGACAAGGACCTGTCGGACCTGGTGTCT |
| | GAGATGGAGATGATGAAGATGATCGGGAAACACAAAAACATCATCAACCTGCTG |
| | GGCGCCTGCACGCAGGGCGGGCCCCTGTACGTGCTGGTGGAGTACGCGGCCAAG |
| | GGTAACCTGCGGGAGTTTCTGCGGGCGCGGCGGCCCCCGGGCCTGGACTACTCCT |
| | TCGACACCTGCAAGCCGCCCGAGGAGCAGCTCACCTTCAAGGACCTGGTGTCCTG |
| | TGCCTACCAGGTGGCCCGGGGCATGGAGTACTTGGCCTCCCAGAAGTGCATCCAC |
| | AGGGACCTGGCTGCCCGCAATGTGCTGGTGACCGAGGACAACGTGATGAAGATC |
| | GCAGACTTCGGGCTGGCCCGGGACGTGCACAACCTCGACTACTACAAGAAGACG |
| | ACCAACGGCCGGCTGCCCGTGAAGTGGATGGCGCCTGAGGCCTTGTTTGACCGA |
| | GTCTACACTCACCAGAGTGACGTCTGGTCCTTTGGGGTCCTGCTCTGGGAGATCT |
| | TCACGCTGGGGGGCTCCCCGTACCCCGGCATCCCTGTGGAGGAGCTCTTCAAGCT |
| | GCTGAAGGAGGGCCACCGCATGGACAAGCCCGCCAACTGCACACACGACCTGTA |
| | CATGATCATGCGGGAGTGCTGGCATGCCGCGCCCTCCCAGAGGCCCACCTTCAAG |
| | CAGCTGGTGGAGGACCTGGACCGTGTCCTTACCGTGACGTCCACCGACGTGCCAG |
| | GCCCACCCCCAGGTGTTCCCGCGCCTGGGGGCCCACCCCTGTCCACCGGACCTAT |
| | AGTGGACCTGCTCCAGTACAGCCAGAAGGACCTGGATGCAGTGGTAAAGGCGAC |
| | ACAGGAGGAGAACCGGGAGCTGAGGAGCAGGTGTGAGGAGCTCCACGGGAAGA |
| | ACCTGGAACTGGGGAAGATCATGGACAGGTTCGAAGAGGTTGTGTACCAGGCCA |
| | TGGAGGAAGTTCAGAAGCAGAAGGAACTTTCCAAAGCTGAAATCCAGAAAGTTC |
| | TAAAAGAAAAAGACCAACTTACCACAGATCTGAACTCCATGGAGAAGTCCTTCT |
| | CCGACCTCTTCAAGCGTTTTGAGAAACAGAAAGAGGTGATCGAGGGCTACCGCA |
| | AGAACGAAGAGTCACTGAAGAAGTGCGTGGAGGATTACCTGGCAAGGATCACCC |
| | AGGAGGGCCAGAGGTACCAAGCCCTGAAGGCCCACGCGGAGGAGAAGCTGCAG |
| | CTGGCAAACGAGGAGATCGCCCAGGTCCGGAGCAAGGCCCAGGCGGAAGCGTTG |
| | GCCCTCCAGGCCAGCCTGAGGAAGGAGCAGATGCGCATCCAGTCGCTGGAGAAG |
| | ACAGTGGAGCAGAAGACTAAAGAGAACGAGGAGCTGACCAGGATCTGCGACGA |
| | CCTCATCTCCAAGATGGAGAAGATCTGA |
| FGFR3-BAIAP2L1 | >ATGGGCGCCCCTGCCTGCGCCCTCGCGCTCTGCGTGGCCGTGGCCATCGTGGCC |
| (3765 base pairs) | GGCGCCTCCTCGGAGTCCTTGGGGACGGAGCAGCGCGTCGTGGGGCGAGCGGCA |
| (SEQ ID NO: 35) | GAAGTCCCGGGCCCAGAGCCCGGCCAGCAGGAGCAGTTGGTCTTCGGCAGCGGG |
| | GATGCTGTGGAGCTGAGCTGTCCCCCGCCCGGGGGTGGTCCCATGGGGCCCACTG |
| | TCTGGGTCAAGGATGGCACAGGGCTGGTGCCCTCGGAGCGTGTCCTGGTGGGGC |
| | CCCAGCGGCTGCAGGTGCTGAATGCCTCCCACGAGGACTCCGGGGCCTACAGCT |
| | GCCGGCAGCGGCTCACGCAGCGCGTACTGTGCCACTTCAGTGTGCGGGTGACAG |
| | ACGCTCCATCCTCGGGAGATGACGAAGACGGGGAGGACGAGGCTGAGGACACA |
| | GGTGTGGACACAGGGGCCCCTTACTGGACACGGCCCGAGCGGATGGACAAGAAG |
| | CTGCTGGCCGTGCCGGCCGCCAACACCGTCCGCTTCCGCTGCCCAGCCGCTGGCA |
| | ACCCCACTCCCTCCATCTCCTGGCTGAAGAACGGCAGGGAGTTCCGCGGCGAGC |
| | ACCGCATTGGAGGCATCAAGCTGCGGCATCAGCAGTGGAGCCTGGTCATGGAAA |
| | GCGTGGTGCCCTCGGACCGCGGCAACTACACCTGCGTCGTGGAGAACAAGTTTG |
| | GCAGCATCCGGCAGACGTACACGCTGGACGTGCTGGAGCGCTCCCCGCACCGGC |
| | CCATCCTGCAGGCGGGGCTGCCGGCCAACCAGACGGCGGTGCTGGGCAGCGACG |
| | TGGAGTTCCACTGCAAGGTGTACAGTGACGCACAGCCCCACATCCAGTGGCTCA |
| | AGCACGTGGAGGTGAATGGCAGCAAGGTGGGCCCGGACGGCACACCCTACGTTA |
| | CCGTGCTCAAGTCCTGGATCAGTGAGAGTGTGGAGGCCGACGTGCGCCTCCGCCT |
| | GGCCAATGTGTCGGAGCGGGACGGGGGCGAGTACCTCTGTCGAGCCACCAATTT |
| | CATAGGCGTGGCCGAGAAGGCCTTTTGGCTGAGCGTTCACGGGCCCCGAGCAGC |
| | CGAGGAGGAGCTGGTGGAGGCTGACGAGGCGGGCAGTGTGTATGCAGGCATCCT |
| | CAGCTACGGGGTGGGCTTCTTCCTGTTCATCCTGGTGGTGGCGGCTGTGACGCTC |
| | TGCCGCCTGCGCAGCCCCCCCAAGAAAGGCCTGGGCTCCCCCACCGTGCACAAG |
| | ATCTCCCGCTTCCCGCTCAAGCGACAGGTGTCCCTGGAGTCCAACGCGTCCATGA |
| | GCTCCAACACACCACTGGTGCGCATCGCAAGGCTGTCCTCAGGGGAGGGCCCCA |
| | CGCTGGCCAATGTCTCCGAGCTCGAGCTGCCTGCCGACCCCAAATGGGAGCTGTC |
| | TCGGGCCCGGCTGACCCTGGGCAAGCCCCTTGGGGAGGGCTGCTTCGGCCAGGT |
| | GGTCATGGCGGAGGCCATCGGCATTGACAAGGACCGGGCCGCCAAGCCTGTCAC |
| | CGTAGCCGTGAAGATGCTGAAAGACGATGCCACTGACAAGGACCTGTCGGACCT |
| | GGTGTCTGAGATGGAGATGATGAAGATGATCGGGAAACACAAAAACATCATCAA |
| | CCTGCTGGGCGCCTGCACGCAGGGCGGGCCCCTGTACGTGCTGGTGGAGTACGC |
| | GGCCAAGGGTAACCTGCGGGAGTTTCTGCGGGCGCGGCGGCCCCCGGGCCTGGA |
| | CTACTCCTTCGACACCTGCAAGCCGCCCGAGGAGCAGCTCACCTTCAAGGACCTG |
| | GTGTCCTGTGCCTACCAGGTGGCCCGGGGCATGGAGTACTTGGCCTCCCAGAAGT |
| | GCATCCACAGGGACCTGGCTGCCCGCAATGTGCTGGTGACCGAGGACAACGTGA |
| | TGAAGATCGCAGACTTCGGGCTGGCCCGGGACGTGCACAACCTCGACTACTACA |
| | AGAAGACGACCAACGGCCGGCTGCCCGTGAAGTGGATGGCGCCTGAGGCCTTGT |
| | TTGACCGAGTCTACACTCACCAGAGTGACGTCTGGTCCTTTGGGGTCCTGCTCTG |
| | GGAGATCTTCACGCTGGGGGGCTCCCCGTACCCCGGCATCCCTGTGGAGGAGCTC |
| | TTCAAGCTGCTGAAGGAGGGCCACCGCATGGACAAGCCCGCCAACTGCACACAC |
| | GACCTGTACATGATCATGCGGGAGTGCTGGCATGCCGCGCCCTCCCAGAGGCCC |
| | ACCTTCAAGCAGCTGGTGGAGGACCTGGACCGTGTCCTTACCGTGACGTCCACCG |
| | ACAATGTTATGGAACAGTTCAATCCTGGGCTGCGAAATTTAATAAACCTGGGGA |
| | AAAATTATGAGAAAGCTGTAAACGCTATGATCCTGGCAGGAAAAGCCTACTACG |
| | ATGGAGTGGCCAAGATCGGTGAGATTGCCACTGGGTCCCCCGTGTCAACTGAACT |
| | GGGACATGTCCTCATAGAGATTTCAAGTACCCACAAGAAACTCAACGAGAGTCT |
| | TGATGAAAATTTTAAAAAATTCCACAAAGAGATTATCCATGAGCTGGAGAAGAA |
| | GATAGAACTTGACGTGAAATATATGAACGCAACTCTAAAAAGATACCAAACAGA |
| | ACACAAGAATAAATTAGAGTCTTTGGAGAAATCCCAAGCTGAGTTGAAGAAGAT |
| | CAGAAGGAAAAGCCAAGGAAGCCGAAACGCACTCAAATATGAACACAAAGAAA |
| | TTGAGTATGTGGAGACCGTTACTTCTCGTCAGAGTGAAATCCAGAAATTCATTGC |
| | AGATGGTTGCAAAGAGGCTCTGCTTGAAGAGAAGAGGCGCTTCTGCTTTCTGGTT |
| | GATAAGCACTGTGGCTTTGCAAACCACATACATTATTATCACTTACAGTCTGCAG |

| | |
|---|---|
| | AACTACTGAATTCCAAGCTGCCTCGGTGGCAGGAGACCTGTGTTGATGCCATCAA |
| | AGTGCCAGAGAAAATCATGAATATGATCGAAGAAATAAAGACCCCAGCCTCTAC |
| | CCCCGTGTCTGGAACTCCTCAGGCTTCACCCATGATCGAGAGAAGCAATGTGGTT |
| | AGGAAAGATTACGACACCCTTTCTAAATGCTCACCAAAGATGCCCCCCGCTCCTT |
| | CAGGCAGAGCATATACCAGTCCCTTGATCGATATGTTTAATAACCCAGCCACGGC |
| | TGCCCCGAATTCACAAAGGGTAAATAATTCAACAGGTACTTCCGAAGATCCCAGT |
| | TTACAGCGATCAGTTTCGGTTGCAACGGGACTGAACATGATGAAGAAGCAGAAA |
| | GTGAAGACCATCTTCCCGCACACTGCGGGCTCCAACAAGACCTTACTCAGCTTTG |
| | CACAGGGAGATGTCATCACGCTGCTCATCCCCGAGGAGAAGGATGGCTGGCTCT |
| | ATGGAGAACACGACGTGTCCAAGGCGAGGGGTTGGTTCCCGTCGTCGTACACGA |
| | AGTTGCTGGAAGAAAATGAGACAGAAGCAGTGACCGTGCCCACGCCAAGCCCCA |
| | CACCAGTGAGAAGCATCAGCACCGTGAACTTGTCTGAGAATAGCAGTGTTGTCAT |
| | CCCCCCACCCGACTACTTGGAATGCTTGTCCATGGGGGCAGCTGCCGACAGGAG |
| | AGCAGATTCGGCCAGGACGACATCCACCTTTAAGGCCCCAGCGTCCAAGCCCGA |
| | GACCGCGGCTCCTAACGATGCCAACGGGACTGCAAAGCCGCCTTTTCTCAGCGG |
| | AGAAAACCCCTTTGCCACTGTGAAACTCCGCCCGACTGTGACGAATGATCGCTCG |
| | GCACCCATCATTCGATGA |
| FGFR2-BICC1 | >ATGGTCAGCTGGGGTCGTTTCATCTGCCTGGTCGTGGTCACCATGGCAACCTTGT |
| (4989 base pairs) | CCCTGGCCCGGCCCTCCTTCAGTTTAGTTGAGGATACCACATTAGAGCCAGAAGA |
| (SEQ ID NO: 36) | GCCACCAACCAAATACCAAATCTCTCAACCAGAAGTGTACGTGGCTGCGCCAGG |
| | GGAGTCGCTAGAGGTGCGCTGCCTGTTGAAAGATGCCGCCGTGATCAGTTGGACT |
| | AAGGATGGGGTGCACTTGGGGCCCAACAATAGGACAGTGCTTATTGGGGAGTAC |
| | TTGCAGATAAAGGGCGCCACGCCTAGAGACTCCGGCCTCTATGCTTGTACTGCCA |
| | GTAGGACTGTAGACAGTGAAACTTGGTACTTCATGGTGAATGTCACAGATGCCAT |
| | CTCATCCGGAGATGATGAGGATGACACCGATGGTGCGGAAGATTTTGTCAGTGA |
| | GAACAGTAACAACAAGAGAGCACCATACTGGACCAACACAGAAAAGATGGAAA |
| | AGCGGCTCCATGCTGTGCCTGCGGCCAACACTGTCAAGTTTCGCTGCCCAGCCGG |
| | GGGGAACCCAATGCCAACCATGCGGTGGCTGAAAAACGGGAAGGAGTTTAAGCA |
| | GGAGCATCGCATTGGAGGCTACAAGGTACGAAACCAGCACTGGAGCCTCATTAT |
| | GGAAAGTGTGGTCCCATCTGACAAGGGAAATTATACCTGTGTAGTGGAGAATGA |
| | ATACGGGTCCATCAATCACACGTACCACCTGGATGTTGTGGAGCGATCGCCTCAC |
| | CGGCCCATCCTCCAAGCCGGACTGCCGGCAAATGCCTCCACAGTGGTCGGAGGA |
| | GACGTAGAGTTTGTCTGCAAGGTTTACAGTGATGCCCAGCCCCACATCCAGTGGA |
| | TCAAGCACGTGGAAAAGAACGGCAGTAAATACGGGCCCGACGGGCTGCCCTACC |
| | TCAAGGTTCTCAAGGCCGCCGGTGTTAACACCACGGACAAAGAGATTGAGGTTC |
| | TCTATATTCGGAATGTAACTTTTGAGGACGCTGGGGAATATACGTGCTTGGCGGG |
| | TAATTCTATTGGGATATCCTTTCACTCTGCATGGTTGACAGTTCTGCCAGCGCCTG |
| | GAAGAGAAAAGGAGATTACAGCTTCCCCAGACTACCTGGAGATAGCCATTTACT |
| | GCATAGGGGTCTTCTTAATCGCCTGTATGGTGGTAACAGTCATCCTGTGCCGAAT |
| | GAAGAACACGACCAAGAAGCCAGACTTCAGCAGCCAGCCGGCTGTGCACAAGCT |
| | GACCAAACGTATCCCCCTGCGGAGACAGGTAACAGTTTCGGCTGAGTCCAGCTCC |
| | TCCATGAACTCCAACACCCCGCTGGTGAGGATAACAACACGCCTCTCTTCAACGG |
| | CAGACACCCCCATGCTGGCAGGGGTCTCCGAGTATGAACTTCCAGAGGACCCAA |
| | AATGGGAGTTTCCAAGAGATAAGCTGACACTGGGCAAGCCCCTGGGAGAAGGTT |
| | GCTTTGGGCAAGTGGTCATGGCGGAAGCAGTGGGAATTGACAAAGACAAGCCCA |
| | AGGAGGCGGTCACCGTGGCCGTGAAGATGTTGAAAGATGATGCCACAGAGAAAG |
| | ACCTTTCTGATCTGGTGTCAGAGATGGAGATGATGAAGATGATTGGGAAACACA |
| | AGAATATCATAAATCTTCTTGGAGCCTGCACACAGGATGGGCCTCTCTATGTCAT |
| | AGTTGAGTATGCCTCTAAAGGCAACCTCCGAGAATACCTCCGAGCCCGGAGGCC |
| | ACCCGGGATGGAGTACTCCTATGACATTAACCGTGTTCCTGAGGAGCAGATGACC |
| | TTCAAGGACTTGGTGTCATGCACCTACCAGCTGGCCAGAGGCATGGAGTACTTGG |
| | CTTCCCAAAAATGTATTCATCGAGATTTAGCAGCCAGAAATGTTTTGGTAACAGA |
| | AAACAATGTGATGAAAATAGCAGACTTTGGACTCGCCAGAGATATCAACAATAT |
| | AGACTATTACAAAAAGACCACCAATGGGCGGCTTCCAGTCAAGTGGATGGCTCC |
| | AGAAGCCCTGTTTGATAGAGTATACACTCATCAGAGTGATGTCTGGTCCTTCGGG |
| | GTGTTAATGTGGGAGATCTTCACTTTAGGGGGCTCGCCCTACCCAGGGATTCCCG |
| | TGGAGGAACTTTTTAAGCTGCTGAAGGAAGGACACAGAATGGATAAGCCAGCCA |
| | ACTGCACCAACGAACTGTACATGATGATGAGGGACTGTTGGCATGCAGTGCCCTC |
| | CCAGAGACCAACGTTCAAGCAGTTGGTAGAAGACTTGGATCGAATTCTCACTCTC |
| | ACAACCAATGAGATCATGGAGGAAACAAATACGCAGATTGCTTGGCCATCAAAA |
| | CTGAAGATCGGAGCCAAATCCAAGAAAGATCCCCATATTAAGGTTTCTGGAAAG |
| | AAAGAAGATGTTAAAGAAGCCAAGGAAATGATCATGTCTGTCTTAGACACAAAA |
| | AGCAATCGAGTCACACTGAAGATGGATGTTTCACATACAGAACATTCACATGTA |
| | ATCGGCAAAGGTGGCAACAATATTAAAAAAGTGATGGAAGAAACCGGATGCCAT |
| | ATCCACTTTCCAGATTCCAACAGGAATAACCAAGCAGAAAAAAGCAACCAGGTA |
| | TCTATAGCGGGACAACCAGCAGGAGTAGAATCTGCCCGAGTTAGAATTCGGGAG |
| | CTGCTTCCTTTGGTGCTGATGTTTGAGCTACCAATTGCTGGAATTCTTCAACCGGT |
| | TCCTGATCCTAATTCCCCCTCTATTCAGCATATATCACAAACGTACAATATTTCAG |
| | TATCATTTAAACAGCGTTCCCGAATGTATGGTGCTACTGTCATAGTACGAGGGTC |
| | TCAGAATAACACTAGTGCTGTGAAGGAAGGAACTGCCATGCTGTTAGAACATCTT |
| | GCTGGGAGCTTAGCATCAGCTATTCCTGTGAGCACACAACTAGATATTGCAGCTC |
| | AACATCATCTCTTTATGATGGGTCGAAATGGGAGCAACATCAAACATATCATGCA |
| | GAGAACAGGTGCTCAGATCCACTTTCCTGATCCCAGTAATCCACAAAAGAAATCT |
| | ACCGTCTACCTCCAGGGCACCATTGAGTCTGTCTGTCTTGCAAGGCAATATCTCA |
| | TGGGTTGTCTTCCTCTTGTGTTGATGTTTGATATGAAGGAAGAAATTGAAGTAGA |
| | TCCACAATTCATTGCGCAGTTGATGGAACAGCTTGATGTCTTCATCAGTATTAAA |
| | CCAAAGCCCAAACAGCCAAGCAAGTCTGTGATTGTGAAAAGTGTTGAGCGAAAT |
| | GCCTTAAATATGTATGAAGCAAGGAAATGTCTCCTCGGACTTGAAAGCAGTGGG |
| | GTTACCATAGCAACCAGTCCATCCCCAGCATCCTGCCCTGCCGGCCTGGCATGTC |
| | CCAGCCTGGATATCTTAGCTTCAGCAGGCCTTGGACTCACTGGACTAGGTCTTTT |
| | GGGACCCACCACCTTATCTCTGAACACTTCAACAACCCCAAACTCACTCTTGAAT |
| | GCTCTTAATAGCTCAGTCAGTCCTTTGCAAAGTCCAAGTTCTGGTACACCCAGCC |
| | CCACATTATGGGCACCCCCACTTGCTAATACTTCAAGTGCCACAGGTTTTTCTGCT |
| | ATACCACACCTTATGATTCCATCTACTGCCCAAGCCACATTAACTAATATTTTGTT |
| | GTCTGGAGTGCCCACCTATGGGCACACAGCTCCATCTCCCCCTCCTGGCTTGACT |
| | CCTGTTGATGTCCATATCAACAGTATGCAGACCGAAGGCAAAAAAATCTCTGCTG |
| | CTTTAAATGGACATGCACAGTCTCCAGATATAAAATATGGTGCAATATCCACTTC |
| | ATCACTTGGAGAAAAAGTGCTGAGTGCAAATCACGGGGATCCGTCCATCCAGAC |
| | AAGTGGGTCTGAGCAGACATCTCCCAAATCAAGCCCCACTGAAGGTTGTAATGA |
| | TGCTTTTGTTGAAGTAGGCATGCCTCGAAGTCCTTCCCATTCTGGGAATGCTGGT |
| | GACTTGAAACAGATGATGTGTCCCTCCAAGGTTTCCTGTGCCAAAAGGCAGACA |
| | GTGGAACTATTGCAAGGCACGAAAAACTCACACTTACACAGCACTGACAGGTTG |
| | CTCTCAGACCCTGAACTGAGTGCTACCGAAAGCCCTTTGGCTGACAAGAAGGCTC |
| | CAGGGAGTGAGCGCGCTGCAGAGAGGGCAGCAGCTGCCCAGCAAAACTCCGAA |
| | AGGGCCCACCTTGCTCCACGGTCATCATATGTCAACATGCAGGCATTTGACTATG |
| | AACAGAAGAAGCTATTAGCCACCAAAGCTATGTTAAAGAAACCAGTGGTGACGG |
| | AGGTCAGAACGCCCACAAATACCTGGAGTGGCCTGGGTTTTTCTAAATCCATGCC |
| | AGCTGAAACTATCAAGGAGTTGAGAAGGGCCAATCATGTGTCCTATAAGCCCAC |
| | AATGACAACCACTTATGAGGGCTCATCCATGTCCCTTTCACGGTCCAACAGTCGT |
| | GAGCACTTGGGAGGTGGAAGCGAATCTGATAACTGGAGAGACCGAAATGGAATT |
| | GGACCTGGAAGTCATAGTGAATTTGCAGCTTCTATTGGCAGCCCTAAGCGTAAAC |
| | AAAACAAATCAACGGAACACTATCTCAGCAGTAGCAATTACATGGACTGCATTT |
| | CCTCGCTGACAGGAAGCAATGGCTGTAACTTAAATAGCTCTTTCAAAGGTTCTGA |
| | CCTCCCTGAGCTCTTCAGCAAACTGGGCCTGGGCAAATACACAGATGTTTTCCAG |
| | CAACAAGAGATCGATCTTCAGACATTCCTCACTCTCACAGATCAGGATCTGAAGG |
| | AGCTGGGAATAACTACTTTTGGTGCCAGGAGGAAAATGCTGCTTGCAATTTCAGA |
| | ACTAAATAAAAACCGAAGAAAGCTTTTTGAATCGCCAAATGCACGCACCTCTTTC |
| | CTGGAAGGTGGAGCGAGTGGAAGGCTACCCCGTCAGTATCACTCAGACATTGCT |
| | AGTGTCAGTGGCCGCTGGTAG |
| FGFR2-CASP7 | >ATGGTCAGCTGGGGTCGTTTCATCTGCCTGGTCGTGGTCACCATGGCAACCTTGT |
| (3213 base pairs) | CCCTGGCCCGGCCCTCCTTCAGTTTAGTTGAGGATACCACATTAGAGCCAGAAGA |
| (SEQ ID NO: 37) | GCCACCAACCAAATACCAAATCTCTCAACCAGAAGTGTACGTGGCTGCGCCAGG |
| | GGAGTCGCTAGAGGTGCGCTGCCTGTTGAAAGATGCCGCCGTGATCAGTTGGACT |
| | AAGGATGGGGTGCACTTGGGGCCCAACAATAGGACAGTGCTTATTGGGGAGTAC |
| | TTGCAGATAAAGGGCGCCACGCCTAGAGACTCCGGCCTCTATGCTTGTACTGCCA |
| | GTAGGACTGTAGACAGTGAAACTTGGTACTTCATGGTGAATGTCACAGATGCCAT |
| | CTCATCCGGAGATGATGAGGATGACACCGATGGTGCGGAAGATTTTGTCAGTGA |
| | GAACAGTAACAACAAGAGAGCACCATACTGGACCAACACAGAAAAGATGGAAA |
| | AGCGGCTCCATGCTGTGCCTGCGGCCAACACTGTCAAGTTTCGCTGCCCAGCCGG |
| | GGGGAACCCAATGCCAACCATGCGGTGGCTGAAAAACGGGAAGGAGTTTAAGCA |
| | GGAGCATCGCATTGGAGGCTACAAGGTACGAAACCAGCACTGGAGCCTCATTAT |
| | GGAAAGTGTGGTCCCATCTGACAAGGGAAATTATACCTGTGTAGTGGAGAATGA |
| | ATACGGGTCCATCAATCACACGTACCACCTGGATGTTGTGGAGCGATCGCCTCAC |
| | CGGCCCATCCTCCAAGCCGGACTGCCGGCAAATGCCTCCACAGTGGTCGGAGGA |
| | GACGTAGAGTTTGTCTGCAAGGTTTACAGTGATGCCCAGCCCCACATCCAGTGGA |
| | TCAAGCACGTGGAAAAGAACGGCAGTAAATACGGGCCCGACGGGCTGCCCTACC |
| | TCAAGGTTCTCAAGGCCGCCGGTGTTAACACCACGGACAAAGAGATTGAGGTTC |
| | TCTATATTCGGAATGTAACTTTTGAGGACGCTGGGGAATATACGTGCTTGGCGGG |
| | TAATTCTATTGGGATATCCTTTCACTCTGCATGGTTGACAGTTCTGCCAGCGCCTG |
| | GAAGAGAAAAGGAGATTACAGCTTCCCCAGACTACCTGGAGATAGCCATTTACT |
| | GCATAGGGGTCTTCTTAATCGCCTGTATGGTGGTAACAGTCATCCTGTGCCGAAT |
| | GAAGAACACGACCAAGAAGCCAGACTTCAGCAGCCAGCCGGCTGTGCACAAGCT |
| | GACCAAACGTATCCCCCTGCGGAGACAGGTAACAGTTTCGGCTGAGTCCAGCTCC |
| | TCCATGAACTCCAACACCCCGCTGGTGAGGATAACAACACGCCTCTCTTCAACGG |
| | CAGACACCCCCATGCTGGCAGGGGTCTCCGAGTATGAACTTCCAGAGGACCCAA |
| | AATGGGAGTTTCCAAGAGATAAGCTGACACTGGGCAAGCCCCTGGGAGAAGGTT |
| | GCTTTGGGCAAGTGGTCATGGCGGAAGCAGTGGGAATTGACAAAGACAAGCCCA |
| | AGGAGGCGGTCACCGTGGCCGTGAAGATGTTGAAAGATGATGCCACAGAGAAAG |
| | ACCTTTCTGATCTGGTGTCAGAGATGGAGATGATGAAGATGATTGGGAAACACA |
| | AGAATATCATAAATCTTCTTGGAGCCTGCACACAGGATGGGCCTCTCTATGTCAT |
| | AGTTGAGTATGCCTCTAAAGGCAACCTCCGAGAATACCTCCGAGCCCGGAGGCC |
| | ACCCGGGATGGAGTACTCCTATGACATTAACCGTGTTCCTGAGGAGCAGATGACC |
| | TTCAAGGACTTGGTGTCATGCACCTACCAGCTGGCCAGAGGCATGGAGTACTTGG |
| | CTTCCCAAAAATGTATTCATCGAGATTTAGCAGCCAGAAATGTTTTGGTAACAGA |
| | AAACAATGTGATGAAAATAGCAGACTTTGGACTCGCCAGAGATATCAACAATAT |
| | AGACTATTACAAAAAGACCACCAATGGGCGGCTTCCAGTCAAGTGGATGGCTCC |
| | AGAAGCCCTGTTTGATAGAGTATACACTCATCAGAGTGATGTCTGGTCCTTCGGG |
| | GTGTTAATGTGGGAGATCTTCACTTTAGGGGGCTCGCCCTACCCAGGGATTCCCG |
| | TGGAGGAACTTTTTAAGCTGCTGAAGGAAGGACACAGAATGGATAAGCCAGCCA |
| | ACTGCACCAACGAACTGTACATGATGATGAGGGACTGTTGGCATGCAGTGCCCTC |
| | CCAGAGACCAACGTTCAAGCAGTTGGTAGAAGACTTGGATCGAATTCTCACTCTC |
| | ACAACCAATGAGATGGCAGATGATCAGGGCTGTATTGAAGAGCAGGGGGTTGAG |
| | GATTCAGCAAATGAAGATTCAGTGGATGCTAAGCCAGACCGGTCCTCGTTTGTAC |
| | CGTCCCTCTTCAGTAAGAAGAAGAAAAATGTCACCATGCGATCCATCAAGACCA |
| | CCCGGGACCGAGTGCCTACATATCAGTACAACATGAATTTTGAAAAGCTGGGCA |
| | AATGCATCATAATAAACAACAAGAACTTTGATAAAGTGACAGGTATGGGCGTTC |
| | GAAACGGAACAGACAAAGATGCCGAGGCGCTCTTCAAGTGCTTCCGAAGCCTGG |
| | GTTTTGACGTGATTGTCTATAATGACTGCTCTTGTGCCAAGATGCAAGATCTGCTT |
| | AAAAAAGCTTCTGAAGAGGACCATACAAATGCCGCCTGCTTCGCCTGCATCCTCT |
| | TAAGCCATGGAGAAGAAAATGTAATTTATGGGAAAGATGGTGTCACACCAATAA |
| | AGGATTTGACAGCCCACTTTAGGGGGGATAGATGCAAAACCCTTTTAGAGAAAC |
| | CCAAACTCTTCTTCATTCAGGCTTGCCGAGGGACCGAGCTTGATGATGGCATCCA |
| | GGCCGACTCGGGGCCCATCAATGACACAGATGCTAATCCTCGATACAAGATCCC |
| | AGTGGAAGCTGACTTCCTCTTCGCCTATTCCACGGTTCCAGGCTATTACTCGTGG |
| | AGGAGCCCAGGAAGAGGCTCCTGGTTTGTGCAAGCCCTCTGCTCCATCCTGGAGG |
| | AGCACGGAAAAGACCTGGAAATCATGCAGATCCTCACCAGGGTGAATGACAGAG |
| | TTGCCAGGCACTTTGAGTCTCAGTCTGATGACCCACACTTCCATGAGAAGAAGCA |
| | GATCCCCTGTGTGGTCTCCATGCTCACCAAGGAACTCTACTTCAGTCAATAG |

FGFR genetic alterations include FGFR single nucleotide polymorphism (SNP). “FGFR single nucleotide polymorphism” (SNP) refers to a FGFR2 or FGFR3 gene in which a single nucleotide differs among individuals. In certain embodiments, the FGFR2 or FGFR3 genetic alteration is an FGFR3 gene mutation. In particular, FGFR single nucleotide polymorphism” (SNP) refers to a FGFR3 gene in which a single nucleotide differs among individuals. The presence of one or more of the following FGFR SNPs in a biological sample from a patient can be determined by methods known to those of ordinary skill in the art or methods disclosed in WO 2016/048833, FGFR3 R248C, FGFR3 S249C, FGFR3 G370C, FGFR3 Y373C, or any combination thereof. The sequences of the FGFR SNPs are provided in Table B.

TABLE B

| FGFR3 mutant | Sequence |
|---|---|
| FGFR3 R248C | TCGGACCGCGGCAACTACACCTGCGTCGTGGAGAACAAGTTTGGCAGCA |
| | TCCGGCAGACGTACACGCTGGACGTGCTGGAG(T)GCTCCCCGCACCGGC |
| | CCATCCTGCAGGCGGGGCTGCCGGCCAACCAGACGGCGGTGCTGGGCAG |
| | CGACGTGGAGTTCCACTGCAAGGTGTACAGTGACGCACAGCCCCACATC |
| | CAGTGGCTCAAGCACGTGGAGGTGAATGGCAGCAAGGTGGGCCCGGACG |
| | GCACACCCTACGTTACCGTGCTCA |
| | (SEQ ID NO: 1) |
| FGFR3 S249C | GACCGCGGCAACTACACCTGCGTCGTGGAGAACAAGTTTGGCAGCATCC |
| | GGCAGACGTACACGCTGGACGTGCTGGGTGAGGGCCCTGGGGCGGCGCG |
| | GGGGTGGGGGCGGCAGTGGCGGTGGTGGTGAGGGAGGGGGTGGCCCCT |
| | GAGCGTCATCTGCCCCCACAGAGCGCT(G)CCCGCACCGGCCCATCCTGCA |
| | GGCGGGGCTGCCGGCCAACCAGACGGCGGTGCTGGGCAGCGACGTGGAG |
| | TTCCACTGCAAGGTGTACAGTGACGCACAGCCCCACATCCAGTGGCTCAA |
| | GCACGTGGAGGTGAATGGCAGCAAGGTGGGCCCGGACGGCACACCCTAC |
| | GTTACCGTGCTCAAGGTGGGCCACCGTGTGCACGT |
| | (SEQ ID NO: 2) |
| FGFR3 G370C | GCGGGCAATTCTATTGGGTTTTCTCATCACTCTGCGTGGCTGGTGGTGCT |
| | GCCAGCCGAGGAGGAGCTGGTGGAGGCTGACGAGGCG(T)GCAGTGTGTA |
| | TGCAGGCATCCTCAGCTACGGGGTGGGCTTCTTCCTGTTCATCCTGGTGG |
| | TGGCGGCTGTGACGCTCTGCCGCCTGCGCAGCCCCCCCAAGAAAGGCCT |
| | GGGCTCCCCCACCGTGCACAAGATCTCCCGCTTCCCG |
| | (SEQ ID NO: 3) |
| FGFR3 Y373C* | CTAGAGGTTCTCTCCTTGCACAACGTCACCTTTGAGGACGCCGGGGAGTA |
| | CACCTGCCTGGCGGGCAATTCTATTGGGTTTTCTCATCACTCTGCGTGGCT |
| | GGTGGTGCTGCCAGCCGAGGAGGAGCTGGTGGAGGCTGACGAGGCGGGC |
| | AGTGTGT(G)TGCAGGCATCCTCAGCTACGGGGTGGGCTTCTTCCTGTTCA |
| | TCCTGGTGGTGGCGGCTGTGACGCTCTGCCGCCTGCGCAGCCCCCCCAAG |
| | AAAGGCCTGGGCTCCCCCACCGTGCACAAGATCTCCCGCTTCCCGCTCAA |
| | GC |
| | (SEQ ID NO: 4) |

Sequences correspond to nucleotides 920-1510 of FGFR3 (Genebank ID # NM_000142.4).
Nucleotides in bold underline represent the SNP.
*Sometimes mistakenly referred to as Y375C in the literature.

In certain embodiments, the methods of or uses for treating an urothelial carcinoma as described herein comprise, consist of, or consist essentially of administering the drug delivery system as described herein to a patient that has been diagnosed with an urothelial carcinoma as described herein and harbors at least one FGFR2 genetic alteration and/or FGFR3 genetic alteration (i.e., one or more FGFR2 genetic alteration, one or more FGFR3 genetic alteration, or a combination thereof). In certain embodiments, the FGFR2 genetic alteration and/or FGFR3 genetic alteration is an FGFR3 gene mutation, FGFR2 gene fusion, or FGFR3 gene fusion. In some embodiments, the FGFR3 gene mutation is R248C, S249C, G370C, Y373C, or any combination thereof. In still further embodiments, the FGFR2 or FGFR3 gene fusion is FGFR3-TACC3, FGFR3-BAIAP2L1, FGFR2-BICC1, FGFR2-CASP7, or any combination thereof.

Also described herein are methods or uses of treating an urothelial carcinoma as described herein comprising, consisting of, or consisting essential of: (a) evaluating a biological sample from a patient with an urothelial carcinoma as described herein for the presence of one or more FGFR gene alterations, in particular one or more FGFR2 or FGFR3 gene alterations; and (b) administering a drug delivery system as described herein to the patient if one or more FGFR gene alterations, in particular one or more FGFR2 or FGFR3 gene alterations, is present in the sample.

The following methods for evaluating a biological sample for the presence of one or more FGFR genetic alterations apply equally to any of the above disclosed methods of treatment and uses.

Suitable methods for evaluating a biological sample for the presence of one or more FGFR genetic alterations are described herein and in WO 2016/048833 and U.S. patent application Ser. No. 16/723,975, which are incorporated herein in their entireties. For example, and without intent to be limiting, evaluating a biological sample for the presence of one or more FGFR genetic alterations can comprise any combination of the following steps: isolating RNA from the biological sample; synthesizing cDNA from the RNA; and amplifying the cDNA (preamplified or non-preamplified). In some embodiments, evaluating a biological sample for the presence of one or more FGFR genetic alterations can comprise: amplifying cDNA from the patient with a pair of primers that bind to and amplify one or more FGFR genetic alterations; and determining whether the one or more FGFR genetic alterations are present in the sample. In some aspects, the cDNA can be pre-amplified. In some aspects, the evaluating step can comprise isolating RNA from the sample, synthesizing cDNA from the isolated RNA, and pre-amplifying the cDNA.

Suitable primer pairs for performing an amplification step include, but are not limited to, those disclosed in WO 2016/048833, as exemplified below in Table C:

TABLE C

| Target | Forward Primer | Reverse Primer 5′-3′ |
|---|---|---|
| FGFR3-TACC3 | GACCTGGACCGTGTCCTTACC | CTTCCCCAGTTCCAGGTTCTT |
| V1 | (SEQ ID NO: 5) | (SEQ ID NO: 6) |
| FGFR3-TACC3 | AGGACCTGGACCGTGTCCTT | TATAGGTCCGGTGGACAGGG |
| V3 | (SEQ ID NO: 7) | (SEQ ID NO: 8) |
| FGFR3- | CTGGACCGTGTCCTTACCGT | GCAGCCCAGGATTGAACTGT |
| BAIAP2L1 | (SEQ ID NO: 9) | (SEQ ID NO: 10) |
| FGFR2-BICC1 | TGGATCGAATTCTCACTCTCA | GCCAAGCAATCTGCGTATTTG |
| | CA | (SEQ ID NO: 12) |
| | (SEQ ID NO: 11) | |
| FGFR2-CASP7 | GCTCTTCAATACAGCCCTGAT | ACTTGGATCGAATTCTCACTCT |
| | CA | CA |
| | (SEQ ID NO: 13) | (SEQ ID NO: 14) |
| FGFR2-CCDC6 | TGGATCGAATTCTCACTCTCA | GCAAAGCCTGAATTTTCTTGA |
| | CA | ATAA |
| | (SEQ ID NO: 15) | (SEQ ID NO: 16) |
| FGFR3 R248C | GCATCCGGCAGACGTACA | CCCCGCCTGCAGGAT |
| | (SEQ ID NO: 17) | (SEQ ID NO: 18) |
| FGFR3 S249C | GCATCCGGCAGACGTACA | CCCCGCCTGCAGGAT |
| | (SEQ ID NO: 19) | (SEQ ID NO: 20) |
| FGFR3 G370C | AGGAGCTGGTGGAGGCTGA | CCGTAGCTGAGGATGCCTG |
| | (SEQ ID NO: 21) | (SEQ ID NO: 22) |
| FGFR3 Y373C | CTGGTGGAGGCTGACGAG | AGCCCACCCCGTAGCT |
| | (SEQ ID NO: 23) | (SEQ ID NO: 24) |
| FGFR3 R248C | GTCGTGGAGAACAAGTTTGGC | GTCTGGTTGGCCGGCAG |
| | (SEQ ID NO: 25) | (SEQ ID NO: 26) |
| FGFR3 S249C | GTCGTGGAGAACAAGTTTGGC | GTCTGGTTGGCCGGCAG |
| | (SEQ ID NO: 27) | (SEQ ID NO: 28) |
| FGFR3 G370C | AGGAGCTGGTGGAGGCTGA | CCGTAGCTGAGGATGCCTG |
| | (SEQ ID NO: 29) | (SEQ ID NO: 30) |
| FGFR3 Y373C | GACGAGGCGGGCAGTG | GAAGAAGCCCACCCCGTAG |
| | (SEQ ID NO: 31) | (SEQ ID NO: 32) |

The presence of one or more FGFR genetic alterations can be evaluated at any suitable time point including upon diagnosis, following tumor resection, following first-line therapy, during clinical treatment, or any combination thereof.

The methods and uses can further comprise evaluating the presence of one or more FGFR genetic alterations in the biological sample before the administering step.

The diagnostic tests and screens are typically conducted on a biological sample selected from blood, lymph fluid, bone marrow, a solid tumor sample, or any combination thereof. In certain embodiments, the biological sample is a solid tumor sample. In certain embodiments, the biological sample is a blood sample, or a urine sample.

Methods of identification and analysis of genetic alterations and up-regulation of proteins are known to a person skilled in the art. Screening methods could include, but are not limited to, standard methods such as reverse-transcriptase polymerase chain reaction (RT PCR) or in-situ hybridization such as fluorescence in situ hybridization (FISH).

Identification of an individual carrying a genetic alteration in FGFR, in particular an FGFR genetic alteration as described herein, may mean that the patient would be particularly suitable for treatment with erdafitinib. Tumors may preferentially be screened for presence of a FGFR variant prior to treatment. The screening process will typically involve direct sequencing, oligonucleotide microarray analysis, or a mutant specific antibody. In addition, diagnosis of tumor with such genetic alteration could be performed using techniques known to a person skilled in the art and as described herein such as RT-PCR and FISH.

In addition, genetic alterations of, for example FGFR, can be identified by direct sequencing of, for example, tumor biopsies using PCR and methods to sequence PCR products directly as hereinbefore described. The skilled artisan will recognize that all such well-known techniques for detection of the over expression, activation or mutations of the aforementioned proteins could be applicable in the present case.

In screening by RT-PCR, the level of mRNA in the tumor is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. (2004) Current Protocols in Molecular Biology, John Wiley & Sons Inc., or Innis, M. A. et al., eds. (1990) PCR Protocols: a guide to methods and applications, Academic Press, San Diego. Reactions and manipulations involving nucleic acid techniques are also described in Sambrook et al., (2001), 3rd Ed, Molecular Cloning: A Laboratory Manual, Cold Spring Harbor Laboratory Press. Alternatively, a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference. An example of an in-situ hybridization technique for assessing mRNA expression would be fluorescence in-situ hybridization (FISH) (see Angerer (1987) Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. (2004) Current Protocols in Molecular Biology, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Methods for gene expression profiling are described by (DePrimo et al. (2003), BMC Cancer, 3:3). Briefly, the protocol is as follows: double-stranded cDNA is synthesized from total RNA Using a (dT)24 oligomer (SEQ ID NO: 38: tttttttttt tttttttttt tttt) for priming first-strand cDNA synthesis, followed by second strand cDNA synthesis with random hexamer primers. The double-stranded cDNA is used as a template for in vitro transcription of cRNA using biotinylated ribonucleotides. cRNA is chemically fragmented according to protocols described by Affymetrix (Santa Clara, CA, USA), and then hybridized overnight on Human Genome Arrays.

Alternatively, the protein products expressed from the mRNAs may be assayed by immunohistochemistry of tumor samples, solid phase immunoassay with microtitre plates, Western blotting, 2-dimensional SDS-polyacrylamide gel electrophoresis, ELISA, flow cytometry and other methods known in the art for detection of specific proteins. Detection methods would include the use of site-specific antibodies. The skilled person will recognize that all such well-known techniques for detection of upregulation of FGFR or detection of FGFR variants or mutants could be applicable in the present case.

Abnormal levels of proteins such as FGFR can be measured using standard enzyme assays, for example, those assays described herein. Activation or overexpression could also be detected in a tissue sample, for example, a tumor tissue, by measuring the tyrosine kinase activity with an assay such as that from Chemicon International. The tyrosine kinase of interest would be immunoprecipitated from the sample lysate and its activity measured.

Alternative methods for the measurement of the over expression or activation of FGFR including the isoforms thereof, include the measurement of microvessel density. This can for example be measured using methods described by Orre and Rogers (Int J Cancer (1999), 84(2) 101-8). Assay methods also include the use of markers.

Therefore, all of these techniques could also be used to identify tumors particularly suitable for treatment with the drug delivery systems of the invention.

According to certain embodiments, FGFR2 and/or FGFR3 genetic alterations can be identified using commercially available kits including, but not limiting to, a QIAGEN Therascreen® FGFR RGQ RT-PCR kit.

In certain embodiments, a method of administering a drug to a patient includes inserting a drug delivery system as described herein into a patient and permitting the drug to be released from the system. For example, the system may include any features, or combinations of features, described herein. In one embodiment, the drug is released from the drug reservoir lumen via diffusion through the second material of the wall structure. In certain embodiments, a release profile of the drug is substantially independent of pH over a pH range of 5 to 7. In certain embodiments, a release profile of the drug is substantially independent of pH over a pH range of 5.5 to 7. In certain embodiments, a release profile of the drug is substantially independent of pH over a pH range of 5.5 to 8.

In certain embodiments, permitting the drug to be released from the system includes permitting water to be imbibed through the water permeable wall portions (e.g., through only the second wall structure/second material or through both the first and second wall structures/materials to solubilize the drug), and permitting the solubilized drug to be released from the system by diffusion through the second wall structure/material. That is, in certain embodiments, elution of drug from the system occurs following dissolution of the drug within the system. Bodily fluid enters the system, contacts the drug and solubilizes the drug, and thereafter the dissolved drug diffuses from the system. For example, the drug may be solubilized upon contact with urine in cases in which the system is inserted into the bladder. In one embodiment, releasing the drug from the system includes solubilizing the drug with water or an aqueous medium, such as for example urine, imbibed through the second wall structure/material, or both the first and second wall structures/materials.

In some embodiments, the device constituent of the system comprises a water-permeable and drug-impermeable base material and a water- and drug-permeable stripe material. For example, the base material may be a TPU such as Lubrizol's Carbothane™ AC-4075A or Tecothane™ AR-75A, and the stripe material may be a TPU such as a Lubrizol TECOFLEX™ TPU, such as EG-80A. (Lubrizol Life Science (Bethlehem, PA)).

In certain embodiments, the inserting comprises deploying the system through the patient's urethra and into the patient's urinary bladder. The system may release drug for several days, weeks, months, or more after the implantation procedure has ended. In one embodiment, deploying the drug delivery system in the patient includes inserting the system into a body cavity or lumen of the patient via a deployment instrument. For example, the system may be deployed through a deployment instrument, such as a catheter or cystoscope, positioned in a natural lumen of the body, such as the urethra, or into a body cavity, such as the bladder. The deployment instrument typically is removed from the body lumen while the drug delivery system remains in the bladder or other body cavity for a prescribed treatment period.

In one example, the system is deployed by passing the drug delivery system through a deployment instrument and releasing the system from the deployment instrument into the body of the patient, e.g., in a body cavity such as the bladder. In embodiments, the system assumes a retention shape, such as an expanded or higher profile shape, once the system emerges from the deployment instrument into the cavity. The deployment instrument may be a commercially available system or a system specially adapted for the present drug delivery systems. In one embodiment, deploying the drug delivery system in the patient includes (i) elastically deforming the system into the relatively straightened shape; (ii) inserting the system through the patient's urethra; and (iii) releasing the system into the patient's bladder such that it assumes a coiled retention shape.

The drug delivery system may be passed through the deployment instrument, for example driven by a stylet, typically with aid of a lubricant, until the drug delivery system exits a lumen of the instrument and passes into the bladder.

In particular embodiments, the drug delivery systems described herein are deployed into a patient's bladder transurethrally using a Urinary Placement Catheter, which comprises two components: a catheter-like shaft and a stylet that fits inside the shaft. The shaft may include a single lumen extrusion with an atraumatic distal tip that includes a Coude bend, an exit port near the distal tip, and an internal lumen that extends from the exit port to an open proximal end. Depth markings on the shaft indicate insertion depth and orientation of the Coudé tip to assist with the intravesical drug delivery system insertion procedure. The stylet is a single lumen extrusion and is used to advance the drug delivery system through the clear shaft lumen and into the bladder.

Once deployed in vivo, the system subsequently releases the drug (e.g., erdafitinib) for the treatment of one or more conditions or diseases, locally to tissues at the deployment site. The release is controlled to release the drug in an effective amount over an extended period. Thereafter, the system may be removed, resorbed, excreted, or some combination thereof. In certain embodiments, the system resides in the bladder releasing the drug over a predetermined period, such as two weeks, three weeks, four weeks, a month, two months, three months or more.

The deployed system releases a desired quantity of drug over a desired, predetermined period. In embodiments, the system can deliver the desired dose of drug over an extended period, such as 12 hours, 24 hours, 2 days, 3 days, 5 days, 7 days, 10 days, 14 days, or 20, 25, 30, 45, 60, or 90 days, 6 months, or more. The rate of delivery and dosage of the drug can be selected depending upon the drug being delivered and the disease or condition being treated. In one embodiment, a rate of release of the drug from the drug delivery system is zero order over at least 36 hours. In one embodiment, a rate of the release of the drug from the drug delivery system is essentially zero order over at least 7 days, two weeks, three weeks, four weeks, a month, two months, three months or more.

Subsequently, the system may be retrieved from the body, such as in cases in which the system is non-bioerodible or otherwise needs to be removed. Retrieval systems for this purpose are known in the art or can be specially produced. The system also may be completely or partially bioerodible, resorbable, or biodegradable, such that retrieval is unnecessary, as either the entire system is resorbed or the system sufficiently degrades for expulsion, for example, from the bladder during urination. The system may not be retrieved or resorbed until some of the drug, or preferably most or all of the drug, has been released. If needed, a new drug-loaded system may subsequently be implanted, during the same procedure as the retrieval or at a later time.

### Methods of Making the Drug Delivery System

The systems described herein generally are formed by using a co-extrusion or 3D-printing process to form the elongated, elastic housing of the system; loading the drug reservoir lumen with a suitable quantity of the drug (e.g., with a suitable number of drug tablets); and closing off the ends of the tubular housing.

In some embodiments, the tubular wall structure may include a retention lumen extending through or along the structure. The retention lumen optionally may be loaded with an elastic retention frame, such as a nitinol wire or other superelastic wire, and then sealed to keep the frame inside the lumen and/or optionally may be filled with a gas (e.g., air) and then sealed at its ends prior or subsequent to drug loading of the system. In another embodiment, the retention lumen may be filled with high durometer silicone, prior to drug loading of the system, which is then cured into a solid, elastic form effective to bias the tubular wall structure in the coiled bladder retention shape.

In other embodiments, the method includes thermally shape setting the tubular structure to have a coiled retention shape which is elastically deformable into an uncoiled shape. In such embodiments, a retention lumen and frame may not be necessary.

Some steps or sub-steps of the method of making a drug delivery system may be performed in other orders or simultaneously.

The present disclosure may be further understood with reference to the following non-limiting examples.

### EMBODIMENTS

1. A solid pharmaceutical composition comprising:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of at least 45 wt % of the solid pharmaceutical composition; and
(b) at least one pharmaceutical excipient.

2. The solid pharmaceutical composition of embodiment 1, wherein the at least one pharmaceutical excipient comprises or is selected from a solubilizer, a binder, a diluent (filler), a wetting agent, a disintegrant, a glidant, a lubricant, a formaldehyde scavenger, or any combination thereof.

3. The solid pharmaceutical composition of embodiment 1, wherein the at least one pharmaceutical excipient comprises or is selected from a solubilizer, a binder, a diluent (filler), a glidant, a lubricant, a formaldehyde scavenger, or any combination thereof.

4. A process for making a solid pharmaceutical composition comprising:
(a) preparing an intragranular solid composition comprising or consisting essentially of:
   (i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine); and
   (ii) at least one intragranular pharmaceutical excipient;
(b) combining the intragranular solid composition with at least one extragranular pharmaceutical excipient to form a blend; and
(c) tableting the blend to form the solid pharmaceutical composition, wherein the erdafitinib free base is present in a concentration of at least 45 wt % of the solid pharmaceutical composition.

5. The process for making a solid pharmaceutical composition according to embodiment 4, wherein at least one intragranular pharmaceutical excipient and at least one extragranular pharmaceutical excipient comprise or are selected from at least one common (mutually occurring) pharmaceutical excipient.

6. The process for making a solid pharmaceutical composition according to embodiment 4, wherein the at least one intragranular excipient and the at least one extragranular pharmaceutical excipient do not comprise a common (mutually occurring) pharmaceutical excipient.

7. The process for making a solid pharmaceutical composition according to any of embodiments 4-6, wherein the intragranular solid composition is prepared by a roller compaction process.

8. The process for making a solid pharmaceutical composition according to any of embodiments 4-6, wherein the intragranular solid composition is prepared by a fluid bed granulation process.

9. The process for making a solid pharmaceutical composition according to any of embodiments 4-8, wherein the at least one extragranular pharmaceutical excipient comprises microcrystalline cellulose and vinylpyrrolidone-vinyl acetate copolymer, in particular in a weight ratio of 50:50.

10. The process for making a solid pharmaceutical composition according to any of embodiments 4-6, wherein:
(a) the intragranular solid composition comprises a solubilizer, at least one binder, and a first quantity of a lubricant;
(b) the extragranular pharmaceutical excipients comprise a diluent, a glidant, and a second quantity of a lubricant; and
(c) the intragranular solid composition is prepared by a roller compaction process.

11. The process for making a solid pharmaceutical composition according to embodiment 10, wherein:
the solubilizer is hydroxypropyl-beta-cyclodextrin;
the binder is a combination of microcrystalline cellulose and vinylpyrrolidone-vinyl acetate copolymer;
the lubricant is magnesium stearate;
the diluent is anhydrous dibasic calcium phosphate; and
the glidant is colloidal silicon dioxide.

12. The process for making a solid pharmaceutical composition according to any of embodiments 4-5, wherein:
(a) the intragranular solid composition comprises a solubilizer, a diluent, and a disintegrant;
(b) the extragranular pharmaceutical excipients comprise at least one binder and a lubricant; and
(c) the intragranular solid composition is prepared by a fluid bed granulation process.

13. The process for making a solid pharmaceutical composition according to embodiment 12, wherein:
the solubilizer comprises or is selected from hydroxypropyl-beta-cyclodextrin;
the diluent comprises or is selected from microcrystalline cellulose;
the disintegrant comprises or is selected from hydroxypropyl methylcellulose;
the at least one binder comprises or is selected from a combination of microcrystalline cellulose and vinylpyrrolidone-vinyl acetate copolymer; and
the lubricant comprises or is selected from magnesium stearate.

14. The solid pharmaceutical composition or the process for making a solid pharmaceutical composition according to any of embodiments 1-13, wherein the erdafitinib free base is present in the solid pharmaceutical composition in a concentration of from 45 wt % to 55 wt %, from 47 wt % to 53 wt %, or about 50 wt %.

15. The solid pharmaceutical composition or the process for making a solid pharmaceutical composition according to any of embodiments 1-13, wherein the erdafitinib free base is present in the solid pharmaceutical composition in a concentration of from 45 wt % to 55 wt %, from 47 wt % to 53 wt %, or about 50 wt % and wherein the at least one extragranular excipient comprises microcrystalline cellulose and vinylpyrrolidone-vinyl acetate copolymer, in particular in a weight ratio of 50:50.

16. The solid pharmaceutical composition or the process for making a solid pharmaceutical composition according to any of embodiments 1-15, wherein the solid pharmaceutical composition further comprises a formaldehyde scavenger.

17. The solid pharmaceutical composition or the process for making a solid pharmaceutical composition according to embodiment 16, wherein the formaldehyde scavenger comprises or is selected from an amino acid, an amino sugar, an alpha-(α-)amine compound, conjugates thereof, or any combination thereof.

18. The solid pharmaceutical composition or the process for making a solid pharmaceutical composition according to embodiment 16, wherein the formaldehyde scavenger comprises or is selected from meglumine, glycine, alanine, serine, threonine, cysteine, valine, leucine, isoleucine, methionine, phenylalanine, tyrosine, aspartic acid, glutamic acid, arginine, lysine, ornithine, taurine, histidine, aspartame, proline, tryptophan, citrulline, pyrrolysine, asparagine, glutamine, tris(hydroxymethyl)aminomethane, conjugates thereof, pharmaceutically acceptable salts thereof, or any combination thereof.

19. The solid pharmaceutical composition or the process for making a solid pharmaceutical composition according to embodiment 16, wherein the formaldehyde scavenger is meglumine.

20. The solid pharmaceutical composition or the process for making a solid pharmaceutical composition according to any of embodiments 16-19, wherein the formaldehyde scavenger is present in the solid pharmaceutical composition in a concentration of from 0.01 wt % to 5 wt %, from 0.05 wt % to 3 wt %, from 0.1 wt % to 2 wt %, from 0.5 wt % to 1.5 wt %, or about 1 wt %.

21. The solid pharmaceutical composition or the process for making a solid pharmaceutical composition according to any of embodiments 1-20, wherein the solid pharmaceutical composition further comprises a compound having the formula

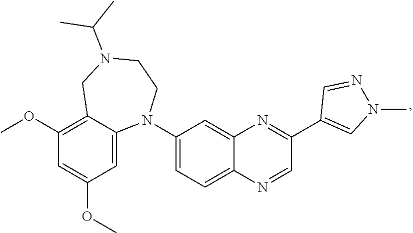

a salt thereof, a solvate thereof, or a combination thereof.

22. The solid pharmaceutical composition or the process for making a solid pharmaceutical composition according to any of embodiments 1-21, wherein the at least one pharmaceutical excipient, the at least one intragranular pharmaceutical excipient or the at least one extragranular pharmaceutical excipient comprises a solubilizer.

23. The solid pharmaceutical composition or the process for making a solid pharmaceutical composition according to embodiment 22, wherein the solubilizer comprises or is selected from (a) a cyclic oligosaccharide, (b) a cellulose which is functionalized with methoxy-, 2-hydroxypropoxy-, acetyl-, or succinoyl-moieties or a combination thereof, or (c) a salt thereof.

24. The solid pharmaceutical composition or the process for making a solid pharmaceutical composition according to embodiment 22, wherein the solubilizer comprises or is selected from hydroxypropyl-beta-cyclodextrin, hydroxypropyl-gamma-cyclodextrin, sulfobutyl ether-beta-cyclodextrin sodium salt, hydroxypropyl methylcellulose acetate succinate, hydroxypropyl methylcellulose E5 (HPMC-E5), or any combination thereof.

25. The solid pharmaceutical composition or the process for making a solid pharmaceutical composition according to embodiment 22, wherein the at least one pharmaceutical excipient or the at least one intragranular pharmaceutical excipient comprises a solubilizer comprising hydroxypropyl-beta-cyclodextrin.

26. The solid pharmaceutical composition or the process for making a solid pharmaceutical composition according to any of embodiments 22-25, wherein the total concentration of the solubilizer in the solid pharmaceutical composition is from 1 wt % to 20 wt %, from 5 wt % to 15 wt %, from 7 wt % to 12 wt %, or about 10 wt %.

27. The solid pharmaceutical composition or the process for making a solid pharmaceutical composition according to any of embodiments 1-26, wherein the at least one pharmaceutical excipient, the at least one intragranular pharmaceutical excipient or the at least one extragranular pharmaceutical excipient comprises or further comprises at least one binder.

28. The solid pharmaceutical composition or the process for making a solid pharmaceutical composition according to embodiment 27, wherein the at least one binder comprises or is selected independently from a water soluble polymeric binder, a slightly water soluble polymeric binder, a water insoluble polymeric binder, or any combination thereof.

29. The solid pharmaceutical composition or the process for making a solid pharmaceutical composition according to embodiment 27, wherein the at least one binder comprises or is selected independently from polyvinylpyrrolidone (PVP), poly(vinyl acetate) (PVA), vinylpyrrolidone-vinyl acetate copolymer, polyethylene oxide (PEO), polypropylene oxide (PPO), an ethylene glycol-propylene glycol copolymer, a poloxamer, hydroxypropyl cellulose (HPC), hydroxypropyl methylcellulose (HPMC), microcrystalline cellulose, silicified microcrystalline cellulose, or combinations thereof.

30. The solid pharmaceutical composition or the process for making a solid pharmaceutical composition according to embodiment 27, wherein the at least one binder comprises or is selected from vinylpyrrolidone-vinyl acetate copolymer, silicified microcrystalline cellulose, microcrystalline cellulose, hydroxypropyl methylcellulose (HPMC), or any combination thereof.

31. The solid pharmaceutical composition or the process for making a solid pharmaceutical composition according to embodiment 27, wherein the at least one binder comprises or is microcrystalline cellulose.

32. The solid pharmaceutical composition or the process for making a solid pharmaceutical composition according to any of embodiments 29-30, wherein the vinylpyrrolidone-vinyl acetate copolymer has a molecular weight (Mw) range of from 45,000 g/mol to 70,000 g/mol.

33. The solid pharmaceutical composition or the process for making a solid pharmaceutical composition according to any of embodiments 27-32, wherein the total concentration of the at least one binder in the solid pharmaceutical composition is from 5 wt % to 30 wt %, from 10 wt % to 25 wt %, from 12 wt % to 22 wt %, or from 14 wt % to 19 wt %.

34. The solid pharmaceutical composition or the process for making a solid pharmaceutical composition according to any of embodiments 27-33, wherein the at least one binder comprises or further comprises a vinylpyrrolidone-vinyl acetate copolymer which is present in the solid pharmaceutical composition in a concentration of from 4 wt % to 12 wt %, from 6 wt % to 10 wt %, or from 7 wt % to 8 wt %.

35. The solid pharmaceutical composition or the process for making a solid pharmaceutical composition according to any of embodiments 27-34, wherein the at least one binder comprises or further comprises: (a) microcrystalline cellulose which is present in the solid pharmaceutical composition in a concentration of from 5 wt % to 20 wt %, from 6 wt % to 15 wt %, or from 7 wt % to 12 wt %; (b) silicified microcrystalline cellulose which is present in the solid pharmaceutical composition in a concentration of from 3 wt % to 18 wt %, from 4 wt % to 15 wt %, or from 5 wt % to 12 wt %; or (c) a combination of both (a) and (b).

36. The solid pharmaceutical composition or the process for making a solid pharmaceutical composition according to any of embodiments 1-35, wherein the at least one pharmaceutical excipient, the at least one intragranular pharmaceutical excipient or the at least one extragranular pharmaceutical excipient comprises or further comprises a wetting agent.

37. The solid pharmaceutical composition or the process for making a solid pharmaceutical composition according to embodiment 36, wherein the wetting agent comprises or is an anionic surfactant.

38. The solid pharmaceutical composition or the process for making a solid pharmaceutical composition according to embodiment 36, wherein the wetting agent comprises or is selected independently from sodium lauryl sulfate, sodium stearyl fumarate, polysorbate 80, docusate sodium, or any combination thereof.

39. The solid pharmaceutical composition or the process for making a solid pharmaceutical composition according to any of embodiments 36-38, wherein the total concentration of the wetting agent in the solid pharmaceutical composition is from 0.01 wt % to 2.5 wt %, from 0.05 wt % to 1.0 wt %, or from 0.1 wt % to 0.5 wt %.

40. The solid pharmaceutical composition or the process for making a solid pharmaceutical composition according to any of embodiments 1-39, wherein the at least one pharmaceutical excipient, the at least one intragranular pharmaceutical excipient or the at least one extragranular pharmaceutical excipient comprises or further comprises a disintegrant.

41. The solid pharmaceutical composition or the process for making a solid pharmaceutical composition according to embodiment 40, wherein the disintegrant comprises or is selected independently from a functionalized polysaccharide or a crosslinked polymer.

42. The solid pharmaceutical composition or the process for making a solid pharmaceutical composition according to embodiment 40, wherein the disintegrant comprises or is selected from (a) a cellulose which is functionalized with methoxy-, 2-hydroxypropoxy-, or carboxymethoxy-moieties, a salt thereof, or a combination thereof, (b) a carboxymethylated starch, or (c) a crosslinked polymer.

43. The solid pharmaceutical composition or the process for making a solid pharmaceutical composition according to embodiment 40, wherein the disintegrant comprises or is selected independently from hydroxypropyl methylcellulose, low-substituted hydroxypropylcellulose, crospovidone (crosslinked polyvinylpyrrolidone), croscarmellose sodium (cross-linked sodium carboxymethylcellulose), sodium starch glycolate, or any combination thereof.

44. The solid pharmaceutical composition or the process for making a solid pharmaceutical composition according to any of embodiments 40-43, wherein the total concentration of the disintegrant in the solid pharmaceutical composition is from 0.1 wt % to 3 wt %, from 0.5 wt % to 2.5 wt %, from 1 wt % to 2 wt %, or about 1.5 wt %.

45. The solid pharmaceutical composition or the process for making a solid pharmaceutical composition according to any of embodiments 1-44, wherein the at least one pharmaceutical excipient, the at least one intragranular pharmaceutical excipient or the at least one extragranular pharmaceutical excipient comprises or further comprises a diluent.

46. The solid pharmaceutical composition or the process for making a solid pharmaceutical composition according to embodiment 45, wherein the diluent comprises or is selected from a sugar, starch, microcrystalline cellulose, a sugar alcohol, a hydrogen phosphate salt, a dihydrogen phosphate salt, a carbonate salt, or combinations thereof.

47. The solid pharmaceutical composition or the process for making a solid pharmaceutical composition according to embodiment 45, wherein the diluent comprises or is selected from lactose (lactose monohydrate), dextrin, mannitol, sorbitol, starch, microcrystalline cellulose, dibasic calciumn phosphate, anhydrous dibasic calciurn phosphate, calcium carbonate, sucrose, or any combination thereof.

48. The solid pharmaceutical composition or the process for making a solid pharmaceutical composition according to any of embodiments 45-47, wherein the total concentration of the diluent in the solid pharmaceutical composition is from 12 wt % to 30 wt %, from 15 wt % to 25 wt %, or from 18 wt % to 22 wt %.

49. The solid pharmaceutical composition or the process for making a solid pharmaceutical composition according to embodiment 47, wherein the diluent comprises or is selected from anhydrous dibasic calcium phosphate in a concentration of from 18 wt % to 20 wt %.

50. The solid pharmaceutical composition or the process for making a solid pharmaceutical composition according to embodiment 47, wherein the diluent comprises or is selected from microcrystalline cellulose in a concentration of from 20 wt % to 22 wt %.

51. The solid pharmaceutical composition or the process for making a solid pharmaceutical composition according to any of embodiments 1-50, wherein the at least one pharmaceutical excipient, the at least one intragranular pharmaceutical excipient or the at least one extragranular pharmaceutical excipient comprises or further comprises a glidant.

52. The solid pharmaceutical composition or the process for making a solid pharmaceutical composition according to embodiment 51, wherein the glidant comprises or is selected from colloidal silicon dioxide, colloidal anhydrous silicon dioxide, talc, or any combination thereof.

53. The solid pharmaceutical composition or the process for making a solid pharmaceutical composition according to embodiment 51, wherein the glidant comprises or is colloidal silicon dioxide.

54. The solid pharmaceutical composition or the process for making a solid pharmaceutical composition according to any of embodiments 51-53, wherein the total concentration of the glidant in the solid pharmaceutical composition is from 0.01 wt % to 5 wt %, 0.05 wt % to 3 wt %, 0.1 wt % to 1 wt %, or about 0.5 wt %.

55. The solid pharmaceutical composition or the process for making a solid pharmaceutical composition according to any of embodiments 1-54, wherein the at least one pharmaceutical excipient, the at least one intragranular pharmaceutical excipient or the at least one extragranular pharmaceutical excipient comprises or further comprises a lubricant.

56. The solid pharmaceutical composition or the process for making a solid pharmaceutical composition according to embodiment 55, wherein the lubricant comprises or is selected from a fatty acid, a fatty acid salt, a fatty acid ester, talc, a glyceride ester, a metal silicate, or any combination thereof.

57. The solid pharmaceutical composition or the process for making a solid pharmaceutical composition according to embodiment 55, wherein the lubricant comprises or is selected from magnesium stearate, stearic acid, magnesium silicate, aluminum silicate, isopropyl myristate, sodium oleate, sodium stearoyl lactate, sodium stearoyl fumarate, titanium dioxide, or combinations thereof.

58. The solid pharmaceutical composition or the process for making a solid pharmaceutical composition according to embodiment 55, wherein the lubricant comprises or is magnesium stearate.

59. The solid pharmaceutical composition or the process for making a solid pharmaceutical composition according to any of embodiments 55-58, wherein the total concentration of the lubricant in the solid pharmaceutical composition is from 0.05 wt % to 5 wt %, 0.1 wt % to 3 wt %, 1 wt % to 2 wt %, or about 1.5 wt %.

60. The solid pharmaceutical composition or the process for making a solid pharmaceutical composition according to any of embodiments 1-59, wherein the solid pharmaceutical composition is a mini-tablet.

61. The solid pharmaceutical composition or the process for making a solid pharmaceutical composition according to embodiment 60, wherein the mini-tablet is in the form of a solid cylinder having a cylindrical axis, a cylindrical side face, circular end faces perpendicular to the cylindrical axis, a diameter across the circular end faces, and a length along the cylindrical side face.

62. The solid pharmaceutical composition or the process for making a solid pharmaceutical composition according to embodiment 61, wherein length of the mini-tablet exceeds the diameter of the mini-tablet to provide the mini-tablet with an aspect ratio (length:diameter) of greater than 1:1.

63. The solid pharmaceutical composition or the process for making a solid pharmaceutical composition according to any of embodiments 61-62, wherein the mini-tablet has a diameter of from 1.0 mm to 3.2 mm, or from 1.5 mm to 3.1 mm.

64. The solid pharmaceutical composition or the process for making a solid pharmaceutical composition according to any of embodiments 61-63, wherein the mini-tablet has a length of from 1.7 mm to 4.8 mm, or from 2.0 mm to 4.5 mm.

65. A solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin in a concentration of 10 wt % of the solid pharmaceutical composition;
(c) meglumine in a concentration of 1 wt % of the solid pharmaceutical composition;
(d) microcrystalline cellulose in a concentration of 10 wt % of the solid pharmaceutical composition;
(e) anhydrous dibasic calcium phosphate in a concentration of 19 wt % of the solid pharmaceutical composition;
(f) vinylpyrrolidone-vinyl acetate copolymer in a concentration of 8 wt % of the solid pharmaceutical composition;
(g) colloidal silicon dioxide in a concentration of 0.5 wt % of the solid pharmaceutical composition; and
(h) magnesium stearate in a concentration of 1.50 wt % of the solid pharmaceutical composition; or
a solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin;
(c) meglumine;
(d) microcrystalline cellulose;
(e) anhydrous dibasic calcium phosphate;
(f) vinylpyrrolidone-vinyl acetate copolymer;
(g) colloidal silicon dioxide; and
(h) magnesium stearate.

66. A solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin in a concentration of 10 wt % of the solid pharmaceutical composition;
(c) meglumine in a concentration of 1 wt % of the solid pharmaceutical composition;
(d) microcrystalline cellulose in a concentration of 24.5 wt % of the solid pharmaceutical composition;
(e) silicified microcrystalline cellulose in a concentration of 6.0 wt % of the solid pharmaceutical composition;
(f) vinylpyrrolidone-vinyl acetate copolymer in a concentration of 6.0 wt % of the solid pharmaceutical composition;
(g) colloidal silicon dioxide in a concentration of 0.5 wt % of the solid pharmaceutical composition; and
(h) magnesium stearate in a concentration of 2.0 wt % of the solid pharmaceutical composition; or
a solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin;
(c) meglumine;
(d) microcrystalline cellulose;
(e) silicified microcrystalline cellulose;
(f) vinylpyrrolidone-vinyl acetate copolymer;
(g) colloidal silicon dioxide; and
(h) magnesium stearate.

67. A solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin in a concentration of 10 wt % of the solid pharmaceutical composition;
(c) meglumine in a concentration of 1 wt % of the solid pharmaceutical composition;
(d) hydroxypropyl methylcellulose in a concentration of 1.5 wt % of the solid pharmaceutical composition;
(e) mannitol in a concentration of 21.0 wt % of the solid pharmaceutical composition;
(f) sodium lauryl sulfate in a concentration of 0.25 wt % of the solid pharmaceutical composition;
(g) microcrystalline cellulose in a concentration of 7.25 wt % of the solid pharmaceutical composition;
(h) vinylpyrrolidone-vinyl acetate copolymer in a concentration of 7.25 wt % of the solid pharmaceutical composition;
(i) colloidal silicon dioxide in a concentration of 0.25 wt % of the solid pharmaceutical composition; and
(j) magnesium stearate in a concentration of 1.50 wt % of the solid pharmaceutical composition; or
a solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin;
(c) meglumine;
(d) hydroxypropyl methylcellulose;
(e) mannitol;
(f) sodium lauryl sulfate;
(g) microcrystalline cellulose;
(h) vinylpyrrolidone-vinyl acetate copolymer;
(i) colloidal silicon dioxide; and
(j) magnesium stearate.

68. A solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin in a concentration of 10 wt % of the solid pharmaceutical composition;
(c) meglumine in a concentration of 1 wt % of the solid pharmaceutical composition;
(d) microcrystalline cellulose in a concentration of 17.5 wt % of the solid pharmaceutical composition;
(e) silicified microcrystalline cellulose in a concentration of 10.75 wt % of the solid pharmaceutical composition;
(f) vinylpyrrolidone-vinyl acetate copolymer in a concentration of 7.5 wt % of the solid pharmaceutical composition;
(g) colloidal silicon dioxide in a concentration of 0.25 wt % of the solid pharmaceutical composition;
(h) hydroxypropyl methylcellulose in a concentration of 1.5 wt % of the solid pharmaceutical composition; and
(i) magnesium stearate in a concentration of 1.5 wt % of the solid pharmaceutical composition; or
a solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin;
(c) meglumine;
(d) microcrystalline cellulose;
(e) silicified microcrystalline cellulose;
(f) vinylpyrrolidone-vinyl acetate copolymer;
(g) colloidal silicon dioxide;
(h) hydroxypropyl methylcellulose; and
(i) magnesium stearate.

69. A process for making a solid pharmaceutical composition comprising:
(a) preparing an intragranular solid composition by a roller compaction process, the intragranular solid composition consisting essentially of:
   (i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
   (ii) hydroxypropyl-beta-cyclodextrin in a concentration of 10 wt % of the solid pharmaceutical composition;
   (iii) meglumine in a concentration of 1 wt % of the solid pharmaceutical composition;
   (iv) microcrystalline cellulose in a concentration of 10 wt % of the solid pharmaceutical composition;
   (v) vinylpyrrolidone-vinyl acetate copolymer in a concentration of 8 wt % of the solid pharmaceutical composition; and
   (vi) magnesium stearate in a concentration of 0.75 wt % of the solid pharmaceutical composition;
(b) combining the intragranular solid composition with extragranular components to form a blend, wherein the extragranular components consist essentially of:
   (i) anhydrous dibasic calcium phosphate in a concentration of 19 wt % of the solid pharmaceutical composition;
   (ii) colloidal silicon dioxide in a concentration of 0.5 wt % of the solid pharmaceutical composition; and
   (iii) magnesium stearate in a concentration of 0.75 wt % of the solid pharmaceutical composition; and
(c) tableting the blend to form of a solid pharmaceutical composition in the form of mini-tablets.

70. A process for making a solid pharmaceutical composition comprising:
(a) preparing an intragranular solid composition by a roller compaction process, the intragranular solid composition consisting essentially of:
   (i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
   (ii) hydroxypropyl-beta-cyclodextrin in a concentration of 10 wt % of the solid pharmaceutical composition;
   (iii) meglumine in a concentration of 1 wt % of the solid pharmaceutical composition;
   (iv) microcrystalline cellulose in a concentration of 24.5 wt % of the solid pharmaceutical composition;
   (v) colloidal silicon dioxide in a concentration of 0.2 wt % of the solid pharmaceutical composition; and
   (vi) magnesium stearate in a concentration of 0.75 wt % of the solid pharmaceutical composition.
(b) combining the intragranular solid composition with extragranular components to form a blend, wherein the extragranular components consist essentially of:
   (i) silicified microcrystalline cellulose in a concentration of 6.0 wt % of the solid pharmaceutical composition;
   (ii) vinylpyrrolidone-vinyl acetate copolymer in a concentration of 6.0 wt % of the solid pharmaceutical composition;
   (iii) colloidal silicon dioxide in a concentration of 0.3 wt % of the solid pharmaceutical composition; and
   (iv) magnesium stearate in a concentration of 1.25 wt % of the solid pharmaceutical composition; and
(c) tableting the blend to form of a solid pharmaceutical composition in the form of mini-tablets.

71. A process for making a solid pharmaceutical composition comprising:
(a) preparing an intragranular solid composition by a fluid bed granulation process, the intragranular solid composition consisting essentially of:
   (i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
   (ii) hydroxypropyl-beta-cyclodextrin in a concentration of 10 wt % of the solid pharmaceutical composition;
   (iii) meglumine in a concentration of 1 wt % of the solid pharmaceutical composition;
   (iv) mannitol in a concentration of 21 wt % of the solid pharmaceutical composition;
   (v) sodium lauryl sulfate in a concentration of 0.25 wt % of the solid pharmaceutical composition; and
   (vi) hydroxypropyl methylcellulose in a concentration of 1.5 wt % of the solid pharmaceutical composition;
(b) combining the intragranular solid composition with extragranular components to form a blend, wherein the extragranular components consist essentially of:
   (i) microcrystalline cellulose in a concentration of 7.25 wt % of the solid pharmaceutical composition;
   (ii) vinylpyrrolidone-vinyl acetate copolymer in a concentration of 7.25 wt % of the solid pharmaceutical composition;
   (iii) colloidal silicon dioxide in a concentration of 0.25 wt % of the solid pharmaceutical composition; and
   (iv) magnesium stearate in a concentration of 1.50 wt % of the solid pharmaceutical composition; and
(c) tableting the blend to form of a solid pharmaceutical composition in the form of mini-tablets.

72. A process for making a solid pharmaceutical composition comprising:
(a) preparing an intragranular solid composition by a fluid bed granulation process, the intragranular solid composition consisting essentially of:
   (i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
   (ii) hydroxypropyl-beta-cyclodextrin in a concentration of 10 wt % of the solid pharmaceutical composition;
   (iii) meglumine in a concentration of 1 wt % of the solid pharmaceutical composition;
   (iv) microcrystalline cellulose in a concentration of 10 wt % of the solid pharmaceutical composition; and
   (v) hydroxypropyl methylcellulose in a concentration of 1.5 wt % of the solid pharmaceutical composition;
(b) combining the intragranular solid composition with extragranular components to form a blend, wherein the extragranular components consist essentially of:
   (i) microcrystalline cellulose in a concentration of 7.5 wt % of the solid pharmaceutical composition; and
   (ii) vinylpyrrolidone-vinyl acetate copolymer in a concentration of 7.5 wt % of the solid pharmaceutical composition;
   (iii) silicified microcrystalline cellulose in a concentration of 10.75 wt % of the solid pharmaceutical composition;
   (iv) colloidal silicon dioxide in a concentration of 0.25 wt % of the solid pharmaceutical composition; and
   (iv) magnesium stearate in a concentration of 1.5 wt % of the solid pharmaceutical composition; and
(c) tableting the blend to form of a solid pharmaceutical composition in the form of mini-tablets.

73. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, wherein the first material comprises a polycarbonate-based aromatic thermoplastic polyurethane and the second material comprises an aliphatic polyether-based thermoplastic polyurethane; and
a drug formulation disposed in the closed drug reservoir lumen, the drug formulation comprising a drug,
wherein (i) the second wall structure, or both the first wall structure and the second wall structure, are permeable to water, and (ii) the first wall structure is impermeable to the drug and the second wall structure is permeable to the drug, such that the drug is releasable in vivo by diffusion through the second material forming the second wall structure.

74. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, wherein the first material comprises an aromatic polyester hydrocarbon-based thermoplastic polyurethane and the second material comprises an aliphatic polyether-based thermoplastic polyurethane; and
a drug formulation disposed in the closed drug reservoir lumen, the drug formulation comprising a drug,
wherein (i) the second wall structure, or both the first wall structure and the second wall structure, are permeable to water, and (ii) the first wall structure is impermeable to the drug and the second wall structure is permeable to the drug, such that the drug is releasable in vivo by diffusion through the second wall structure.

75. The drug delivery system of either one of embodiments 73 and 74, wherein the second wall structure forms a longitudinal strip extending along the length of the tube.

76. The system of any one of embodiments 73-75, wherein the system is configured to release a therapeutically effective amount of the drug at a substantially zero order release rate over at least 36 hours.

77. The system of any one of embodiments 73-76, wherein the system is configured to release the drug over a period of 2 days to 6 months.

78. The system of any one of embodiments 73-77, wherein the two interface edges are disposed at an arc angle of from 15 degrees to 270 degrees of a circumference of the tube in a cross section normal to a longitudinal axis of the tube.

79. The system of any one of embodiments 73-78, wherein the drug comprises erdafitinib, in particular is erdafitinib.

80. The system of embodiment 79, wherein the system is configured to release the erdafitinib at an average rate of 1 mg/day to 10 mg/day.

81. The system of embodiment 79, wherein the system is configured to release the erdafitinib at an average rate of 1 mg/day to 2 mg/day.

82. The system of embodiment 81, wherein the two interface edges are disposed at an arc angle of 45 degrees to 90 degrees of a circumference of the tube in a cross section normal to a longitudinal axis of the tube.

83. The system of embodiment 79, wherein the system is configured to release the erdafitinib at an average rate of 4 mg/day to 6 mg/day.

84. The system of embodiment 83, wherein the two interface edges are disposed at an arc angle of 150 degrees to 270 degrees of a circumference of the tube in a cross section normal to a longitudinal axis of the tube.

85. The system of embodiment 79, wherein the system is configured to release the erdafitinib at an average rate of 1 mg/day.

86. The system of embodiment 85, wherein the two interface edges are disposed at an arc angle of about 45 degrees of a circumference of the tube in a cross section normal to a longitudinal axis of the tube.

87. The system of embodiment 79, wherein the system is configured to release the erdafitinib at an average rate of 2 mg/day.

88. The system of embodiment 87, wherein the two interface edges are disposed at an arc angle of about 90 degrees of a circumference of the tube in a cross section normal to a longitudinal axis of the tube.

89. The system of embodiment 79, wherein the system is configured to release the erdafitinib at an average rate of 4 mg/day.

90. The system of embodiment 89, wherein the two interface edges are disposed at an arc angle of about 180 degrees of a circumference of the tube in a cross section normal to a longitudinal axis of the tube.

91. The system of embodiment 79, wherein the system is configured to release the erdafitinib at an average rate of 6 mg/day.

92. The system of embodiment 91, wherein the two interface edges are disposed at an arc angle of 210 degrees to 270 degrees of a circumference of the tube in a cross section normal to a longitudinal axis of the tube.

93. The system of any one of embodiments 79-92, wherein the system comprises 500 mg of the erdafitinib.

94. The system of any one of embodiments 73-93, wherein a release profile of the drug is substantially independent of pH over a pH range of 5 to 7.

95. The system of any one of embodiments 73-94, wherein the second wall structure comprises less than 50 percent of a cross sectional area of the tube, in a cross section normal to the longitudinal axis of the tube.

96. The system of any one of embodiments 73-94, wherein the second wall structure comprises less than 25 percent of a cross sectional area of the tube, in a cross section normal to the longitudinal axis of the tube.

97. The system of any one of embodiments 73-96, wherein the tube has a substantially constant thickness over its circumference.

98. The system of any one of embodiments 73-97, further comprising a pair of end plugs and/or an adhesive material that seal the ends of the tube.

99. The system of any one of embodiments 73-98, wherein the first and second wall structures are integrally formed.

100. The system of embodiment 99, wherein the tube is formed in an extrusion process.

101. The system of any one of embodiments 73-100, wherein the system is elastically deformable between a relatively straightened deployment shape suited for insertion through the urethra of a patient and into the patient's bladder and a retention shape suited to retain the system within the bladder.

102. The system of any one of embodiments 73-101, wherein the system is elastically deformable and comprises overlapping curls formed by the tube, and the tube has two opposing free ends, which are directed away from one another when the system is in a low-profile deployment shape and which are directed toward one another when the system is in a relatively expanded retention shape.

103. The system of any one of embodiments 73-102, wherein the system is elastically deformable and has a bi-oval retention shape, and the tube has two opposing free ends which lie within an outer boundary of the bi-oval retention shape.

104. The system of any one of embodiments 73-103, further comprising a retention frame lumen.

105. The system of embodiment 104, further comprising a nitinol wire disposed in the retention frame lumen.

106. The system of any one of embodiments 73-105, wherein the first material has a Shore durometer value from 70A to 80A.

107. The system of any one of embodiments 73-106, wherein the second material has a Shore durometer value from 70A to 75A.

108. The system of any one of embodiments 73-107, wherein the drug formulation comprises the solid pharmaceutical composition of any one of embodiments 1, 2, 3, and 14-68.

109. The system of any one of embodiments 73-108, wherein the drug formulation is in the form of a plurality of mini-tablets serially arranged in the drug lumen.

110. The system of embodiment 109, wherein the plurality of mini-tablets comprise the mini-tablets of any one of embodiments 60-64.

111. A drug delivery system, comprising:
a housing defining a drug reservoir lumen bounded by a first wall structure formed of a first material and a second wall structure formed of a second material, wherein the first material comprises a polycarbonate-based aromatic thermoplastic polyurethane and the second material comprises an aliphatic polyether-based thermoplastic polyurethane; and
a drug formulation disposed in the drug reservoir lumen, the drug formulation comprising erdafitinib,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second material forming the second wall structure.

112. The system of embodiment 111, wherein the first and second wall structures are adjacent one another at two interface edges and together form a tube, and (i) the system is configured to release the erdafitinib at an average rate of 2 mg/day and the two interface edges are disposed at an arc angle of about 90 degrees of a circumference of the tube in a cross section normal to a longitudinal axis of the tube, (ii) the system is configured to release the erdafitinib at an average rate of 4 mg/day and the two interface edges are disposed at an arc angle of about 180 degrees of a circumference of the tube in a cross section normal to a longitudinal axis of the tube, or (iii) the system is configured to release the erdafitinib at an average rate of 6 mg/day and the two interface edges are disposed at an arc angle of 240 degrees.

113. A drug delivery system, comprising:
a housing defining a drug reservoir lumen bounded by a first wall structure formed of a first material and a second wall structure formed of a second material, wherein the first material comprises an aromatic polyester hydrocarbon-based thermoplastic polyurethane and the second material comprises an aliphatic polyether-based thermoplastic polyurethane; and
a drug formulation disposed in the closed drug reservoir lumen, the drug formulation comprising erdafitinib,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second wall structure.

114. The system of embodiment 113, wherein the first and second wall structures are adjacent one another at two interface edges and together form a tube, and (i) the system is configured to release the erdafitinib at an average rate of 2 mg/day and the two interface edges are disposed at an arc angle of about 90 degrees of a circumference of the tube in a cross section normal to a longitudinal axis of the tube, or (ii) the system is configured to release the erdafitinib at an average rate of 4 mg/day and the two interface edges are disposed at an arc angle of about 180 degrees of a circumference of the tube in a cross section normal to a longitudinal axis of the tube.

115. The system of any one of embodiments 111-114, wherein the system is elastically deformable and comprises overlapping curls formed by the tube, and the tube has two opposing free ends, which are directed away from one another when the system is in a low-profile deployment shape and which are directed toward one another when the system is in a relatively expanded retention shape.

116. The systems of any one of embodiments 111-115, wherein a release profile of the erdafitinib is substantially independent of pH over a pH range of 5 to 7.

117. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, with the second wall structure forming a longitudinal strip extending along the length of the tube, wherein the first material comprises a polycarbonate-based aromatic thermoplastic polyurethane and the second material comprises an aliphatic polyether-based thermoplastic polyurethane; and
a drug formulation disposed in the closed drug reservoir lumen, the drug formulation comprising the solid pharmaceutical composition of any one of embodiments 1, 2, 3 and 14-68,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second material forming the second wall structure,
wherein the system is configured to release a therapeutically effective amount of the erdafitinib at a substantially zero order release rate over at least 3 days, and
wherein (i) the system is configured to release the erdafitinib at an average rate of 2 mg/day and the two interface edges are disposed at an arc angle of about 90 degrees of a circumference of the tube in a cross section normal to a longitudinal axis of the tube, (ii) the system is configured to release the erdafitinib at an average rate of 4 mg/day and the two interface edges are disposed at an arc angle of about 180 degrees of a circumference of the tube in a cross section normal to a longitudinal axis of the tube, or (iii) the system is configured to release the erdafitinib at an average rate of 6 mg/day and the two interface edges are disposed at an arc angle of 240 degrees.

118. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, with the second wall structure forming a longitudinal strip extending along the length of the tube, wherein the first material comprises an aromatic polyester hydrocarbon-based thermoplastic polyurethane and the second material comprises an aliphatic polyether-based thermoplastic polyurethane; and
a drug formulation disposed in the closed drug reservoir lumen, the drug formulation comprising the solid pharmaceutical composition of any one of embodiments 1, 2, 3, and 14-68,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second wall structure,
wherein the system is configured to release a therapeutically effective amount of the erdafitinib at a substantially zero order release rate over at least 3 days, and
wherein (i) the system is configured to release the erdafitinib at an average rate of 2 mg/day and the two interface edges are disposed at an arc angle of about 90 degrees of a circumference of the tube in a cross section normal to a longitudinal axis of the tube, or (ii) the system is configured to release the erdafitinib at an average rate of 4 mg/day and the two interface edges are disposed at an arc angle of about 180 degrees of a circumference of the tube in a cross section normal to a longitudinal axis of the tube.

119. A method of treatment of bladder cancer, comprising locally delivering erdafitinib into the bladder of a patient in need thereof, in an amount effective for the treatment of bladder cancer.

120. The method of embodiment 119, wherein the bladder cancer is muscle invasive bladder cancer.

121. The method of embodiment 119, wherein the bladder cancer is non-muscle invasive bladder cancer.

122. The method of embodiment 119, wherein the bladder cancer is bacillus calmette-guérin (BCG)-naïve.

123. The method of any one of embodiments 119-122, wherein the erdafitinib is in the form of the solid pharmaceutical composition of any one of embodiments 1, 2, 3 and 14-68.

124. A method of intravesical administration of erdafitinib, comprising:
deploying an intravesical system into the bladder of a patient, the system comprising the solid pharmaceutical composition of any one of embodiments 1, 2, 3, and 14-68; and
releasing the erdafitinib from the system.

125. The method of embodiment 124, wherein the intravesical system is the drug delivery system of any one of embodiments 73-118, and releasing the erdafitinib from the system comprises releasing the erdafitinib from the drug reservoir lumen via diffusion through the second wall structure.

126. The method of either of embodiments 124 or 125, wherein the system is elastically deformed into a low-profile deployment shape and inserted through the urethra and into the patient's bladder, and then assumes a relatively expanded retention shape within the bladder.

127. A drug delivery system comprising:
a device configured for intravesical deployment; and
a drug formulation disposed within the device and comprising erdafitinib,
wherein the system is configured to release the erdafitinib from the device following intravesical deployment of the drug delivery system.

128. The drug delivery system of embodiment 127, wherein the drug formulation comprises a plurality of tablets which comprise the erdafitinib.

129. The drug delivery system of embodiment 128, wherein the tablets comprise the solid pharmaceutical composition of any one of embodiments 1, 2, 3 and 14-68.

130. The drug delivery system of any one of embodiments 127 to 129, wherein the system is configured to release the erdafitinib by diffusion through a drug permeable portion of the device.

131. The drug delivery system of any one of embodiments 127 to 130, wherein the system is configured to release the erdafitinib at a release rate from about 1 mg/day to about 6 mg/day, such as 2 to 4 mg/day.

132. A method of treating non-muscle invasive bladder cancer (NMIBC) or muscle invasive bladder cancer (MIBC) in a cancer patient, comprising:
locally delivering a therapeutically effective amount of erdafitinib into the bladder of the patient.

133. The method of embodiment 132, wherein the locally delivering erdafitinib comprises releasing the erdafitinib from an intravesical system at a release rate from about 1 mg/day to about 6 mg/day, such as 2 to 4 mg/day.

134. The method of embodiment 133, wherein the intravesical system is maintained in the patient's bladder for up to 90 days, and then, optionally, replaced with another erdafitinib-releasing intravesical system.

135. A method of treating (i) recurrent, non-muscle-invasive or muscle-invasive urothelial carcinoma of the bladder, (ii) high- or intermediate-risk papillary urothelial carcinoma of the bladder, or (iii) muscle-invasive urothelial carcinoma of the bladder staged cT2-T3a in a cancer patient, comprising:
locally delivering a therapeutically effective amount of erdafitinib into the bladder of the patient.

136. The method embodiment 131, wherein the patient undergoes transurethral resection of bladder tumor (TURBT) to reduce the total tumor(s) size to less than or equal to 3 cm, prior to the locally delivering of the erdafitinib into the bladder.

137. The method of embodiment 135 or 136, wherein the locally delivering erdafitinib comprises releasing the erdafitinib from an intravesical system at a release rate from about 1 mg/day to about 6 mg/day, such as 2 to 4 mg/day.

138. The method of embodiment 137, wherein the intravesical system is maintained in the patient's bladder for up to 90 days, and then, optionally, replaced with another erdafitinib-releasing intravesical system.

139. A method of treating a Bacillus Calmette-Guérin (BCG) experienced patient having recurrent high-grade Ta/T1 urothelial carcinoma of the bladder within 18 months of completion of prior BCG therapy, comprising:
locally delivering a therapeutically effective amount of erdafitinib into the bladder of the patient.

140. The method of embodiment 139, wherein the locally delivering erdafitinib comprises releasing the erdafitinib from an intravesical system at a release rate from about 1 mg/day to about 6 mg/day, such as 2 to 4 mg/day.

141. The method of embodiment 140, wherein the intravesical system is maintained in the patient's bladder for up to 90 days, and then, optionally, replaced with another erdafitinib-releasing intravesical system.

142. The method of any one of embodiments 132 to 141, wherein the erdafitinib is locally delivered into the bladder from a drug delivery system according to any one of embodiments 127 to 131.

143. The method of any one of embodiments 132 to 142, wherein the patient harbors at least one FGFR2 genetic alteration and/or FGFR3 genetic alteration.

### EXAMPLES

The examples below are intended to be purely exemplary of the invention and should therefore not be considered to limit the invention in any way. The following examples and detailed description are offered by way of illustration and not by way of limitation.

Organ-confined bladder cancer is a global unmet need as reflected by high morbidity and limited improvements in treatment over the past 2 decades. Globally, bladder cancer is the sixth and the 17th most commonly occurring cancer in men and women, respectively. There were nearly 550,000 new cases of bladder cancer diagnosed worldwide in 2018. Most bladder cancers are initially diagnosed in the early stages of the disease with 70% to 75% presenting as non-muscle invasive bladder cancer (NMIBC) and 25% to 30% as muscle invasive bladder cancer (MIBC).

Progression of the disease is a devastating life-changing event that often results in bladder removal for patients eligible for surgery. Following surgery, and for a large proportion of patients who are unfit for surgery, tumors often reoccur and progress to metastatic disease where the 5-year survival rate is 5%. New therapies are particularly difficult to develop because only a small fraction of systemically administered agents reach the tumors located in the urothelium. Therefore, there is a significant need for new targeted therapies to treat early disease and prevent progression to the invasive forms of bladder cancer.

### Example 1. Sample Minitablet Formulations

Table 1 illustrates selected aspects and embodiments of the minitablet formulation for use with the disclosed drug delivery systems. The following tablets were prepared with an 11.5 mg API drug load, to provide a 23.0 mg tablet.

TABLE 1
Exemplary erdafitinib minitablet formulations

| | | | |
|---|---|---|---|
| Formula 4A | Formula 4B | Formula 4C | Formula 4D |

| | |
|---|---|
| Component | Weight % |

Intragranular

| | | | | |
|---|---|---|---|---|
| Erdafitinib API | 50.00 | 50.00 | 50.00 | 50.00 |
| Hydroxypropyl-β-cyclodextrin | 10.00 | 10.00 | 10.00 | 10.00 |
| Meglumine | 1.00 | 1.00 | 1.00 | 1.00 |
| Mannitol | | | 21.00 | |
| Microcrystalline cellulose, MCC | 10.00 | 23.50 | | 10.00 |
| (e.g., Avicel ® PH105) | | | | |
| vinylpyrrolidone-vinyl acetate | 8.00 | | | |
| copolymer (e.g., Kollidon ® VA64) | | | | |
| Colloidal silica (e.g., Aerosil ® 200) | | 0.20 | | |
| Sodium Lauryl Sulfate, SLS | | | 0.25 | |
| Hydroxypropyl methylcellulose, | | | 1.50 | 1.50 |
| HPMC | | | | |
| Mg Stearate (e.g., Ligamed ® MF- | 0.75 | 0.75 | | |
| 2V) | | | | |

Extragranular

| | | | | |
|---|---|---|---|---|
| Dibasic calcium phosphate | 19.00 | | | |
| Silicified microcrystalline cellulose, | | 6.00 | | 10.75 |
| SMCC | | | | |
| (e.g., Prosolv ® HD90) | | | | |
| vinylpyrrolidone-vinyl acetate | | 6.00 | 7.25 | 7.50 |
| copolymer (e.g., Kollidon ® VA64) | | | | |
| Colloidal silica (e.g., Aerosil ® 200) | 0.50 | 0.30 | 0.25 | 0.25 |
| Microcrystalline cellulose, MCC | | | 7.25 | 7.50 |
| (e.g., Avicel ® PH105) | | | | |
| Mg Stearate (e.g., Ligamed ® MF- | 0.75 | 1.25 | 1.50 | 1.50 |
| 2V) | | | | |
| Total | 100% | 100% | 100% | 100% |

The intragranular solid composition of Formula 4A and Formula 4B were prepared by a roller compaction process. The intragranular solid composition of Formula 4C and Formula 4D were prepared by a fluid bed granulation process.

In one aspect, the exemplary formulations illustrated here can be prepared by any method. For example, the intragranular solid composition can be prepared by a roller compaction process, by a fluid bed granulation process, or other processes. In one aspect, the intragranular solid composition of Formula 4A and Formula 4B can be prepared by a roller compaction process. In a further aspect, the intragranular solid composition of Formula 4C and Formula 4D can be prepared by a fluid bed granulation process.

### Example 2. Tablet Manufacturing Evaluation of Roller Compaction Versus Fluid Bed Granulation Methods

A number of manufacturing aspects were examined and the roller compaction (RC) versus fluid bed granulation (FBG) concepts were compared for tablet manufacture. Certain performance characteristics or standards are necessary for a formulation to be amenable to high speed tableting, as demonstrated in this example.

Tablet ejection forces arising from roller compaction (RC) and fluid bed granulation (FBG) methods were examined. For the FBG method, lower ejection forces were observed. In RC tablets, ejection forces were higher, which imposes a potential risk of process interruptions, in particular in case of high speed tableting.

The in-process controls, while in-spec for both RC and FBG methods, showed more variability (higher relative standard deviation, RSD) in the RC tablets.

Higher production speeds were found to be more accessible in the FBG batches. For example, FBG tablet batches were capable of being run at high speed, about 2500 tabs/min (tablets per minute) or 6 hours/20 kg run. In contrast, the RC tablet batches were capable of medium speeds, about 1800 tabs/min or 9 hours/20 kg run, as the higher production speeds were not achievable due to the higher ejection forces.

The FBG process was generally a more robust method as compared with the RC process. No flashing or tablet defects were noticed in the FBG tablet batches, whereas the RC tablets tended to break more easily and “round off” during system builds, and flashing was noticed towards end of run with the RC tablets.

The Formula 4A and 4B formulation tablets described in the Table 1 were prepared by a roller compaction (RC) process. The Formula 4C and 4D formulation tablets described in the Table 1 were prepared by a fluid bed granulation (FBG) process. In contrast to Formula 4A and Formula 4B tablets, the Formula 4C and Formula 4D fluid bed granulation (FBG) formulation tablets could be produced at higher production speeds due to the lower ejection forces, and the resulting tablets were more robust and less susceptible to breakage.

The Formula 4A formulation tablets were prepared by a RC process as follows:

1. The following components of the intragranular phase were pre-blended: microcrystalline cellulose; erdafitinib, hydroxypropylbeta cyclodextrin, meglumine, and anhydrous colloidal silica.

2. The pre-blend from step 1 was screened.

3. The screened pre-blend was blended to a homogeneous blend using a suitable blender.

4. Screened magnesium stearate was blended to the blend and mixed to a homogeneous blend using a suitable blender.

5. Roller compaction was performed to obtain a granulate.

6. The following components of the extragranular phase were pre-blended: silicified microcrystalline cellulose, copovidone, anhydrous colloidal silica.

7. The pre-blend from step 6 was screened.

8. The screened mixture was added to the granulate and mixed to a homogeneous blend using a suitable blender.

9. Screened magnesium stearate was added to the blend and mixed to a homogeneous blend using a suitable blender.

10. The blend was compressed into minitablets using a suitable tablet press and the tablets were passed through a deduster and metal detector.

11. The minitablets were packed in a suitable packaging configuration.

The Formula 4B formulation tablets were prepared according to an analogous process to that of Formula 4A.

The Formula 4D formulation tablets were prepared by a fluid bed granulation process as follows:

1. The following screened components of the intragranular phase were pre-blended to a homogeneous blend using a suitable blender: erdafitinib, meglumine, hydroxypropylbetacyclodextrin, microcrystalline cellulose.

2. A binder solution was created by dissolving hypromellose 2910 15 mPa·s in water for injections until a clear solution was obtained without lumps.

3. Fluid bed granulation was performed.

4. The granules were screened using a suitable screen.

5. The following screened components were added to the granulate and mixed to a homogeneous blend using a suitable blender: copovidone, microcrystalline cellulose, silicified microcrystalline cellulose, anhydrous colloidal silica.

6. Screened magnesium stearate was added to the blend and mixed further using a suitable blender.

7. The blend was compressed into minitablets using a suitable tablet press and dedusted.

8. The minitablets were packed in a suitable packaging configuration.

The Formula 4C formulation tablets were prepared according to an analogous process to that of Formula 4D.

### Example 3. Further Exemplary Erdafitinib Formulations

The following erdafitinib formulations set out in Table 2 and Table 3 were prepared and examined with respect to suitability for tablet formation. Tablets based on these formulations were prepared with an 11.5 mg A-PI drug load, to provide a 23.0 mg tablet.

TABLE 2
Examples of erdafitinib formulations

| | | | |
|---|---|---|---|
| 1.1 | 2.1 | 2.2 | 3.1 |

| | |
|---|---|
| Component | Weight % |

Intragranular

| | | | | |
|---|---|---|---|---|
| Erdafitinib API | 50.00 | 50.00 | 50.00 | 50.00 |
| Hydroxypropyl-β-cyclodextrin | 10.00 | 10.00 | 10.00 | 10.00 |
| Meglumine | 1.00 | 1.00 | 1.00 | 1.00 |
| Mannitol | 21.00 | — | — | — |
| Microcrystalline cellulose, MCC | — | — | — | 10.00 |
| (e.g., Avicel ® PH105) | | | | |
| Hydroxypropyl methylcellulose, | 1.50 | 1.50 | 1.50 | 1.50 |
| HPMC | | | | |

Extragranular

| | | | | |
|---|---|---|---|---|
| Silicified microcrystalline cellulose, | — | — | 15.75 | — |
| SMCC | | | | |
| (e.g., Prosolv ® HD90) | | | | |
| vinylpyrrolidone-vinyl acetate | 7.375 | 17.875 | 10.00 | 12.875 |
| copolymer (e.g., Kollidon ® VA64) | | | | |
| Colloidal silica (e.g., Aerosil ® 200) | 0.25 | 0.25 | 0.25 | 0.25 |
| Microcrystalline cellulose, MCC | 7.375 | 17.875 | — | 12.875 |
| (e.g., Avicel ® PH105) | | | | |
| Mg Stearate (e.g., Ligamed ® MF- | 1.50 | 1.50 | 1.50 | 1.50 |
| 2V) | | | | |
| Total | 100% | 100% | 100% | 100% |

TABLE 3
Examples of erdafitinib formulations

| | | | |
|---|---|---|---|
| 3.2 | 3.3 | 3.4 | 4.1 |

| | |
|---|---|
| Component | Weight % |

Intragranular

| | | | | |
|---|---|---|---|---|
| Erdafitinib API | 50.00 | 50.00 | 50.00 | 50.00 |
| Hydroxypropyl-β-cyclodextrin | 10.00 | 10.00 | 10.00 | 10.00 |
| Meglumine | 1.00 | 1.00 | 1.00 | — |
| Mannitol | — | — | — | — |
| Microcrystalline cellulose, MCC | 10.00 | 10.00 | 10.00 | 10.00 |
| (e.g., Avicel ® PH105) | | | | |
| Hydroxypropyl methylcellulose, | 1.50 | 1.50 | 1.50 | 1.50 |
| HPMC | | | | |

Extragranular

| | | | | |
|---|---|---|---|---|
| Silicified microcrystalline cellulose, | 5.75 | 15.75 | 10.75 | 11.75 |
| SMCC | | | | |
| (e.g., Prosolv ® HD90) | | | | |
| vinylpyrrolidone-vinyl acetate | 10.00 | 10.00 | 7.50 | 7.50 |
| copolymer (e.g,. Kollidon ® VA64) | | | | |
| Colloidal silica (e.g., Aerosil ® 200) | 0.25 | 0.25 | 0.25 | 0.25 |
| Microcrystalline cellulose, MCC | 10.00 | — | 7.50 | 7.50 |
| (e.g., Avicel ® PH105) | | | | |
| Mg Stearate (e.g., Ligamed ® MF- | 1.50 | 1.50 | 1.50 | 1.50 |
| 2V) | | | | |
| Total | 100% | 100% | 100% | 100% |

Compression studies of the above formulations were conducted, using a fluid bed granulation process for each of the formulations.

The Component 4.1 formulation tablets were prepared by a fluid bed granulation process as follows:

1. The following screened components of the intragranular phase were pre-blended to a homogeneous blend using a suitable blender: erdafitinib, hydroxypropylbetacyclodextrin, microcrystalline cellulose.

2. A binder solution was created by dissolving hypromellose 2910 15 mPa·s in water for injections until a clear solution was obtained without lumps.

3. Fluid bed granulation was performed.

4. The granules were screened using a suitable screen.

5. The following screened components were added to the granulate and mixed to a homogeneous blend using a suitable blender: copovidone, microcrystalline cellulose, silicified microcrystalline cellulose, anhydrous colloidal silica.

6. Screened magnesium stearate was added to the blend and mixed further using a suitable blender.

7. The blend was compressed into minitablets using a suitable tablet press and dedusted.

8. The minitablets were packed in a suitable packaging configuration.

Data were also obtained for tablet hardness resulting from the intermittent pneumatic compression for each of formulation 1.1 through 4.1. Tablet thickness resulting from the intermittent pneumatic compression for each of formulation 1.1 through 4.1 was also studied. These data suggest that hardness and thickness of the tables are relatively consistently maintained for these formulations.

### Example 4. Process Monitoring Control of Compression Force and Ejection Force for Erdafitinib Formulations

Process monitoring control considerations of compression force and ejection force for erdafitinib test formulations were examined, and Formula 4B and Formula 4C were compared with the 1.1, 2.2, 3.4 and 4.1 formulations. (See Tables 2 and 3, above.)

### Example 5. Screening of Permeation Materials and API Forms for Erdafitinib Release

A number of polymeric materials were tested to determine their suitability as a material of construction of an elastic system body for release of various erdafitinib formulations in a permeation-controlled release system. The materials included silicone, several thermoplastic polyurethane (TPUs) manufactured by Lubrizol Life Science (Bethlehem, PA). The results are set forth in the Table 4.

TABLE 4
In Vitro Permeation

| | Erdafitinib free | Erdafitinib free | Erdafitinib HCl |
|---|---|---|---|
| Material | base | base + HP-β-CD | salt |
| Silicone (MED- | X | X | X |
| 4750) | | | |
| TPU (PC-3575A) | X | X | X |
| TPU (AR-62A) | X | X | X |
| TPU (HP-60D-35) | ◯ | ◯ | ◯ |
| TPU (AC-4075A) | X | X | X |
| TPU (TT-1074A) | X | Δ | X |
| TPU (HP-93A-100) | ◯ | ◯ | ◯ |
| TPU (EG-80A) | Δ | ◯ | Δ |
| EVA (CoTran 9712) | Δ | X | Δ |

In Table 4, “O” is permeable (suitable for the stripe material), “A” is practically impermeable, and “X” is impermeable (suitable for a base material, i.e., the non-stripe portion of a system body).

Based in part of the foregoing results, prototype system constituents were tested with erdafitinib free base and erdafitinib free base+10% HP-β-CD. The system constituents were extruded tubing of two materials (strip+base) and then assembled with certain of the erdafitinib formulations and release rates were tested in vitro in simulated urine at various pHs. The results are set forth in the Table 5.

TABLE 5
In Vitro Permeation Systems

| API Form | Device Constituent | Observations |
|---|---|---|
| erdafitinib free base + 10% | AR-62A base with EG-80A | Release profiles acceptable |
| HP-β-CD | stripe | |
| erdafitinib free base + 10% | AR-75A-B20 base with EG- | Release profiles acceptable, but |
| HP-β-CD | 80A stripe | AR-75A material was more |
| | | difficult to extrude than AC- |
| | | 4075A-B20 |
| erdafitinib free base + 10% | AR-62A base with HP-60D-35 | Release profile shows more pH |
| HP-β-CD | stripe | dependency |
| erdafitinib free base | EG-80A base with HP-60D-35 | Release profile shows more pH |
| | stripe | dependency |
| erdafitinib free base | AR-62A base with HP-60D-35 | Release profile shows more pH |
| | stripe | dependency |

Based on these results, a preferred permeation system appears to be a combination of a device constituent of AC-4075A-B20 (or AR-62A) base with EG-80A stripe with a drug formulation comprising erdafitinib base+10% HP-β-CD.

### Example 6. Erdafitinib Metabolism and Pharmacokinetic Properties for Intravesical Delivery

Erdafitinib was determined to be sufficiently stable in freshly collected human, minipig and rat urine at 37° C. for 6 hours, indicating that drug is stable in urine between void cycles.

In vitro protein binding indicated erdafitinib to be mainly present in free form in urine. Percent erdafitinib free in rat, minipig and human urine was assessed to be 84%, 97% and 95%, respectively and concentration independent. Erdafitinib found to bind to AGP less than to albumin. Albuminuria/proteinuria had minimal effect on % free form in urine.

Erdafitinib in vitro binding to normal/tumor bladder tissues showed significant free fraction. Free fraction of erdafitinib in minipig bladder tissue was 79%, 2 times higher than in rat (33%) and human (39%). In tumor tissue, free fraction was 40%.

Bladder perfusion studies in pig and rat showed good partitioning from urine into the bladder, and particularly into the urothelium. Low systemic bioavailability (˜5% in rat and 12% in minipig) after localized bladder dosing was observed.

Based on these studies, erdafitinib is stable in urine and present in high free fraction, which therefore should lead to desired exposures to bladder tumors. Erdafitinib appears to have favorable drug metabolism and pharmacokinetic properties for intravesical administration.

### Example 7. Erdafitinib+Cyclodextrin In Vitro Release Study

Erdafitinib co-formulated with cyclodextrin (HPPCD) was tested in systems having a base material of AC-4075A-B20 and stripe of EG-80A with a 90° stripe angle or a 1800 stripe angle in simulated urine at pH 5, pH 6.8, and pH 8. They exhibited minimal pH dependency at physiological pH (pH 5-7) and were able to achieve 90-day delivery at target rates of 2 to 4 mg/day.

### Example 8. Minipig Studies with Erdafitinib+Cyclodextrin

Prototype systems having the design described in Example 7 were tested in minipigs. Urine drug concentrations were maintained through 90 days. Average urine concentration: ˜1313 ng/mL (target>1190 ng/mL). Acceptable bladder local tolerability and no systemic toxicity were observed. The results were confirmed in 1-month minipig tolerability and GLP tox studies.

### Example 9. Human Study with Erdafitinib

A clinical study will be undertaken on human participants (patients) to evaluate the safety, pharmacokinetics (PK), and preliminary efficacy of an erdafitinib-releasing intravesical system in participants with intermediate- or high-risk papillary non-muscle invasive bladder cancer (NMIBC) or muscle invasive bladder cancer (MIBC) who have selected FGFR mutations or fusions.

### The Study

The study will utilize an erdafitinib intravesical delivery system (hereinafter “TAR-210”) according to the present invention that is to be retained in the bladder following insertion using a urinary placement catheter, where it will provide sustained release of erdafitinib for up to 90 days. The TAR-210 will be removed from the bladder transurethrally via cystoscopy and non-cutting endoscopic grasping forceps.

This open-label, multicenter, Phase 1 study of TAR-210 in adult participants with either NMIBC or MIBC will enroll 4 cohorts of participants:
Cohort 1: Recurrent, bacillus Calmette-Guerin (BCG)-experienced high-risk papillary-only NMIBC (high-grade Ta/T1), refusing or ineligible for radical cystectomy (RCy),
Cohort 2: Recurrent, BCG-experienced high-risk papillary-only NMIBC (high-grade Ta/T1), scheduled for RCy,
Cohort 3: Recurrent, intermediate-risk NMIBC (Ta and T1) with a previous history of only low-grade disease, and
Cohort 4: MIBC scheduled for RCy who have refused or are ineligible for cisplatin-based neoadjuvant chemotherapy.

For Cohorts 1 and 2, all visible tumor(s) must be completely resected prior to the start of study treatment and documented on screening cystoscopy. For Cohort 4, participants must have a total tumor size≤3 cm in order to be eligible.

The study will comprise 2 parts: Part 1 (dose escalation) and Part 2 (dose expansion). Part 1 dose escalation will include participants from Cohorts 1 and 3 and will be supported by a Bayesian Optimization Interval (BOIN) design. Two dose levels may be evaluated in this study: an intravesical delivery system with an estimated maximum erdafitinib release of approximately 2 mg/day and an intravesical delivery system with an estimated maximum erdafitinib release of approximately 4 mg/day. Dose escalation will be guided by a BOIN design with a target dose limiting toxicity (DLT) rate of ≤28%.

All participants will be screened for eligible FGFR mutations or fusions in tumor tissue. An eligible FGFR alteration must be identified prior to starting study treatment. Approximately 12 participants are planned for enrollment in Part 1 and 50 to 80 participants are planned for enrollment in Part 2 (15 to 25 per cohort for Cohorts 1, 3 and 4 across all dose levels tested, and no specific enrollment target for Cohort 2), up to a maximum of approximately 92 participants.

Once a preliminary RP2D has been cleared as safe by the Study Evaluation Team (SET), participants from all 4 cohorts may subsequently be enrolled in separate expansion cohorts at that dose level in Part 2 to further characterize the safety, PK, and preliminary antitumor activity. One or both dose levels may be expanded in Part 2 as an RP2D. Participants who are scheduled for RCy (Cohorts 2 and 4) will only be enrolled in Part 2, once initial safety and PK data are available in NMIBC participants from Cohorts 1 and 3. During the study, safety will be monitored by the SET at each dose escalation step and at regular intervals during dose expansion.

Clinical activity will be assessed using the following evaluations: cystoscopy, computed tomography (CT) or magnetic resonance (MR) urography, urine cytology, and pathologic assessment after biopsy/transurethral resection of the bladder tumor (TURBT) or RCy.

Blood samples and urine samples will be collected from participants at multiple timepoints to characterize the plasma and urine PK of erdafitinib. Bladder tissue will be collected, and the tissue PK of erdafitinib will be analyzed where feasible.

Safety assessments will be based on medical review of AE reports and the results of vital sign measurements, physical examinations, ophthalmological assessments, clinical laboratory tests, and other safety evaluations at specified timepoints. Concomitant medication usage will be recorded. Adverse events will be graded using National Cancer Institute Common Terminology Criteria for Adverse Events (NCI-CTCAE, Version 5.0).

Participants will have TAR-210 inserted transurethrally into the bladder using the Urinary Placement Catheter on Day 1 of the treatment phase. TAR-210 will be removed after 3 months (on Day 90) for Cohorts 1 and 3, or after 8 weeks (on Day 57) for Cohorts 2 and 4, or earlier in case of disease recurrence or progression or unacceptable toxicity. To mitigate the risk of urinary tract infections, participants may receive a dose of prophylactic periprocedural antibiotics for intravesical study procedures. Cystoscopy for removal of the TAR-210 may be used to facilitate the removal with a non-cutting grasping forceps, wherein the TAR-210 is grasped fully and removed from the bladder under direct vision, not through a cystoscope's working channel.

Following completion of the first 3-month dosing cycle, participants in Cohorts 1 and 3 will undergo disease response assessment with cystoscopy with biopsy and urine cytology; those participants with a complete response (CR) may continue to receive up to 3 additional 3-month dosing cycles with TAR-210, for a maximum treatment duration of 1 year if there is no disease recurrence or progression or unacceptable toxicity. Participants in Cohort 2 and Cohort 4 will undergo RCy after 8 weeks of dosing and will not receive further study treatment.

For all participants, an End of Treatment (EOT) visit will take place 30 (+7) days after removal of the last TAR-210 system. Participants in Cohorts 1 and 3 who have not recurred or progressed will enter a follow up phase and undergo cystoscopy, urine cytology, and upper tract imaging for up to 3 years after Day 1, or until disease recurrence or progression, a new anticancer therapy is initiated, or until the participant withdraws from the study. Participants in Cohorts 2 and 4 will have a follow-up visit 3 months after RCy. For Cohorts 1 and 3, after 90 days of study treatment (1 cycle) a disease evaluation including cystoscopy with biopsy of visible disease (or biopsy of previous sites of disease if no disease is visible) and urine cytology will be performed. Participants with complete response (CR) may continue to receive TAR-210 on 90-day cycles for up to 1-year total duration.

The study comprises molecular eligibility, screening, treatment, and follow-up phases.

Molecular eligibility will be established for each potential participant before screening for other eligibility criteria, unless a fresh tumor biopsy is required to obtain tissue for FGFR testing. Testing to document FGFR alterations may be performed on a fresh biopsy from recurrent disease, or an archived tumor tissue from prior to recurrence. An eligible FGFR alteration must be identified prior to start of study treatment.

High-risk NMIBC participants (Cohorts 1 and 2) must have a TURBT for their recurrent disease within 12 weeks prior to screening. For participants with lamina propria invasion (T1) on the screening biopsy/TURBT, muscularis propria must be present to rule out MIBC. All visible tumor(s) must be completely resected as documented by screening cystoscopy. MIBC participants (Cohort 4) must have a diagnostic TURBT within 12 weeks prior to the planned start of study treatment (Day 1) and must have a repeat TURBT if needed to reduce total tumor size to ≤3 cm within 8 weeks prior to Day 1, as per the Inclusion Criteria and schedule of activities. The last TURBT must be completed>14 days prior to Day 1. The intermediate-risk participants in Cohort 3 will not have a complete TURBT during screening or prior to starting study treatment.

The treatment phase will begin on Day 1 for participants meeting all eligibility criteria, at which time participants will have TAR-210 placed in the bladder with the urinary placement catheter. Following completion of the first dosing cycle, participants in Cohorts 1 and 3 will undergo biopsy and response will be assessed; those participants with a CR may continue to receive up to 3 additional 3-month dosing cycles with TAR-210, for a treatment duration of 1 year, so long as there is no disease recurrence or progression or unmanageable toxicity. Participants with MIBC (Cohort 4) and high-risk NMIBC in Cohort 2 will undergo RCy after 8 weeks of treatment and will not receive further study treatment.

Upon discontinuing treatment with TAR-210, participants will have an End of Treatment (EOT) visit (30 [+7] days after last system removal). Participants in Cohorts 2 and 4 will have a follow up visit 3 months (±2 weeks) after RCy. Participants in Cohorts 1 and 3 who have not recurred or progressed will enter a follow-up phase and will undergo disease surveillance with cystoscopy, urine cytology, and upper tract imaging for up to 3 years after Day 1, until disease recurrence or progression, a new anticancer therapy is initiated, or until the participant withdraws from the study.

Efficacy analyses will be performed separately for each cohort. The secondary endpoints for the 4 cohorts to assess preliminary clinical activity include RFS for Cohorts 1 and 2, CR rate and duration of CR for Cohort 3, CR rate, pT0 rate, and rate of downstaging to <pT2 for Cohort 4. Complete response for Cohorts 1* and 3 is defined as:
Negative cystoscopy and negative (including atypical) urine cytology
Positive cystoscopy with biopsy-proven benign or low-grade NMIBC and negative (including atypical) cytology
* It is noted that for Cohort 1, CR has the same operational definition as freedom from recurrence below.

Recurrence is defined as the histologically proven first appearance of high-grade Ta or T1 lesion bladder cancer after the start of study treatment (Cohorts 1 and 2) or after achievement of CR (Cohort 3). Freedom from recurrence for participants with NMIBC is defined as:
Negative cystoscopy and negative (including atypical) urine cytology.
Positive cystoscopy with biopsy-proven benign or low-grade NMIBC and negative (including atypical) cytology
Cohort 2 must be pT0 (no pathological evidence of intravesical disease) in the radical cystectomy pathology assessment

Pathologic complete response for Cohort 4 is defined as no pathologic evidence of intravesical disease (pT0) and no pathologic evidence of nodal involvement (pN0).

### Participant Inclusion/Exclusion Criteria

Each potential participant must satisfy all of the following criteria to be enrolled in the study:

### Age

1. ≥18 years (or the legal age of consent in the jurisdiction in which the study is taking place) at the time of informed consent.

### Type of Participant and Disease Characteristics

2. Recurrent, non-muscle-invasive or muscle-invasive urothelial carcinoma of the bladder.
   a. Mixed histology tumors are allowed if urothelial differentiation is predominant (ie, <20% variant histology). However, the presence of micropapillary, signet ring cell, plasmacytoid, neuroendocrine, or sarcomatoid features are exclusionary.
   b. High-risk papillary disease (Cohorts 1 [Parts 1 and 2] and 2 [Part 2 only]), defined as histologically confirmed high-grade Ta/T1 lesion. Concurrent CIS is not allowed. All visible tumor must be completely resected prior to the start of study treatment and documented on screening cystoscopy.
   c. Intermediate-risk papillary disease (Cohort 3, Parts 1 and 2) defined as all previous tumors being low grade, Ta or T1, and no previous CIS. Cystoscopic documentation of recurrence is sufficient. Negative urine cytology for high grade urothelial carcinoma is required.
   d. Muscle-invasive disease (Cohort 4, Part 2 only) cT2-T3a, NO. Participants must have a total tumor size≤3 cm after TURBT at assessment within 8 weeks prior to the start of study treatment or must have a second debulking TURBT to reduce the tumor(s) to ≤3 cm in order to be eligible.
3. Activating tumor FGFR mutation or fusion, as determined by local* or central testing, approved by the sponsor prior to the start of study treatment:
   * Local tissue-based results (if already existing) from next-generation sequencing (NGS) or polymerase chain reaction (PCR) tests performed in CLIA-certified or equivalent laboratories, or results from commercially available PCR or NGS tests.
4. Cohorts 1 and 2: BCG experienced, or participants with no BCG experience because BCG was not available as a treatment option in the participant's location within the previous 2 years and is currently unavailable. BCG experienced is defined as:
   Recurrent high-grade Ta/T1 disease within 18 months of completion of prior BCG therapy
   Prior BCG (minimum treatment requirernents):
      1) At least 5 of 6 full doses of an initial induction course. Full dose BCG defined as 1 full vial containing a minimum of 1×10⁸ colony forming units.
         OR
      2) At least 5 of 6 full doses of an initial induction course plus at least 1 maintenance (2 of 3 weekly doses) in a 6-month period. One-half dose or one-third dose is allowed during maintenance.
   Note: Cohort 3 has no predefined prior BCG or intravesical chemotherapy requirement.
5. Cohort 1 only: Refuses or is not eligible for RCy
6. Cohorts 2 and 4: Willing and eligible for RCy
7. Cohort 4: Refuses (and understands the risks and benefits of doing so) cisplatin-based combination chemotherapy or is deemed ineligible for cisplatin-based chemotherapy by meeting at least one of the following criteria:
   Creatinine clearance (CrCl)<60 mL/min
   NCI-CTCAE v.5.0 Grade≥2 audiometric hearing loss
   NCI-CTCAE v.5.0 Grade≥2 peripheral neuropathy
8. Eastern Cooperative Oncology Group (ECOG) performance status score of ≤2 (Cohorts 1 and 3) or ≤1 (Cohorts 2 and 4)
9. Adequate bone marrow, liver, and renal function:
   a. Bone marrow function (without the support of growth factors or transfusions in preceding 2 weeks):
      Absolute neutrophil count (ANC)≥1,000/mm³
      Platelet count≥75,000/mm³
      Hemoglobin≥8.0 g/dL
   b. Liver function:
      Total bilirubin≤1.5× the upper limit of normal (ULN) OR direct bilirubin≤1.5×ULN for participants with Gilbert's syndrome who have total bilirubin levels≥1.5×ULN
      Alanine aminotransferase (ALT) and aspartate aminotransferase (AST)≤2.5×ULN
   c. Renal function:
      Estimated glomerular filtration rate>30 mL/min calculated using the Modified Diet in Renal Disease (MDRD) formula

Any potential participant who meets any of the following criteria will be excluded from participating in the study:

### Medical Conditions

1. Concurrent extra-vesical (i.e., urethra, ureter, renal pelvis) transitional cell carcinoma of the urothelium.
2. Prior treatment with an FGFR inhibitor.
3. Known hypersensitivity to any study component including:
   Erdafitinib (or other drug excipients) or chemically related drugs, TAR-210 device constituent materials (e.g., thermoplastic polyurethane, silicone, nitinol),
   Urinary Placement Catheter materials (e.g., thermoplastic polyurethane).
4. Received pelvic radiotherapy≤6 months prior to the planned start of study treatment. If received pelvic radiotherapy>6 months prior to the start of study treatment, there must be no cystoscopic evidence of radiation cystitis.
5. Presence of any bladder or urethral anatomic feature that in the opinion of the investigator may prevent the safe placement, indwelling use, or removal of TAR-210.
6. Indwelling urinary catheter. Intermittent catheterization is acceptable.
7. Cystoscopic evidence of bladder perforation unless such perforation has resolved prior to dosing.
8. Bladder post-void residual volume (PVR)>350 mL after second voided urine.
9. History of clinically significant polyuria with recorded 24-hour urine volumes>4,000 mL.
10 Subjects with active bladder stones or history of bladder stones<6 months prior to the start of study treatment.
11. Active malignancies (i.e., progressing or requiring treatment change in the last 24 months) other than the disease being treated under study. Potential allowed exceptions include the following (others may be allowed with Sponsor approval).
   a. skin cancer (non-melanoma or melanoma) that is considered completely cured.
   non-invasive cervical cancer that is considered completely cured.
   adequately treated lobular carcinoma in situ (LCIS) and ductal CIS
   history of localized breast cancer and receiving antihormonal agents
   history of localized prostate cancer (NOMO) and receiving androgen deprivation therapy
   Localized prostate cancer (N0M0)
      with a Gleason score of6, treated within the last 24 months or untreated and under surveillance,
      with a Gleason score of3+4 that has been treated more than 6 months prior to full study Screening and considered to have a very low risk of recurrence,
      or history of localized prostate cancer and receiving androgen deprivation therapy and considered to have a very low risk of recurrence.
12. Current central serous retinopathy or retinal pigment epithelial detachment of any grade.
13. History of uncontrolled cardiovascular disease including:
   Any of the following within 3 months prior to the start of study treatment: unstable angina, myocardial infarction, ventricular arrhythmias or clinically significant atrial arrythmias (eg, atrial fibrillation with uncontrolled rate), cardiac arrest, or known congestive New York Heart Association Class III-IV heart failure (see https://www.heart.org/en/health-topics/heart-failure/what-is-heart-failure/classes-of-heart-failure. Accessed 16 Jul. 2021), cerebrovascular accident, or transient ischemic attack.
   Pulmonary embolism or other venous thromboembolism within 1 month prior to the planned start of study treatment.
14. Active or chronic hepatitis B or C infection according to the following criteria:
   Seropositive for hepatitis B: defined by a positive test for hepatitis B surface antigen [HBsAg]. Participants with resolved infection (ie, participants who are HbsAg negative with antibodies to total hepatitis B core antigen [anti-HBc] with or without the presence of hepatitis B surface antibody [anti-HBs]) must be screened using real-time polymerase chain reaction (RT-PCR) measurement of hepatitis B virus (HBV) DNA levels. Those who are RT-PCR positive will be excluded. Participants with anti-HBs positivity as the only serologic marker AND a known history of prior HBV vaccination do not need to be tested for HBV DNA by RT-PCR.
   Hepatitis C infection defined by a positive hepatitis C antibody (anti-HCV) test. Participants who test positive for anti-HCV are eligible if RNA viral load is undetectable (spontaneous recovery or after completing treatment for hepatitis C virus infection).
15. Major surgery within 4 weeks before Day 1 (TURBT is not considered major surgery)
16. Active bacterial, viral, fungal infection, including urinary tract infection*, requiring oral or systemic therapy within 7 days prior to Day 1
   *Urinary tract infection is defined as a symptomatic infection with a positive urine culture with a bacterial count of ≥10⁵ colony forming units (CFU)/mL in urine voided from women, or >10⁴ CFU/mL in urine voided from men, or in straight-catheter urine from women. Symptoms may include dysuria, urgency, frequency, and/or systemic symptoms such as fever, chills, elevated white blood cell, and/or abdominal/flank pain. Participants free from symptoms for 7 days with no culture evidence of >10⁵ CFUs may be eligible.
17. Toxicity from prior anticancer therapy has not resolved to baseline levels or to Grade≤1 (except alopecia, vitiligo, peripheral neuropathy, or endocrinopathies that are stable on hormone replacement, which may be Grade 2).
18. Known human immunodeficiency virus (HIV)-positive participants with 1 or more of the following:
   Not receiving highly active antiretroviral therapy (ART)
   Had a change in ART within 6 months of the start of screening
   Receiving ART that may interfere with study treatment
   CD4+ count<350 at screening
   Acquired immunodeficiency syndrome (AIDS)-defining opportunistic infection within 6 months of start of screening
   Not agreeing to start ART and be on ART>4 weeks prior to the start of study treatment. Only participants who have HIV viral load<400 copies/mL at the end of the 4-week period and agree to continue on ART, would be eligible (to ensure ART is tolerated and HIV controlled).

### Prior/Concurrent Clinical Study Experience

19. Received an investigational intervention (including investigational vaccines) or used an invasive investigational medical device within 28 days before the planned start of study treatment or is currently enrolled in an investigational study.
20. Prior anticancer therapy within 4 weeks before the planned start of study treatment. Exception: a single intravesical chemotherapy treatment immediately after TURBT is allowed.
21. Any condition for which, in the opinion of the investigator, participation would not be in the best interest of the participant (e.g., compromise the well-being) or that could prevent, limit, or confound the protocol-specified assessments.

### Example 10: Pharmacokinetics (PK) and Pharmacodynamics (PD) of Single-Dose Intravesical Erdafitinib Administration in Rats Bearing Orthotopic Bladder Tumors

The objective of Example 10 was to compare the PK and PD effects of localized bladder versus oral administration of erdafitinib in nude rats bearing human UM-UC-1 bladder xenografts. Animals were given a single oral dose (20 mg/kg erdafitinib in 10% weight per volume (w/v) HP-β-CD solution) or 1-hour intravesical instillation (6 mg/kg erdafitinib in 10% w/v HP-β-CD solution) of erdafitinib into the bladder. Extracellular signal-regulated kinase (ERK)1/2 phosphorylation was assessed as a PD marker for FGFR kinase inhibition in tumors at various time points post administration/installation. PK analysis of tumor and plasma samples was carried out at 2, 7, 48, and 120 hours after a single 6 mg/kg intravesical administration or a 20 mg/kg oral dose of erdafitinib. Additionally, a group of nude rats bearing subcutaneous (s.c.) tumors (UM-UC-1) were given erdafitinib orally and concentrations were measured in plasma and tumor at 2, 7, 48, and 120 hours post dose.

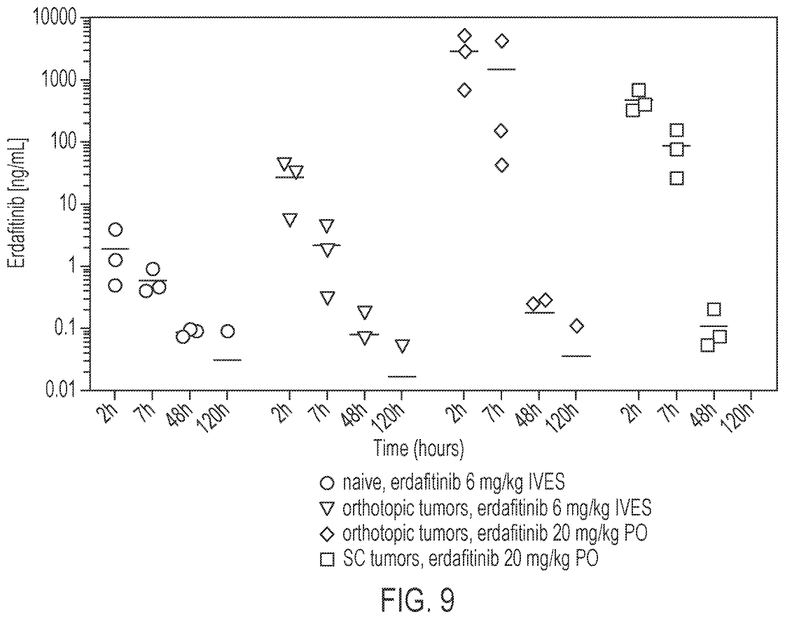

Intravesical administration resulted in mean erdafitinib exposure levels that were comparable to that of a 20 mg/kg oral dose (Table 6). Roughly 2-fold lower exposure levels were detected at 2 and 7 hours in subcutaneous (s.c.) tumors from rats dosed orally compared with orthotopic tumors from rats dosed orally, however it reflected the lower commensurate plasma exposures observed in the same group of rats (FIG. 9).

TABLE 6
Plasma and tumor exposure levels in naïve, UM-UC-1 orthotopic, or UM-UC-1 s.c.
tumor-bearing nude rats following a single oral or intravesical dose of erdafitinib.

| | | | | | | |
|---|---|---|---|---|---|---|
| Tumor | Dose | | | Plasma concentration^{a} | Tumor concentration^{b} | Tumor:plasma |
| site | (mg/kg) | Route | N | (ng/g) | (ng/g) | ratio^{c} |

Time post dose (hours)

| | | | | 2 | 7 | 2 | 7 | 2 | 7 |
|---|---|---|---|---|---|---|---|---|---|
| s.c. | 20 | p.o. | 3 | 468.7 | 86.4 | 1,242.3 | 453.7 | 2.7 | 5.2 |
| | | | | (187.7) | (64.8) | (321.7) | (60.1) | | |
| Bladder | 20 | p.o. | 3 | 2,850.7 | 1,454.8 | 3,321 | 983.3 | 1.2 | 0.7 |
| | | | | (2,189.1) | (2,352.1) | (1,625.3) | (661.8) | | |
| Bladder | 6 | IVES | 3 | 26.6 | 2.1 | 3,148.7 | 2,050.7 | 118.4 | 957.2 |
| | | | | (19.1) | (2.0) | (3,314.3) | (2,802.1) | | |
| Naïve | 6 | IVES | 3 | 1.9 | 0.6 | — | — | — | — |
| | | | | (1.8) | (0.3) | | | | |

HP-β-CD, hydroxypropyl β-cyclodextrin; IVES, intravesical (bladder instillation); LLOQ, lower limit of quantitation; PO or p.o., oral.
Values are mean with standard deviation in parentheses. Mean exposures for 48- and 120-hour timepoints were low (<35 ng/g). Erdafitinib was dosed as a 10% w/v HP-β-CD solution.
^{a}LLOQ serum = 0.05 ng/ml
^{b}LLOQ tumor = 2 ng/g
^{c}Average = tumor concentration (ng/g)/plasma concentration (ng/mL)

The effects of a single 20 mg/kg oral or 6 mg/kg intravesical dose of erdafitinib on ERK1/2 phosphorylation in orthotopic bladder UM-UC-1 tumors was assessed at various timepoints by capillary immunoblotting. Proteins from tumor sample lysates collected 2, 7, 48, and 120 hours after treatment with erdafitinib or vehicle were separated by capillary electrophoresis and probed with antibodies detecting phosphorylated (p)ERK1/2 and total ERK1/2. The signals for pERK1/2 were divided by the total ERK1/2 signal in the same sample and the mean of the pERK1/2 values for the corresponding vehicle-treated samples was set at a relative value of 1. At each timepoint, the pERK/ERK ratio for each tumor sample was divided by the mean pERK/ERK ratio derived from the corresponding control samples. There was 1 exception, as there were no vehicle-treated samples at the 120-hour timepoint, the pERK/ERK ratios for the erdafitinib-treated samples at the 120-hour timepoint were divided by the mean of the 48-hour vehicle-treated group.

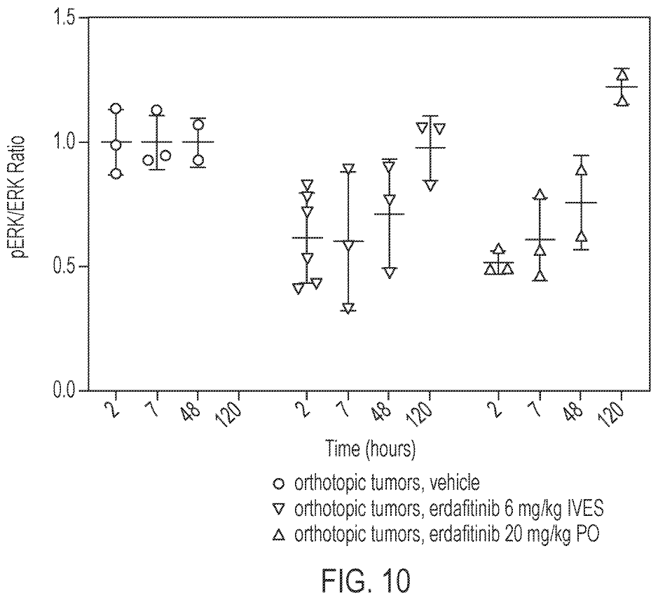

The 6 mg/kg intravesical and 20 mg/kg oral doses of erdafitinib both resulted in a statistically significant decrease in ERK1/2 phosphorylation in UM-UC-1 tumors at 2 hours post dosing (FIG. 10, Table 7). Although not statistically significant, pERK levels were also lower in the erdafitinib-treated tumors compared to vehicle treated tumors at 7 and 48 hours, whereas by 120 hours the levels of pERK1/2 were comparable to those of vehicle-treated rats.

TABLE 7
Levels of pERK/ERK in UM-UC1 orthotopic tumors following
a single oral or intravesical dose of erdafitinib.

| Time post | | | | | | |
|---|---|---|---|---|---|---|
| dose | Route of | Dose | | | | |
| (hours) | administration | (mg/kg) | N | pERK/ERK^{a} | | p-value^{b} |
| 2 | IVES | 0 | 3 | 1 | (0.13) | NA |
| 2 | IVES | 6 | 6 | 0.62 | (0.18) | 0.0102 |
| 2 | p.o. | 20 | 3 | 0.52 | (0.05) | 0.0063 |
| 7 | IVES | 0 | 3 | 1 | (0.11) | NA |
| 7 | IVES | 6 | 3 | 0.60 | (0.28) | 0.0841 |
| 7 | p.o. | 20 | 3 | 0.61 | (0.17) | 0.0893 |
| 48 | IVES | 0 | 2 | 1 | (0.10) | NA |
| 48 | IVES | 6 | 3 | 0.71 | (0.22) | 0.2682 |
| 48 | p.o. | 20 | 2 | 0.76 | (0.19) | 0.4065 |
| 120 | IVES | 6 | 3 | 0.98 | (0.13)^{c} | 0.9630 |
| 120 | p.o. | 20 | 2 | 1.22 | (0.07)^{c} | 0.1823 |

ANOVA, analysis of variance; ERK, extracellular signal-regulated kinase; IVES, intravesical; NA, not applicable; pERK, phosphorylated extracellular signal-regulated kinase; p.o., oral.
The “zero dose” animals were dosed with vehicle.
^{a}Ratio of pERK/ERK mean group values divided by the mean of the vehicle-treated group. Standard deviation between brackets.
^{b}One-way ANOVA, Dunnett's test for multiple comparison.
^{c}Values divided by mean of the 48-hour vehicle-treated group.

Overall, these data demonstrate that intravesical administration of erdafitinib provides adequate tumor PK/PD while dramatically reducing exposures in plasma, thereby decreasing potential for on-target, off-tumor toxicities compared to oral therapy.

### Example 11: Continuous Perfusion Studies in Orthotopic Bladder Cancer Model

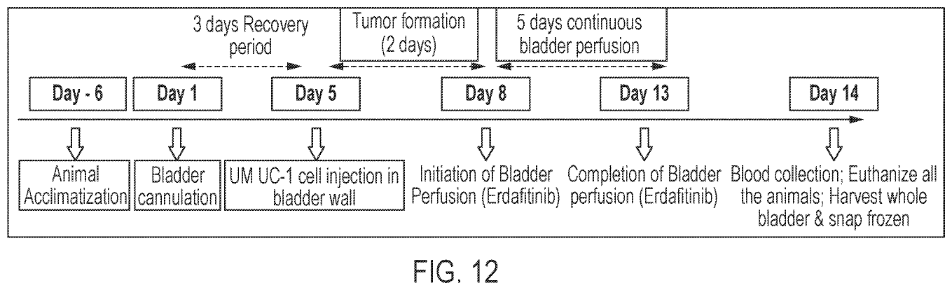

Perfusion studies: The bladders of the study animals were cannulated on Day 1 and the animals were allowed to recover for 3 days. On Day 5, UM-UC-1 cells (2×10⁶ cells) were injected into the lateral wall of each bladder. Following a 2-day tumor growth period, erdafitinib was perfused continuously for 5 days, followed by necropsy within 24 hours. Based on the in vitro results, target urine concentrations of 0.5, 1.0, and 5.0 μg/mL were used in the perfusion experiments. The study design is shown in FIG. 12. Body weight, daily urine production, and daily water consumption were recorded. At necropsy, a plasma sample, bladder photographs, and bladder weight measurements were recorded. Post necropsy total bladder weight, which is comprised of normal bladder tissue plus urothelial tumor, was used to determine the effect of erdafitinib on tumor growth.

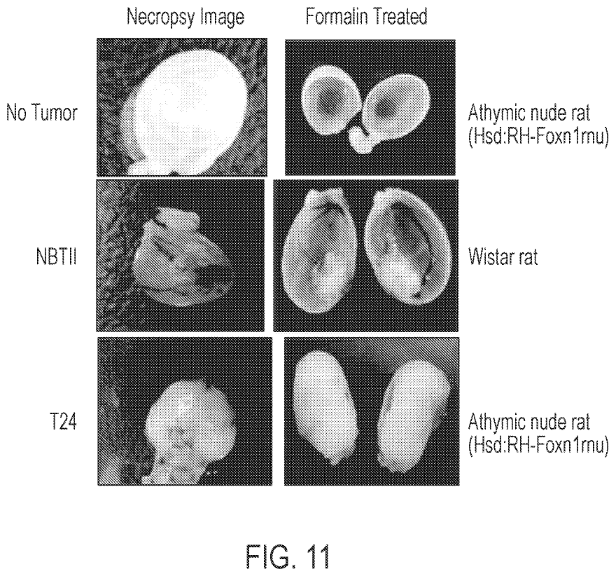

Human-derived tumor cells, when implanted into the bladder wall of athymic rats, grew rapidly. Within 7 days of implantation, the tumor occupied the majority of the urothelial surface and increased total bladder weight up to 7-fold (FIG. 11). Thus, total bladder weight is an accurate measure of drug response.

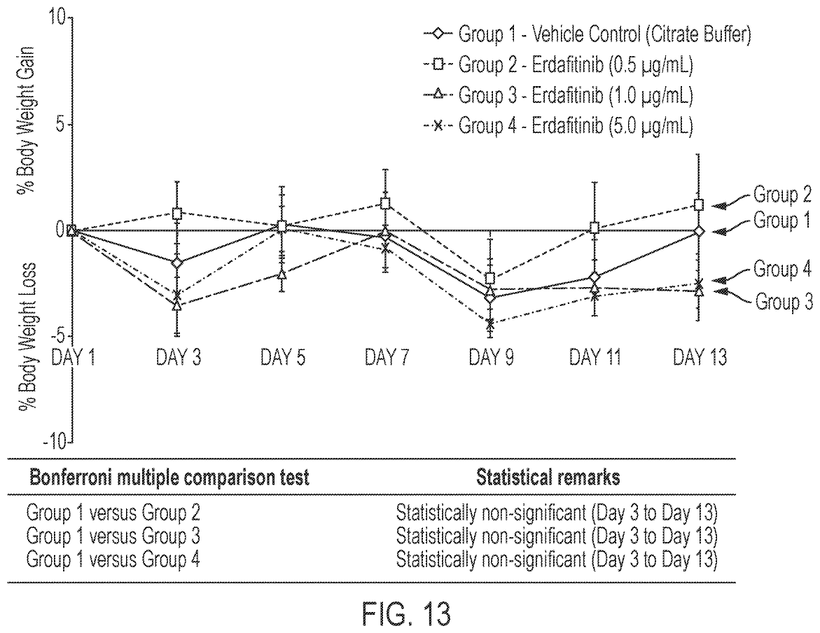

Five days of continuous erdafitinib perfusion at nominal urine concentrations of 0.5, 1.0, and 5.0 μg/mL was generally well tolerated. Body weight changes during the study are shown in FIG. 13. An initial small reduction in body weight, <5% (Day 1-3), was seen in most groups including vehicle control due to the impact of the bladder cannulation surgery. Minimal body weight changes were noted after intra-bladder tumor cell injection on Day 5. Transfer to metabolism cages and initiation of the bladder perfusions resulted in a second small body weight decrease unrelated to perfusate drug concentration. Based on cage side observations, no visible signs of abnormal behavior or clinical symptoms were observed in any of the treatment groups.

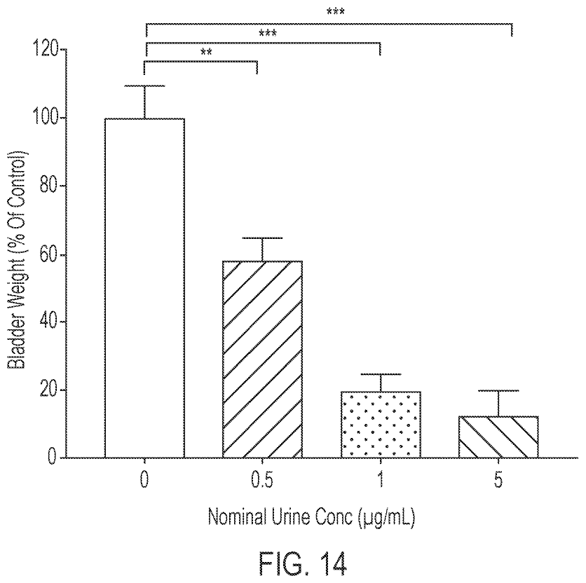

The percent reduction in relative tumor weight between the control group and the drug perfusion groups was determined as an initial measure of efficacy. Bladder tissue and tumor samples were also submitted for analysis of FGFR signaling activity by determining the phosphorylated fibroblast growth factor receptor substrate (FRS)2a levels and the pERK to ERK ratio. Additional urine, plasma, and bladder samples were collected to determine erdafitinib concentration using established liquid chromatography-tandem mass spectrometry (LC-MS/MS) methods. The mean bladder weight in animals receiving different erdafitinib concentrations is shown in FIG. 14. A significant dose-related decrease in bladder weight was observed in the erdafitinib treatment groups (**p<0.01 for erdafitinib at 0.5 μg/mL, ***p<0.001 for erdafitinib at 1.0 and at 5 μg/mL nominal urine concentrations) when compared to the vehicle control group

### Example 12: Dose-Response Evaluation of Erdafitinib in Bladder-Perfused Athymic Rats with RT-112 Implanted into the Bladder Wall

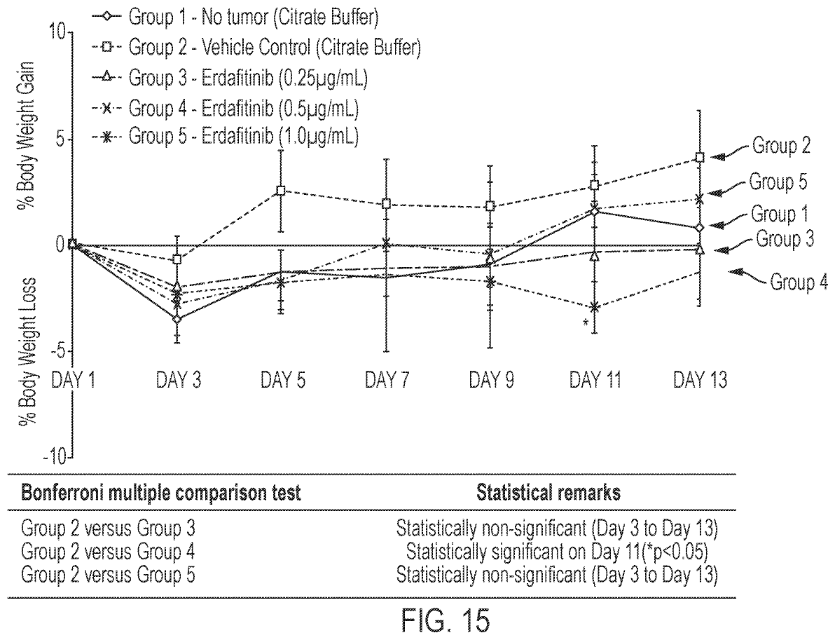

Perfusion studies: The experimental study design was the same as described in Example 11 (FIG. 12), except that the perfusions were continued until the time of necropsy on Day 14. Six days of continuous perfusion treatment with erdafitinib at the nominal urine concentrations of 0.25, 0.5, and 1.0 μg/mL was well tolerated during the experimental period. Body weight changes observed during the study are shown in FIG. 15. Mild body weight loss was observed during study conduct, with maximum mean values ranging up to −3% in the erdafitinib-treated group on Day 11.

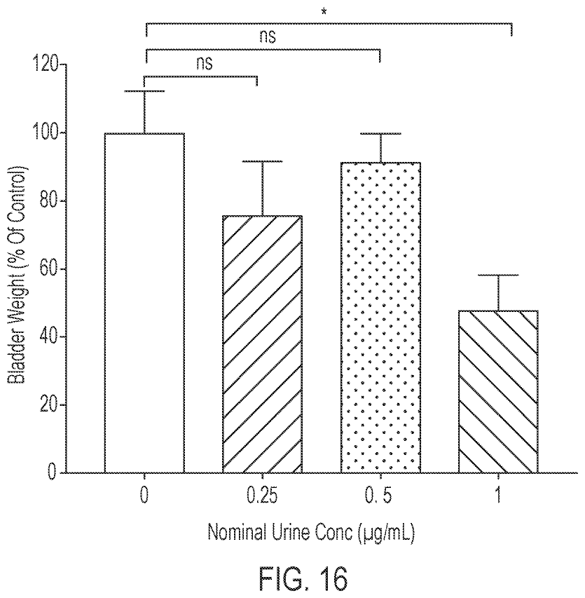

The effect of intravesical erdafitinib exposure on tumor growth as determined by changes in total bladder weight is shown in FIG. 16. Mean bladder weights trended lower with increasing erdafitinib concentration, but decreases were not statistically significant for the 0.25 and 0.5 μg/mL dose groups relative to vehicle control animals. A significant bladder weight reduction (*p<0.05) was observed in animals receiving the 1.0 μg/mL perfusate concentration compared to vehicle control.

Dose-response evaluation of erdafitinib (0.25-5 μg/mL) in bladder-perfused athymic rats with UM-UC-1 or RT-112 cell lines implanted into the bladder wall demonstrated that the dosing regimen of erdafitinib was generally well tolerated. Significant dose-dependent bladder weight decreases were observed in the erdafitinib treatment groups when compared to the vehicle control group, demonstrating the bladder perfusion of erdafitinib reduced tumor growth.

### Example 13: Intravesical Pharmacokinetic and Distribution Studies in Rats and Minipigs

Systemic and urinary bladder PK studies were conducted following single intravesical (bolus) administration of erdafitinib in solution (HP-β-CD) formulation to rats and minipigs. Additionally, bladder tissue was evaluated for gross and microscopy examination to determine whether there were any local effects of the drug or formulation in the study. The goal of Example 13 was to determine the feasibility of erdafitinib intravesical therapy with bladder installation of erdafitinib.

Single intravesical dose PK in rats: Systemic and urinary bladder PK of erdafitinib was determined in female Sprague-Dawley rats after intravesical administration of erdafitinib solution at 2, 6, and 18 mg per kg body weight. Rats were kept under anesthesia using isoflurane (2-4%), a catheter was introduced into the urethra to the bladder, and erdafitinib solution (10% w/v HP-β-CD in citrate buffer pH 5.5) was instilled to the rat bladder via this catheter. Solution formulations were prepared at various strengths in order to administer 0.5 mL volume to each rat bladder to dose 2, 6, and 18 mg/kg. Corresponding doses in nominal amount of drug were 0.5, 1.5, and 4.5 mg, respectively. After 1 hour of post-installation, rats were transferred to metabolic cages for collection of samples for PK determination. Blood samples were taken from the tail vein at 24, 48, 72, 96, and 168 hours after completing the 1-hour contact time of compound after dosing (3 rats per time point). At each blood sampling time point, bladder samples were taken from each rat for drug analysis. In addition, bladders collected at 96 hours in the 18 mg/kg dose (high-dose) group were microscopically evaluated. Urine collections were limited to initial 0-6 hours post dose from all rats.

In plasma, almost all samples were below the quantification limit (0.02 ng/mL) for the 2 and 6 mg/kg dose group. For the 18 mg/kg dose group, some measurable concentrations were observed at 24, 48, and 72 hours (between 0.0303 and 0.106 ng/mL), but at 96 and 168 hours all samples were below the quantification limit. In the bladder, concentrations could be measured up to 72 hours for the 2 and 6 mg/kg dose group, and up to 168 hours for the 18 mg/kg dose group. Concentrations were highest at the 24-hour time point and declined thereafter. No dose-linearity could be observed. Exposures were similar between the tested doses. The percentage of compound eliminated as unchanged drug in the urine (in the first 6 hours), amounted to 23.5%, 19%, and 50.3% for the 2, 6, and 18 mg/kg dose group.

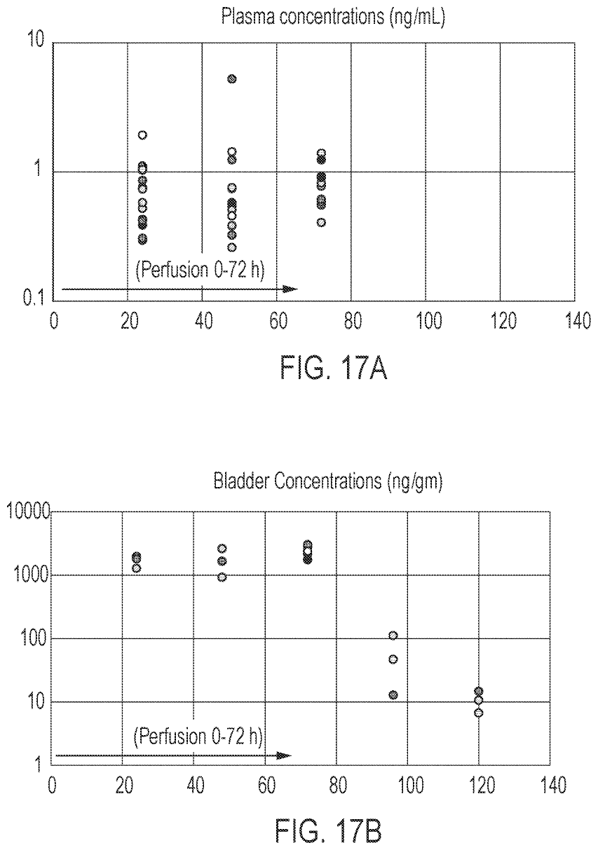

Systemic and bladder PK of continuous intravesical erdafitinib in rats: Systemic and bladder PK of erdafitinib was determined in female Sprague-Dawley rats after continuous intravesical infusion of an aqueous solution of erdafitinib. Rat bladders (5 groups of rats, n=3/group) were surgically catheterized under anesthesia and the catheter was externalized, tunneled subcutaneously, and connected to a vascular access harness (VAH) in the neck. Rats were transferred to individual metabolic cages with free access to food and water during the 1-week post-surgery recovery period. on the study day erdafitinib solution (0.1 mg/mL, 0.1 mL/hour, citrate buffer pH 5.5 containing 5% w/v HP-β-CD) was perfused through rat bladders via catheter for over 72 hours. The first group (n=3) was sacrificed at 24 hours post perfusion and 2 of the 4 remaining groups were sacrificed at 48 and 72 hours, post perfusion. Perfusion was stopped at 72 hours for the last 2 groups, and these groups were sacrificed at 96 and 120 hours to determine the drug elimination phase from bladder (Table 8). Plasma and bladder samples were collected at all time points. Urine was collected from the third group during the 48-72 hours perfusion period for drug analysis. Plasma concentrations in rats following 72-hour bladder perfusion of erdafitinib solution (0.1 mg/mL, 0.1 mL/hour, cumulative dose 0.72 mg) are shown in FIG. 17A. There were no levels detected (below quantification limit; 0.2 ng/mL) in plasma samples up to 120 hours (i.e., additional 48 hours) after stopping the perfusion at 72 hours. Bladder levels in rats following 72-hour bladder perfusion of erdafitinib solution (0.1 mg/mL, 0.1 mL/hour, cumulative dose 0.72 mg) are shown in FIG. 17B. Rat bladders perfused with 0.1 mg/mL erdafitinib solution did not show changes, and this formulation strength was considered tolerable. Average daily urine concentration was measured at about 10,000 ng/mL for urine collected during the perfusion interval of 48-72 hours.

TABLE 8
Plasma and bladder exposure following
bladder perfusion of erdafitinib.

| Parameter | Plasma | Bladder | Concentration | Plasma | Bladder |
|---|---|---|---|---|---|
| AUC_{0-24 h} | 8.09 | 20,200 | C_{24 h} (ng/mL) | 0.674 | 1,680 |
| (ng · h/mL) | | | | | |
| AUC_{0-48 h} | 27.2 | 61,100 | C_{48 h} (ng/mL) | 0.916 | 1,730 |
| (ng · h/mL) | | | | | |
| AUC_{0-72 h} | 48.1 | 111,000 | C_{72 h} (ng/mL) | 0.829 | 2,420^{a} |
| (ng · h/mL) | | | | | |
| AUC_{0-96 h} | 48.1 | 126,000 | C_{96 h} (ng/mL) | BQL | 56.9 |
| (ng · h/mL) | | | | | |
| AUC_{0-120 h} | 48.1 | 127,000 | C_{120 h} (ng/mL) | BQL | 10.7 |
| (ng · h/mL) | | | | | |

AUC₀₋ₓₕ, area under the plasma concentration − time curve from time of administration until x hours; Cₓₕ, plasma concentration at time x; NC, not calculated.
^{a}Average daily urine concentrations at this time point was about 10,000 ng/mL.

Results indicated marked bladder tissue uptake of erdafitinib and maintenance of high bladder levels with minimal systemic exposure upon continuous slow-rate perfusion of erdafitinib.

Systemic and bladder PK of continuous intravesical erdafitinib in pig: Systemic and bladder PK of erdafitinib was assessed in five female pigs (Domestic Yorkshire Crossbred swine) after continuous intravesical infusion of an aqueous solution of erdafitinib. On Day −7 a catheter was surgically placed in the bladder of each animal. The distal end of the catheter was anchored to a subcutaneous site and attached to a vascular access port (VAP). The port was secured and the animals were allowed to recover. Each animal was subsequently fitted with a portable infusion pump that was attached to the bladder catheter via the VAP. Dose formulations (22.5 μg/mL erdafitinib solution in 50 mM citrate buffer pH 6.0) were prepared every day and sterile filtered daily and analyzed to confirm the concentration. Dose formulation was perfused into the bladder at a constant rate of 12.5 mL/hour for 6 consecutive days for 2 animals and 8 consecutive days for 3 animals. All excreted urine was collected in 24-hour intervals through Day 6 or 8. Blood samples were collected daily on study Days 1 to 8. Bladder tissue samples were collected from each animal at necropsy. Samples obtained from all animals were analyzed for erdafitinib using qualified LC-MS/MS methods. Based on the formulation analysis on all days, the average daily administered dose for each animal ranged from 7.06 to 7.56 mg and overall mean dose was 7.3 mg/day. Based on the average bodyweight (pre dose), administered dose was 0.22 mg/kg/day.

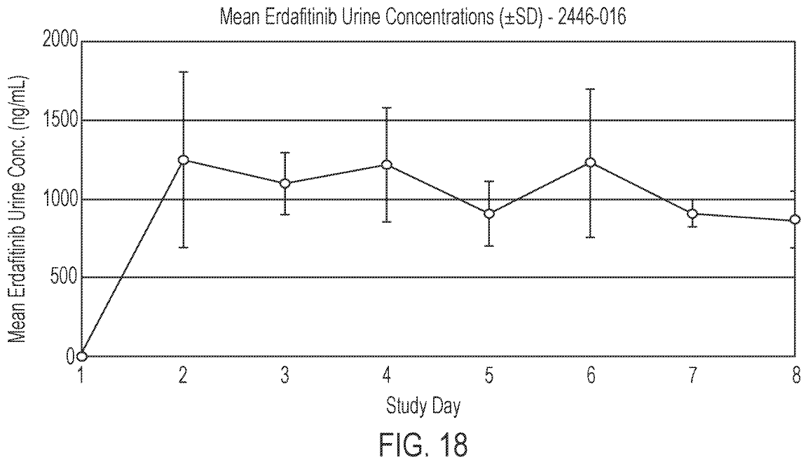

Mean (±SD) erdafitinib urine concentrations ranged from 1,255±554 to 873±179 ng/mL on Days 2 to 8 (FIG. 18). Over the 7-day period of this study, the average (±SD) daily urine output was 966±253 mL. Although inter- and intra-animal variation in daily urine production was observed, no significant trends in urine production were observed over the treatment period. Erdafitinib urinary recoveries were relatively consistent in all animals, averaging 910±812 to 1,135±760 ng on Days 2 to 8. Daily erdafitinib recoveries averaged 15.7% 5.67% of the average daily amount of erdafitinib administered.

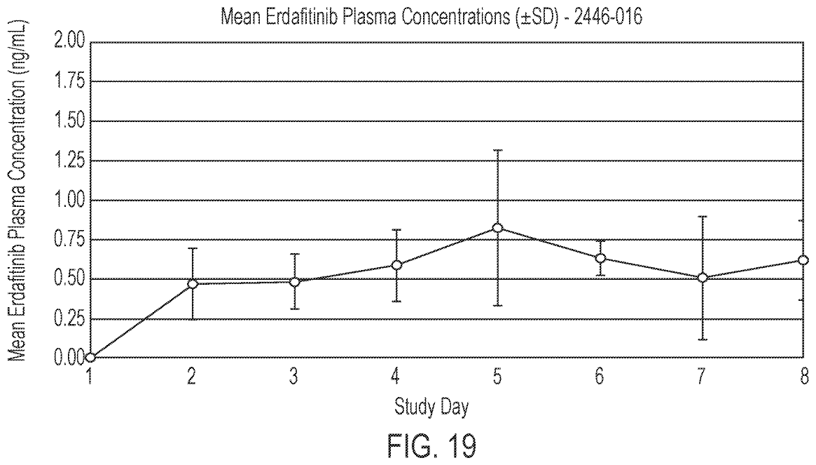

Erdafitinib plasma concentrations averaged (±SD) 0.622±0.250 to 0.828±0.487 ng/mL on Days 2 to 8 (FIG. 19).

Mean erdafitinib concentrations in full thickness bladder tissues were measured on Day 6 and Day 8 (end of perfusion) and the values ranged from 315 to 998 ng/g and 346 to 2,688 ng/g, respectivelyErdafitinib concentration was measured in urothelium and underlying tissue layers (i.e., muscle). These data suggest a >10-fold concentration of erdafitinib in the urothelial layer of the bladder when compared to the underlying tissue layers, suggestive that the drug was mainly retained in the urothelium. Mean bladder to urine concentrations were calculated for animals in 6-day and 8-day perfusion groups and the values were 0.60 and 2.33, respectively. Mean data are presented in Table 9.

TABLE 9
Erdafitinib bladder tissue concentrations in pig following
bladder perfusion of erdafitinib for 7 days.

| | | | | | | | Mean |
|---|---|---|---|---|---|---|---|
| | | | | | | | bladder |
| | | | | | | Mean | conc/mean |
| | Erdafitinib concentration (ng/g) | | | | | bladder | urine conc |
| | Bladder tissue | | | | Mean | conc/mean | ratio by |
| Bladder | | O'all | Urothel | Rem | urine conc | urine conc | perfusion |
| tissue | Mean | Mean | layer | layer | (ng/mL) | ratio | duration |
| Animal 1001, Day 8 | | | | | | | Day 8 |
| Dome | 2,620 | 1,913 | 4,520 | 147 | 991.9 | 1.93 | 2.33 |
| Lateral wall | 1,850 | | 3,290 | 198 | | | |
| Trigone | 1,270 | | 2,330 | 66.4 | | | |
| Animal 1002, Day 8 | | | | | | | Day 8 |
| Dome | 2,527 | 2,688 | 5,170 | 737 | 578.9 | 4.64 | |
| Lateral wall | 4,127 | | 11,600 | 382 | | | |
| Trigone | 1,411 | | 174 | 1,740 | | | |
| Animal 1003, Day 8 | | | | | | | Day 8 |
| Dome | 469 | 346 | 277 | 25.1 | 849.1 | 0.41 | |
| Lateral wall | 422 | | 612 | 37.4 | | | |
| Trigone | 145 | | 227 | 19.7 | | | |
| Animal 1004, Day 8 | | | | | | | Day 8 |
| Dome | 1,240 | 998 | 6,900 | 299 | 1,044 | 0.96 | 0.60 |
| Lateral wall | 1,193 | | 7,490 | 298 | | | |
| Trigone | 561 | | 1,390 | 118 | | | |
| Animal 1005, Day 8 | | | | | | | Day 8 |
| Dome | 334 | 315 | 1,420 | 125 | 1,268 | 0.25 | 1.30 |
| Lateral wall | 372 | | 2,170 | 111 | | | |
| Trigone | 238 | | 439 | 38.6 | | | |

conc, concentration; O'all, overall; Rem layer, bladder underlying tissue layer; Urothel layer, bladder urothelium.

### Example 14: Stability and Protein Binding Studies

Stability in urine: Urine was spiked with erdafitinib at 1, 3, and 5 μg/mL and incubated (in triplicates) at 37° C. for 6 hours as part of the equilibrium dialysis study. At the end of 6-hours post-dialysis incubation, drug was analyzed and the recovery was calculated against the spiked concentrations. The percent recovery of erdafitinib in the study was ranging from 89% to 98% in human urine, 90% to 95% in rat urine, and 92% to 93% in minipig urine, indicating that erdafitinib is stable in urine. These results suggest that erdafitinib will remain stable in urine in the bladder to provide exposure to tumor and bladder tissue.

### Example 15: Prototype Development

Based upon experiments, including animal studies, release rates of 1 mg/day, 2 mg/day 4 mg/day, and 6 mg/day were selected for further development. Designs enabling 30-day and 90-day use durations were evaluated. The 30-day designs were engineered to provide higher drug release rates that also exceeded the device 90-day payload capacity. The minimum target release rate was defined as the rate required to yield average erdafitinib urine concentrations of 1 μg/mL. Higher release rates were also evaluated to increase tumor exposures and to assess local tolerability and systemic exposure liabilities. Additional performance metrics included urine pH, urine volume, and urine composition independence.

A chemical gradient delivery approach was investigated to determine the optimal device release mechanism. Erdafitinib exhibits significant pH-dependent solubility over the normal urine pH range of 5.5 to 7. As a result, different drug formats and minitablet excipient combinations were evaluated to minimize the effect of urine pH and composition on system release rate.

A factorial design-based screen was first completed to evaluate the complete range of possible release rates and pH effects. Approximately 900 combinations of device polymers and erdafitinib drug formats were tested using powder packed, short core systems, which were 2-cm versions known to accurately scale to the full-length 15-cm design. The drug formats evaluated included erdafitinib free base, erdafitinib free base plus HP-β-CD and erdafitinib salts, e.g. HCl salt.

### Material Screening

In general, materials that are impermeable to erdafitinib, the API, are suitable for use as the base material in permeation systems. Materials that are permeable to the API are suitable for use as the stripe material in permeation systems. Platinum cured silicone, thermoplastic polyurethanes (TPUs), and ethylene vinyl acetate (EVA) materials were screened (Table 10). The test articles were filled with formulated API (powder or tablets), sealed, placed in foil pouches, and gamma irradiated (nominal 35 kGy). The systems were placed into simulated urine (pH 6.8), stored at 37° C., and sampled periodically. The amount of API in each sample was determined by high-performance liquid chromatography (HPLC) analysis.

TABLE 10
Materials screened.

| | Raw material | Test article | | |
|---|---|---|---|---|
| Material | manufacturer | form | Hardness | Description |
| Silicone | NuSil | Tube | 50A | High-consistency silicone elastomer, |
| | | | | platinum cured, |
| | | | | MED-4750 |
| TPU | Lubrizol | Tube | 70A | Carbothane ™ Aliphatic, PC-3575A |
| | | | 62A | Tecothane ™ Soft, AR-62A |
| | | | 42D | Tecophilic ™, HP-60D-35 |
| | | | 77A | Carbothane ™ Aromatic, AC-4075A |
| | | | 75A | Tecothane ™, TT-1074A |
| | | | 83A | Tecophilic ™, HP-93A-100 |
| | | | 72A | Tecoflex ™, EG-80A |
| EVA | 3M | Film | NA | Co Tran ™, 9712, 0.05 mm thick film |

EVA, ethylene vinyl acetate;
NA, not available;
TPU, thermoplastic polyurethane

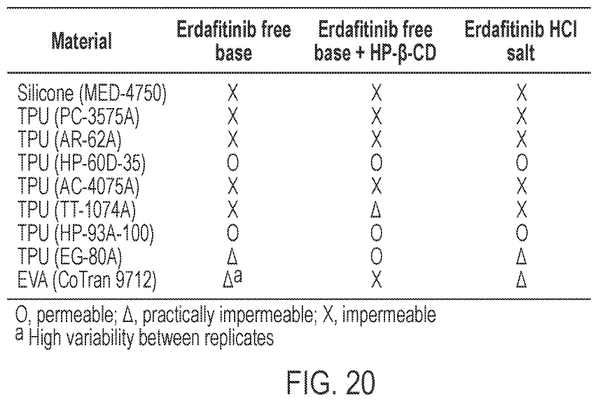

Permeability screening results are shown in FIG. 20. Materials that were suitable for use as the base material in a permeation system were impermeable or practically impermeable to the API such that the release rate would be too slow to achieve the target dose (e.g., TPU AR-62A, AR-75A, AC-4075A, and EG-80A). Materials that were suitable for use as the stripe material in a permeation system were permeable to the API. TPU materials HP-60D-35, HP-93A-100, or EG-80A were selected as possible stripe materials for further development of a permeation prototype.

### Permeation Systems

Release rates from the material screening studies were used to determine specifications for custom permeation tubing extrusions: stripe material, stripe angle (30°-180°), and wall thickness (0.2-0.41 mm). The inner diameter (ID) of the custom extrusions was set at 2.64 mm.

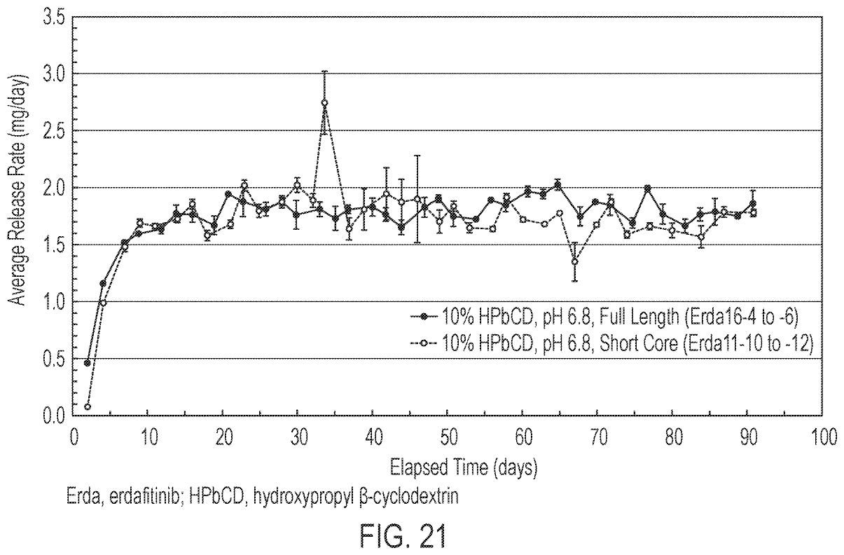

Short core systems enabled rapid screening of both drug and device constituents. FIG. 21 shows release rate from a full-length system for permeation systems.

The release rate increases as the stripe angle increases. Short core systems were assembled with the difference between the two device constituents being the stripe angle: 300 versus 90°.

### Simulated Urine pH Effect

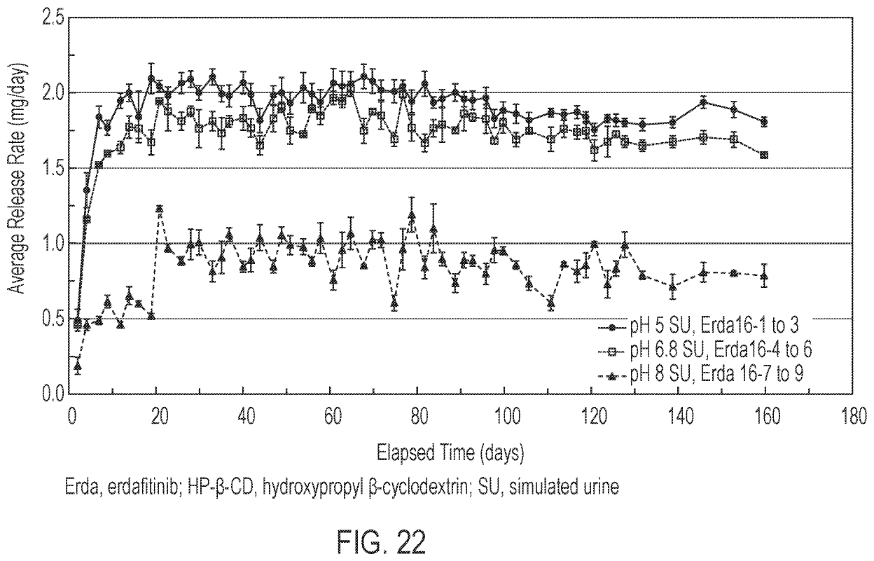

The pH range for normal human urine is in the 5.5 to 7 range, but some disease states can result in higher urine pH values (pH 8). Several permeation system prototypes exhibited similar release profiles in simulated urine at pH 5 and 6.8, and slightly lower release rates at pH 8. FIG. 22 shows that permeation prototypes with erdafitinib free base+HP-β-CD tablets (JNJ-42756493-1), with EG-80A as the stripe material, had similar release profiles in simulated urine at pH 5 and 6.8. FIG. 22 also shows that the release is zero-order for at least 160 days. Based on these results, it was determined that permeation prototype with erdafitinib free base+HP-β-CD tablets, an EG-80A stripe (450 to 180°), 0.2 mm wall thickness, and 15 cm drug core would produce a release profile of 1-4 mg FBE/day, based on stripe angle. The base material for this tubing could be either AR-75A-B20 or AC-4075A-B20.

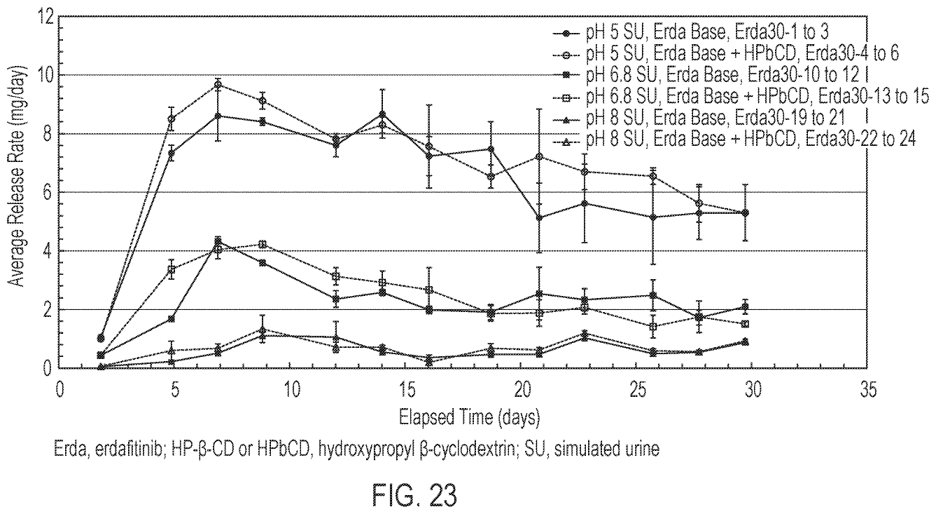

Additional short core prototype systems were tested to better understand the reduced pH effect. Short cores were assembled with the same device constituent: AR-62A base with 90° HP-60D-35 stripe, 2.64 mm ID, 0.41 mm wall thickness. The drug constituent in these systems was erdafitinib free base, with and without HP-β-CD. The drug constituent with HP-D-CD contained (w/w): 50% erdafitinib free base, 10% HP-β-CD, 25% Avicel PH101, 12.5% DCP, 1% meglumine, 0.5% Aerosil, and 1% magnesium stearate. The drug constituent without HP-D-CD contained: 50% erdafitinib free base, 20% MCC, 18.5% dicalcium phosphate, 8% Kollidon, 1% meglumine, 0.5% Aerosil, and 2% magnesium stearate. FIG. 23 shows that the addition of HP—β-CD to the erdafitinib free base formulation did not change the release profile with the HP-60D-35 stripe material and that the release profiles in simulated urine at pH 5 and 6.8 did not agree well for the erdafitinib free base tablets (with or without HP-β-CD) when used with the HP-60D-35 stripe material. This contrasts with the release profiles with erdafitinib free base+HP-β-CD tablets when used with the EG-80A stripe material (FIG. 22).

Permeation Systems with Erdafitinib Free Base

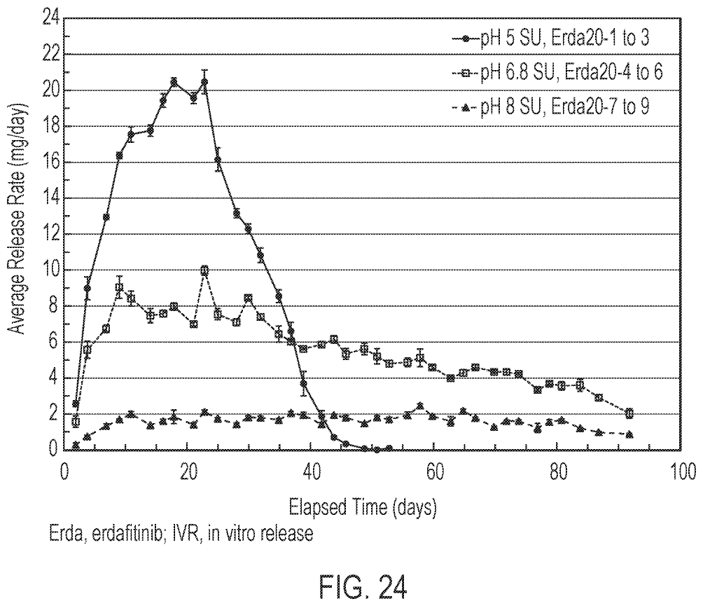

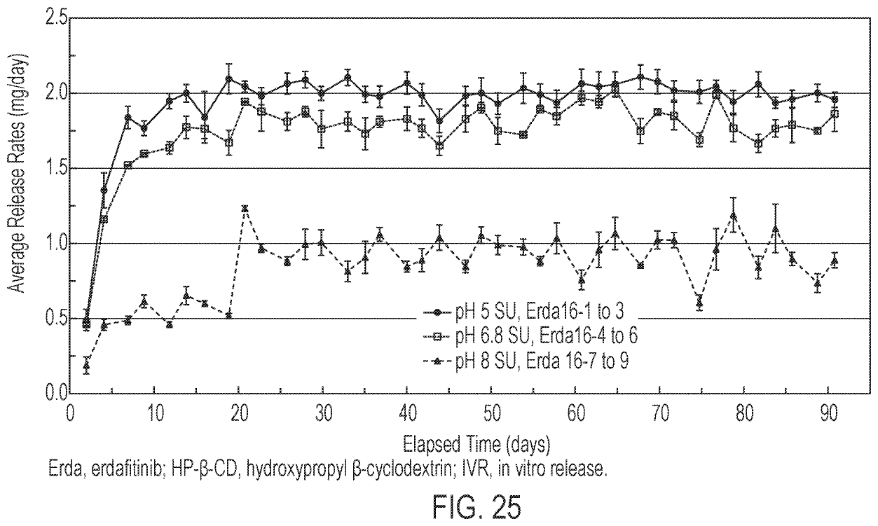

Two permeation system prototypes with erdafitinib free base were tested in minipigs (Prototype 1: Permeation (wireform), erdafitinib free base, tablets; Prototype 2: Permeation (wireform), erdafitinib free base+HP-β-CD (10% w/w), tablets). Drug and device constituents are described in Tables 11 and 12, respectively. In vitro release (IVR) testing was performed with both prototypes, using simulated urine at 3 different pH ranges as the release media: pH 5, 6.8, and 8 (n=3 systems tested in each media). The IVR profile for Prototype 1 showed some simulated urine pH dependency, with fastest release at pH 5 and slowest release at pH 8 (FIG. 24). The IVR profile for Prototype 2 showed zero-order release for 90 days, with good agreement in pH 5 and pH 6.8 simulated urine, and slightly slower release in pH 8 simulated urine (FIG. 25).

TABLE 11
Erdafitinib free base minitablet compositions (drug constituents).

| | Prototype 1 | Prototype 2 | |
|---|---|---|---|
| Component | % w/w | % w/w | Function |
| JNJ42756493-AAA | 50 | 50 | API |
| (Erdafitinib free base) | | | |
| HP-β-CD | NA | 10 | Solubilizer |
| Microcrystalline | 20 | 10 | Binder |
| cellulose (Avicel PH 105) | | | |
| Meglumine | 1 | 1 | Stabilizer |
| Dibasic calcium phosphate | 18.5 | 19 | Diluent |
| anhydrous (Emcompress AN) | | | |
| Vinyl pyrrolidone-vinyl | 8 | 8 | Binder |
| acetate (Kollidon VA64) | | | |
| Colloidal silicon dioxide | 0.5 | 0.50 | Glidant |
| (Aerosil 200) | | | |
| Magnesium stearate | 2.00 | 1.50 | Lubricant |
| (Ligamed MF-2V) | | | |
| Total | 100 | 100 | |

API, active pharmaceutical ingredient; HP-β-CD, hydroxypropyl β-cyclodextrin; NA, not applicable; w/w, weight per weight.

TABLE 12
Device constituent and final system parameters.

| | | |
|---|---|---|
| Parameter | Prototype 1 | Prototype 2 |

| | | |
|---|---|---|
| Tubing base material | EG-80A-B20 | AR-62A |
| Tu Tubing stripe material | HP-60D-35 | EG-80A |
| Stripe angle | 90° | 90° |

| | | | | |
|---|---|---|---|---|
| Drug core length | 15 | cm | 15 | cm |

| | | |
|---|---|---|
| Retentive feature | Wireform | Wireform |

| | | | | |
|---|---|---|---|---|
| Tablet mass | 1,054 | mg | 974 | mg |

| | | |
|---|---|---|
| API mass | 527 mg FBE | 487 mg FBE |

API active pharmaceutical ingredient;
FBE, free base equivalent;
ID, inner diameter

### Pharmacokinetic Evaluation in Minipigs

Permeation designs released erdafitinib at rates designed to provide the target urine concentration or greater. Depending upon the release rate, both first-order and zero-order release profiles were observed. The first-order designs demonstrated peak in vitro release rates up to 8 mg/day compared to zero-order systems (defined as release at a constant rate for at least 30 days) of up to 2 mg/day (>90-day duration). pH dependency was observed and was found to be dependent upon drug format. Erdafitinib free base and erdafitinib HCl salt both exhibited significant pH dependency with highest release rates observed at pH 5 compared to significantly reduced rates at pH 8. Inter- and intra-device release rate variance was lowest with the zero-order systems.

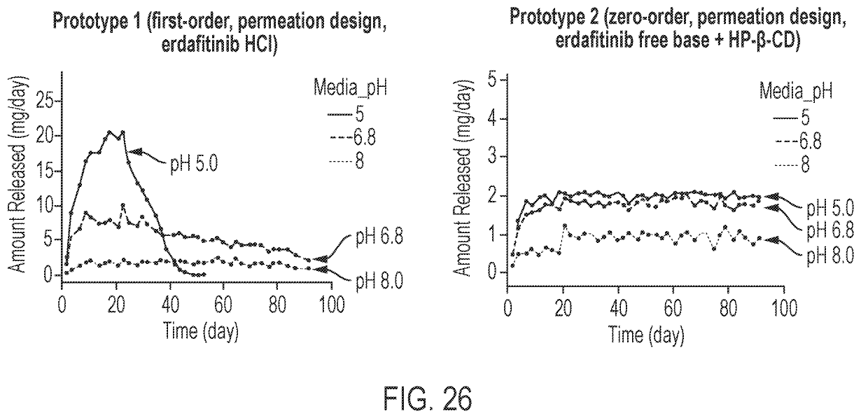

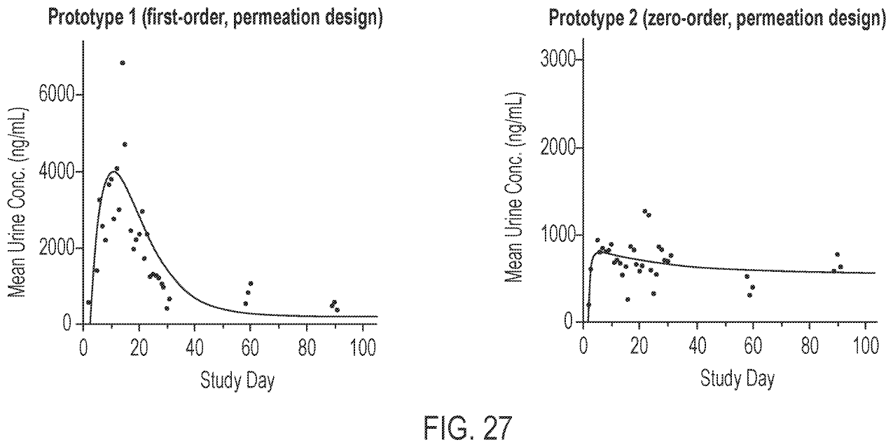

Based on the short core data, a series of full-length systems were developed and tested confirming the short core results. A subset of these were tested in minipigs to determine the in vivo release rate characteristics of the permeation designs. The in vivo results largely confirmed the in vitro findings. FIG. 26 summarizes the in vitro release characteristics of representative permeation systems selected for minipig testing. FIG. 27 summarizes the urine concentration versus time profiles of the same systems. Prototype 1 was a representative first-order design based on permeation tubing designs. Prototype 2 was a representative zero-order design based on permeation design.

Prototype 2 was selected for further development. Selection was based on demonstration of attaining at least 1 μg/mL average urine concentration, achievement of zero-order release over at least 90 days, and the ability to adjust the drug delivery rate up to 4 mg/day. Prototype 2 was further refined to increase release rate to 2 and 4 mg/day (pH 7), which was accomplished by increasing the surface area (as defined by stripe angle) of the permeation polymer co-extruded with the nonpermeable base polymer.

### Example 16: Device Development

### Formulation—Device Development

Formulation development was focused on erdafitinib free base+10% w/w HP-β-CD minitablet (Table 13).

TABLE 13
JNJ-42756493-1 (erdafitinib free base) minitablet composition.

| Component | % w/w | Function |
|---|---|---|
| JNJ-42756493-AAA | 50 | API |
| HP-β-CD | 10 | Solubilizer |
| Microcrystalline cellulose (Avicel PH 105) | 10 | Binder |
| Meglumine | 1 | Stabilizer |
| Dibasic calcium phosphate | 19 | Diluent |
| anhydrous (Emcompress AN) | | |
| Vinyl pyrrolidone-vinyl | 8 | Binder |
| acetate (Kollidon VA64) | | |
| Colloidal silicon dioxide (Aerosil 200) | 0.50 | Glidant |
| Magnesium stearate | 1.50 | Lubricant |
| (Ligamed MF-2V) | | |
| Total | 100 | |

API, active pharmaceutical ingredient; HP-β-CD, hydroxypropyl β-cyclodextrin; w/w, weight per weight.

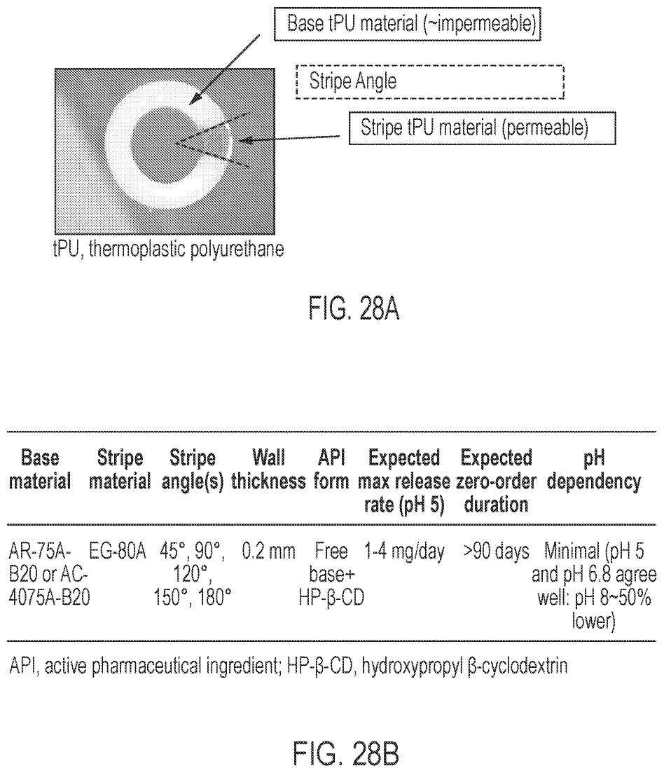

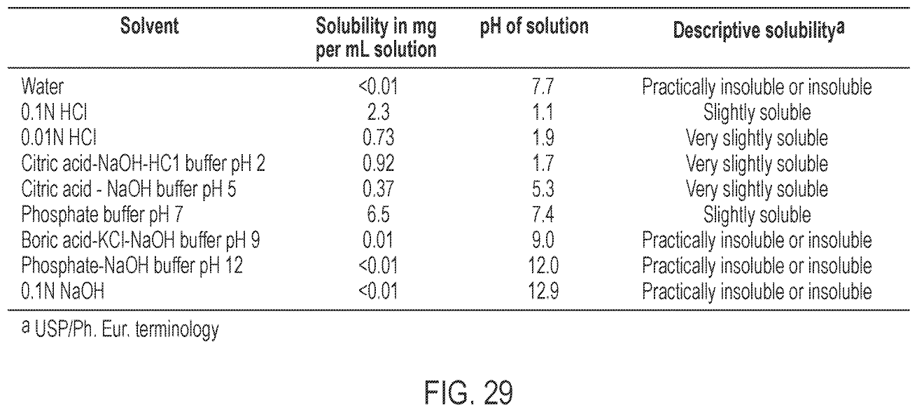

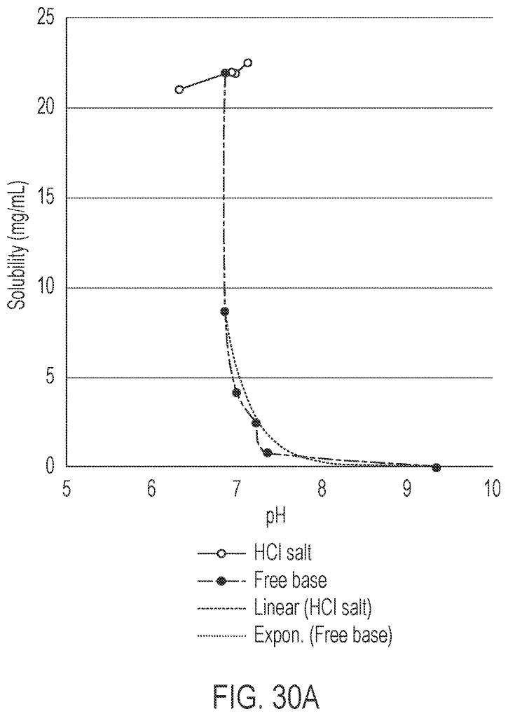

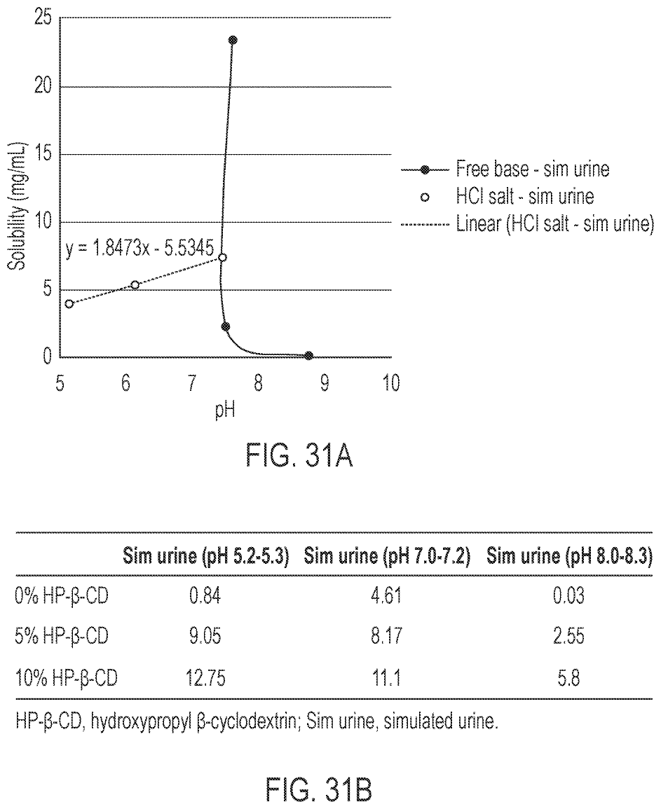

The permeation TPU system (diffusion driven, no orifice) was evaluated. Permeation design is shown in FIGS. 28A and 28B. Solubility of JNJ-42756493-AAA (free base) as a function of pH at 20° C. and 37° C. is shown in FIGS. 29 and 30A. Solubilities of JNJ-42756493-AAA (free base) and JNJ-42756493-AAC (HCl salt Form 1) as a function of pH at 20° C. and in simulated urine at 37° C. is shown in FIGS. 31A-31B. Varying the stripe angle of the permeable tubing controlled the release rate of the drug.

Many modifications and other implementations of the disclosure set forth herein will be apparent having the benefit of the teachings presented in the foregoing descriptions and the associated drawings. Therefore, it is to be understood that the disclosure is not to be limited to the specific implementations disclosed and that modifications and other implementations are intended to be included within the scope of the appended claims. Although specific terms are employed herein, they are used in a generic and descriptive sense only and not for purposes of limitation.

In the descriptions provided herein, the terms “includes,” “is,” “containing,” “having,” and “comprises” are used in an open-ended fashion, and thus should be interpreted to mean “including, but not limited to.” When methods, compositions, or apparatuses are claimed or described in terms of “comprising” various steps or components, then the methods, composition, or apparatuses can also “consist essentially of” or “consist of” the various steps or components, unless stated otherwise. In the case of chemical compounds or compositions, the use of “consisting essentially of” means that only those further components not materially affecting the essential characteristics of the specified compound or composition may be present.

We claim:

1. A solid pharmaceutical composition comprising:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of at least 45 wt % of the solid pharmaceutical composition; and
(b) at least one pharmaceutical excipient.

2. The solid pharmaceutical composition of claim 1, wherein the at least one pharmaceutical excipient comprises a solubilizer, a binder, a diluent (filler), a wetting agent, a disintegrant, a glidant, a lubricant, a formaldehyde scavenger, or any combination thereof.

3. A process for making a solid pharmaceutical composition comprising:
(a) preparing an intragranular solid composition comprising or consisting essentially of:
(i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine); and
(ii) at least one intragranular pharmaceutical excipient;
(b) combining the intragranular solid composition with at least one extragranular pharmaceutical excipient to form a blend; and
(c) tableting the blend to form the solid pharmaceutical composition, wherein the erdafitinib free base is present in a concentration of at least 45 wt % of the solid pharmaceutical composition.

4. The process for making a solid pharmaceutical composition according to claim 3, wherein at least one intragranular pharmaceutical excipient and at least one extragranular pharmaceutical excipient comprise or are selected from at least one common (mutually occurring) pharmaceutical excipient.

5. The process for making a solid pharmaceutical composition according to claim 4, wherein the at least one intragranular excipient and the at least one extragranular pharmaceutical excipient do not comprise a common pharmaceutical excipient.

6. The process for making a solid pharmaceutical composition according to any of claims 3-5, wherein the intragranular solid composition is prepared by a roller compaction process or a fluid bed granulation process.

7. The process for making a solid pharmaceutical composition according to any of claims 3-6, wherein the at least one extragranular pharmaceutical excipient comprises microcrystalline cellulose and vinylpyrrolidone-vinyl acetate copolymer, in particular in a weight ratio of 50:50.

8. The process for making a solid pharmaceutical composition according to any of claims 3-7, wherein:
(a) the intragranular solid composition comprises a solubilizer, at least one binder, and a first quantity of a lubricant;
(b) the extragranular pharmaceutical excipients comprise a diluent, a glidant, and a second quantity of a lubricant; and
(c) the intragranular solid composition is prepared by a roller compaction process.

9. The process for making a solid pharmaceutical composition according to claim 8, wherein:
the solubilizer is hydroxypropyl-beta-cyclodextrin;
the binder is a combination of microcrystalline cellulose and vinylpyrrolidone-vinyl acetate copolymer;
the lubricant is magnesium stearate;
the diluent is anhydrous dibasic calcium phosphate; and
the glidant is colloidal silicon dioxide.

10. The process for making a solid pharmaceutical composition according to any of claims 3-7, wherein:
(a) the intragranular solid composition comprises a solubilizer, a diluent, and a disintegrant;
(b) the extragranular pharmaceutical excipients comprise at least one binder and a lubricant; and
(c) the intragranular solid composition is prepared by a fluid bed granulation process.

11. The process for making a solid pharmaceutical composition according to claim 10, wherein:
the solubilizer comprises hydroxypropyl-beta-cyclodextrin;
the diluent comprises microcrystalline cellulose;
the disintegrant comprises hydroxypropyl methylcellulose;
the at least one binder comprises a combination of microcrystalline cellulose and vinylpyrrolidone-vinyl acetate copolymer; and
the lubricant comprises magnesium stearate.

12. The solid pharmaceutical composition or the process for making a solid pharmaceutical composition according to any of claims 1-11, wherein the erdafitinib free base is present in the solid pharmaceutical composition in a concentration of from 45 wt % to 55 wt %, from 47 wt % to 53 wt %, or about 50 wt % and/or wherein the at least one extragranular excipient comprises microcrystalline cellulose and vinylpyrrolidone-vinyl acetate copolymer, in particular in a weight ratio of 50:50.

13. The solid pharmaceutical composition or the process for making a solid pharmaceutical composition according to any of claims 1-12, wherein the solid pharmaceutical composition further comprises a formaldehyde scavenger selected from the group consisting of meglumine, glycine, alanine, serine, threonine, cysteine, valine, leucine, isoleucine, methionine, phenylalanine, tyrosine, aspartic acid, glutamic acid, arginine, lysine, ornithine, taurine, histidine, aspartame, proline, tryptophan, citrulline, pyrrolysine, asparagine, glutamine, tris(hydroxymethyl)aminomethane, conjugates thereof, pharmaceutically acceptable salts thereof, or any combination thereof.

14. The solid pharmaceutical composition or the process for making a solid pharmaceutical composition according to claim 13, wherein the formaldehyde scavenger is meglumine.

15. The solid pharmaceutical composition or the process for making a solid pharmaceutical composition according to claim 13 or 14, wherein the formaldehyde scavenger is present in the solid pharmaceutical composition in a concentration of from 0.01 wt % to 5 wt %, from 0.05 wt % to 3 wt %, from 0.1 wt % to 2 wt %, from 0.5 wt % to 1.5 wt %, or about 1 wt %.

16. The solid pharmaceutical composition or the process for making a solid pharmaceutical composition according to any of claims 1-15, wherein the at least one pharmaceutical excipient, the at least one intragranular pharmaceutical excipient or the at least one extragranular pharmaceutical excipient comprises a solubilizer selected from the group consisting of hydroxypropyl-beta-cyclodextrin, hydroxypropyl-gamma-cyclodextrin, sulfobutyl ether-beta-cyclodextrin sodium salt, hydroxypropyl methylcellulose acetate succinate, hydroxypropyl methylcellulose E5 (HPMC-E5), or any combination thereof.

17. The solid pharmaceutical composition or the process for making a solid pharmaceutical composition according to claim 16, wherein the solubilizer is hydroxypropyl-beta-cyclodextrin.

18. The solid pharmaceutical composition or the process for making a solid pharmaceutical composition according to claim 17, wherein the total concentration of the solubilizer in the solid pharmaceutical composition is from 1 wt % to 20 wt %, from 5 wt % to 15 wt %, from 7 wt % to 12 wt %, or about 10 wt %.

19. The solid pharmaceutical composition or the process for making a solid pharmaceutical composition according to any of claims 1-18, wherein the at least one pharmaceutical excipient, the at least one intragranular pharmaceutical excipient or the at least one extragranular pharmaceutical excipient comprises or further comprises at least one binder selected from the group consisting of polyvinylpyrrolidone (PVP), poly(vinyl acetate) (PVA), vinylpyrrolidone-vinyl acetate copolymer, polyethylene oxide (PEO), polypropylene oxide (PPO), an ethylene glycol-propylene glycol copolymer, a poloxamer, hydroxypropyl cellulose (HPC), hydroxypropyl methylcellulose (HPMC), microcrystalline cellulose, silicified microcrystalline cellulose, and combinations thereof.

20. The solid pharmaceutical composition or the process for making a solid pharmaceutical composition according to claim 19, wherein the total concentration of the at least one binder in the solid pharmaceutical composition is from 5 wt % to 30 wt %, from 10 wt % to 25 wt %, from 12 wt % to 22 wt %, or from 14 wt % to 19 wt %.

21. The solid pharmaceutical composition or the process for making a solid pharmaceutical composition according to any of claims 1-20, wherein the at least one pharmaceutical excipient, the at least one intragranular pharmaceutical excipient or the at least one extragranular pharmaceutical excipient comprises or further comprises a wetting agent.

22. The solid pharmaceutical composition or the process for making a solid pharmaceutical composition according to claim 21, wherein the wetting agent comprises sodium lauryl sulfate, sodium stearyl fumarate, polysorbate 80, docusate sodium, or any combination thereof.

23. The solid pharmaceutical composition or the process for making a solid pharmaceutical composition according to any of claims 21-22, wherein the total concentration of the wetting agent in the solid pharmaceutical composition is from 0.01 wt % to 2.5 wt %, from 0.05 wt % to 1.0 wt %, or from 0.1 wt % to 0.5 wt %.

24. The solid pharmaceutical composition or the process for making a solid pharmaceutical composition according to any of claims 1-23, wherein the at least one pharmaceutical excipient, the at least one intragranular pharmaceutical excipient or the at least one extragranular pharmaceutical excipient comprises or further comprises a disintegrant.

25. The solid pharmaceutical composition or the process for making a solid pharmaceutical composition according to claim 24, wherein the disintegrant comprises hydroxypropyl methylcellulose, low-substituted hydroxypropylcellulose, crospovidone (crosslinked polyvinylpyrrolidone), croscarmellose sodium (cross-linked sodium carboxymethylcellulose), sodium starch glycolate, or any combination thereof.

26. The solid pharmaceutical composition or the process for making a solid pharmaceutical composition according to any of claims 24-25, wherein the total concentration of the disintegrant in the solid pharmaceutical composition is from 0.1 wt % to 3 wt %, from 0.5 wt % to 2.5 wt %, from 1 wt % to 2 wt %, or about 1.5 wt %.

27. The solid pharmaceutical composition or the process for making a solid pharmaceutical composition according to any of claims 1-24, wherein the at least one pharmaceutical excipient, the at least one intragranular pharmaceutical excipient or the at least one extragranular pharmaceutical excipient comprises or further comprises a diluent.

28. The solid pharmaceutical composition or the process for making a solid pharmaceutical composition according to claim 27, wherein the diluent comprises lactose (lactose monohydrate), dextrin, mannitol, sorbitol, starch, microcrystalline cellulose, dibasic calcium phosphate, anhydrous dibasic calcium phosphate, calcium carbonate, sucrose, or any combination thereof.

29. The solid pharmaceutical composition or the process for making a solid pharmaceutical composition according to any of claims 27-28, wherein the total concentration of the diluent in the solid pharmaceutical composition is from 12 wt % to 30 wt %, from 15 wt % to 25 wt %, or from 18 wt % to 22 wt %.

30. The solid pharmaceutical composition or the process for making a solid pharmaceutical composition according to any of claims 1-29, wherein the at least one pharmaceutical excipient, the at least one intragranular pharmaceutical excipient or the at least one extragranular pharmaceutical excipient comprises or further comprises a glidant.

31. The solid pharmaceutical composition or the process for making a solid pharmaceutical composition according to claim 30, wherein the glidant comprises colloidal silicon dioxide, colloidal anhydrous silicon dioxide, talc, or any combination thereof.

32. The solid pharmaceutical composition or the process for making a solid pharmaceutical composition according to any of claims 30-31, wherein the total concentration of the glidant in the solid pharmaceutical composition is from 0.01 wt % to 5 wt %, 0.05 wt % to 3 wt %, 0.1 wt % to 1 wt %, or about 0.5 wt %.

33. The solid pharmaceutical composition or the process for making a solid pharmaceutical composition according to any of claims 1-32, wherein the at least one pharmaceutical excipient, the at least one intragranular pharmaceutical excipient or the at least one extragranular pharmaceutical excipient comprises or further comprises a lubricant.

34. The solid pharmaceutical composition or the process for making a solid pharmaceutical composition according to claim 33, wherein the lubricant comprises magnesium stearate, stearic acid, magnesium silicate, aluminum silicate, isopropyl myristate, sodium oleate, sodium stearoyl lactate, sodium stearoyl fumarate, titanium dioxide, or combinations thereof.

35. The solid pharmaceutical composition or the process for making a solid pharmaceutical composition according to any of claims 33-34, wherein the total concentration of the lubricant in the solid pharmaceutical composition is from 0.05 wt % to 5 wt %, 0.1 wt % to 3 wt %, 1 wt % to 2 wt %, or about 1.5 wt %.

36. The solid pharmaceutical composition or the process for making a solid pharmaceutical composition according to any of claims 1-35, wherein the solid pharmaceutical composition is a mini-tablet.

37. The solid pharmaceutical composition or the process for making a solid pharmaceutical composition according to claim 36, wherein the mini-tablet is in the form of a solid cylinder having a cylindrical axis, a cylindrical side face, circular end faces perpendicular to the cylindrical axis, a diameter across the circular end faces, and a length along the cylindrical side face.

38. The solid pharmaceutical composition or the process for making a solid pharmaceutical composition according to claim 37, wherein length of the mini-tablet exceeds the diameter of the mini-tablet to provide the mini-tablet with an aspect ratio (length:diameter) of greater than 1:1.

39. The solid pharmaceutical composition or the process for making a solid pharmaceutical composition according to any of claims 37-38, wherein the mini-tablet has a diameter of from 1.0 mm to 3.2 mm, or from 1.5 mm to 3.1 mm.

40. A solid pharmaceutical composition consisting essentially of:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin in a concentration of 10 wt % of the solid pharmaceutical composition;
(c) meglumine in a concentration of 1 wt % of the solid pharmaceutical composition;
(d) microcrystalline cellulose in a concentration of 17.5 wt % of the solid pharmaceutical composition;
(e) silicified microcrystalline cellulose in a concentration of 10.75 wt % of the solid pharmaceutical composition;
(f) vinylpyrrolidone-vinyl acetate copolymer in a concentration of 7.5 wt % of the solid pharmaceutical composition;
(g) colloidal silicon dioxide in a concentration of 0.25 wt % of the solid pharmaceutical composition;
(h) hydroxypropyl methylcellulose in a concentration of 1.5 wt % of the solid pharmaceutical composition; and
(i) magnesium stearate in a concentration of 1.5 wt % of the solid pharmaceutical composition; or
a solid pharmaceutical composition comprising:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin;
(c) meglumine;
(d) microcrystalline cellulose;
(e) silicified microcrystalline cellulose;
(f) vinylpyrrolidone-vinyl acetate copolymer;
(g) colloidal silicon dioxide;
(h) hydroxypropyl methylcellulose; and
(i) magnesium stearate.

41. A solid pharmaceutical composition comprising:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin in a concentration of 10 wt % of the solid pharmaceutical composition;
(c) microcrystalline cellulose in a concentration of 17.5 wt % of the solid pharmaceutical composition;
(d) silicified microcrystalline cellulose in a concentration of 11.75 wt % of the solid pharmaceutical composition;
(e) vinylpyrrolidone-vinyl acetate copolymer in a concentration of 7.5 wt % of the solid pharmaceutical composition;
(f) colloidal silicon dioxide in a concentration of 0.25 wt % of the solid pharmaceutical composition;
(g) hydroxypropyl methylcellulose in a concentration of 1.5 wt % of the solid pharmaceutical composition; and
(h) magnesium stearate in a concentration of 1.5 wt % of the solid pharmaceutical composition; or
a solid pharmaceutical composition comprising:
(a) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(b) hydroxypropyl-beta-cyclodextrin;
(c) microcrystalline cellulose;
(d) silicified microcrystalline cellulose;
(e) vinylpyrrolidone-vinyl acetate copolymer;
(f) colloidal silicon dioxide;
(g) hydroxypropyl methylcellulose; and
(h) magnesium stearate.

42. A process for making a solid pharmaceutical composition comprising:
(a) preparing an intragranular solid composition by a fluid bed granulation process, the intragranular solid composition consisting essentially of:
(i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(ii) hydroxypropyl-beta-cyclodextrin in a concentration of 10 wt % of the solid pharmaceutical composition;
(iii) meglumine in a concentration of 1 wt % of the solid pharmaceutical composition;
(iv) microcrystalline cellulose in a concentration of 10 wt % of the solid pharmaceutical composition; and
(v) hydroxypropyl methylcellulose in a concentration of 1.5 wt % of the solid pharmaceutical composition;
(b) combining the intragranular solid composition with extragranular components to form a blend, wherein the extragranular components consist essentially of:
(i) microcrystalline cellulose in a concentration of 7.5 wt % of the solid pharmaceutical composition; and
(ii) vinylpyrrolidone-vinyl acetate copolymer in a concentration of 7.5 wt % of the solid pharmaceutical composition;
(iii) silicified microcrystalline cellulose in a concentration of 10.75 wt % of the solid pharmaceutical composition;
(iv) colloidal silicon dioxide in a concentration of 0.25 wt % of the solid pharmaceutical composition; and
(iv) magnesium stearate in a concentration of 1.5 wt % of the solid pharmaceutical composition; and
(c) tableting the blend to form of a solid pharmaceutical composition in the form of mini-tablets.

43. A process for making a solid pharmaceutical composition comprising:
(a) preparing an intragranular solid composition by a fluid bed granulation process, the intragranular solid composition consisting essentially of:
(i) erdafitinib free base (N-(3,5-dimethoxyphenyl)-N′-(1-methylethyl)-N-[3-(1-methyl-1H-pyrazol-4-yl)quinoxalin-6-yl]ethane-1,2-diamine) in a concentration of 50 wt % of the solid pharmaceutical composition;
(ii) hydroxypropyl-beta-cyclodextrin in a concentration of 10 wt % of the solid pharmaceutical composition;
(iii) microcrystalline cellulose in a concentration of 10 wt % of the solid pharmaceutical composition; and
(iv) hydroxypropyl methylcellulose in a concentration of 1.5 wt % of the solid pharmaceutical composition;
(b) combining the intragranular solid composition with extragranular components to form a blend, wherein the extragranular components consist essentially of:
(i) microcrystalline cellulose in a concentration of 7.5 wt % of the solid pharmaceutical composition; and
(ii) vinylpyrrolidone-vinyl acetate copolymer in a concentration of 7.5 wt % of the solid pharmaceutical composition;
(iii) silicified microcrystalline cellulose in a concentration of 11.75 wt % of the solid pharmaceutical composition;
(iv) colloidal silicon dioxide in a concentration of 0.25 wt % of the solid pharmaceutical composition; and
(iv) magnesium stearate in a concentration of 1.5 wt % of the solid pharmaceutical composition; and
(c) tableting the blend to form of a solid pharmaceutical composition in the form of mini-tablets.

44. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, wherein the first material comprises a polycarbonate-based aromatic thermoplastic polyurethane and the second material comprises an aliphatic polyether-based thermoplastic polyurethane; and
a drug formulation disposed in the closed drug reservoir lumen, the drug formulation comprising a drug,
wherein (i) the second wall structure, or both the first wall structure and the second wall structure, are permeable to water, and (ii) the first wall structure is impermeable to the drug and the second wall structure is permeable to the drug, such that the drug is releasable in vivo by diffusion through the second material forming the second wall structure.

45. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, wherein the first material comprises an aromatic polyester hydrocarbon-based thermoplastic polyurethane and the second material comprises an aliphatic polyether-based thermoplastic polyurethane; and
a drug formulation disposed in the closed drug reservoir lumen, the drug formulation comprising a drug,
wherein (i) the second wall structure, or both the first wall structure and the second wall structure, are permeable to water, and (ii) the first wall structure is impermeable to the drug and the second wall structure is permeable to the drug, such that the drug is releasable in vivo by diffusion through the second wall structure.

46. The drug delivery system of claim 44 or 45, wherein the second wall structure forms a longitudinal strip extending along the length of the tube.

47. The drug delivery system of any one of claims 44-46, wherein the drug delivery system is configured to release the drug over a period of 2 days to 6 months.

48. The drug delivery system of any one of claims 44-47, wherein the two interface edges are disposed at an arc angle of from 15 degrees to 270 degrees of a circumference of the tube in a cross section normal to a longitudinal axis of the tube.

49. The drug delivery system of any one of claims 44-48, wherein the drug is erdafitinib.

50. The drug delivery system of claim 49, wherein the drug delivery system is configured to release the erdafitinib at an average rate of 1 mg/day to 10 mg/day.

51. The drug delivery system of claim 50, wherein the two interface edges are disposed at an arc angle of 45 degrees to 90 degrees of a circumference of the tube in a cross section normal to a longitudinal axis of the tube.

52. The drug delivery system of claim 50, wherein the two interface edges are disposed at an arc angle of 150 degrees to 270 degrees of a circumference of the tube in a cross section normal to a longitudinal axis of the tube.

53. The drug delivery system of claim 50, wherein the system is configured to release the erdafitinib at an average rate of 2 mg/day.

54. The drug delivery system of claim 53, wherein the two interface edges are disposed at an arc angle of about 90 degrees of a circumference of the tube in a cross section normal to a longitudinal axis of the tube.

55. The drug delivery system of claim 50, wherein the system is configured to release the erdafitinib at an average rate of 4 mg/day.

56. The drug delivery system of claim 55, wherein the two interface edges are disposed at an arc angle of about 180 degrees of a circumference of the tube in a cross section normal to a longitudinal axis of the tube.

57. The drug delivery system of any one of claims 44-56, wherein the system comprises 500 mg of the erdafitinib.

58. The drug delivery system of any one of claims 44-57, wherein a release profile of the drug is substantially independent of pH over a pH range of 5 to 7.

59. The drug delivery system of any one of claims 44-58, wherein the second wall structure comprises less than 50 percent of a cross sectional area of the tube, in a cross section normal to the longitudinal axis of the tube.

60. The system of any one of claims 44-59, wherein the second wall structure comprises less than 25 percent of a cross sectional area of the tube, in a cross section normal to the longitudinal axis of the tube.

61. The system of any one of claims 44-60, wherein the tube has a substantially constant thickness over its circumference.

62. The drug delivery system of any one of claims 44-61, further comprising a pair of end plugs and/or an adhesive material that seal the ends of the tube.

63. The drug delivery system of any one of claims 44-62, wherein the first and second wall structures are integrally formed.

64. The drug delivery system of any one of claims 44-63, wherein the system is elastically deformable between a relatively straightened deployment shape suited for insertion through the urethra of a patient and into the patient's bladder and a retention shape suited to retain the system within the bladder.

65. The drug delivery system of any one of claims 44-64, wherein the system is elastically deformable and comprises overlapping curls formed by the tube, and the tube has two opposing free ends, which are directed away from one another when the system is in a low-profile deployment shape and which are directed toward one another when the system is in a relatively expanded retention shape.

66. The drug delivery system of any one of claims 44-65, wherein the system is elastically deformable and has a bi-oval retention shape, and the tube has two opposing free ends which lie within an outer boundary of the bi-oval retention shape.

67. The drug delivery system of any one of claims 44-66, further comprising a retention frame lumen.

68. The drug delivery system of any one of claims 44-67, wherein the first material has a Shore durometer value from 70A to 80A.

69. The drug delivery system of any one of claims 44-68, wherein the drug formulation comprises the solid pharmaceutical composition of any one of claims 1, 2 and 12-41.

70. The drug delivery system of any one of claims 44-69, wherein the drug formulation is in the form of a plurality of mini-tablets serially arranged in the drug lumen.

71. The drug delivery system of claim 70, wherein the plurality of mini-tablets comprise the mini-tablets of any one of claims 37-39.

72. A drug delivery system, comprising:
a housing defining a drug reservoir lumen bounded by a first wall structure formed of a first material and a second wall structure formed of a second material, wherein the first material comprises a polycarbonate-based aromatic thermoplastic polyurethane and the second material comprises an aliphatic polyether-based thermoplastic polyurethane; and
a drug formulation disposed in the drug reservoir lumen, the drug formulation comprising erdafitinib,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second material forming the second wall structure.

73. The drug delivery system of claim 72, wherein the first and second wall structures are adjacent one another at two interface edges and together form a tube, and (i) the drug delivery system is configured to release the erdafitinib at an average rate of 2 mg/day and the two interface edges are disposed at an arc angle of about 90 degrees of a circumference of the tube in a cross section normal to a longitudinal axis of the tube, (ii) the drug delivery system is configured to release the erdafitinib at an average rate of 4 mg/day and the two interface edges are disposed at an arc angle of about 180 degrees of a circumference of the tube in a cross section normal to a longitudinal axis of the tube, or (iii) the drug delivery system is configured to release the erdafitinib at an average rate of 6 mg/day and the two interface edges are disposed at an arc angle of 240 degrees.

74. The drug delivery system of any one of claims 72-73, wherein the system is elastically deformable and comprises overlapping curls formed by the tube, and the tube has two opposing free ends, which are directed away from one another when the system is in a low-profile deployment shape and which are directed toward one another when the system is in a relatively expanded retention shape.

75. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, with the second wall structure forming a longitudinal strip extending along the length of the tube, wherein the first material comprises a polycarbonate-based aromatic thermoplastic polyurethane and the second material comprises an aliphatic polyether-based thermoplastic polyurethane; and
a drug formulation disposed in the closed drug reservoir lumen, the drug formulation comprising the solid pharmaceutical composition of any one of claims 1, 2 and 12-41,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second material forming the second wall structure,
wherein the drug delivery system is configured to release a therapeutically effective amount of the erdafitinib at a substantially zero order release rate over at least 3 days, and
wherein (i) the drug delivery system is configured to release the erdafitinib at an average rate of 2 mg/day and the two interface edges are disposed at an arc angle of about 90 degrees of a circumference of the tube in a cross section normal to a longitudinal axis of the tube, (ii) the drug delivery system is configured to release the erdafitinib at an average rate of 4 mg/day and the two interface edges are disposed at an arc angle of about 180 degrees of a circumference of the tube in a cross section normal to a longitudinal axis of the tube, or (iii) the drug delivery system is configured to release the erdafitinib at an average rate of 6 mg/day and the two interface edges are disposed at an arc angle of 240 degrees.

76. A drug delivery system, comprising:
a housing defining a closed drug reservoir lumen bounded by a first wall structure formed of a first material and a second wall structure formed of a second material, the first and second wall structures being adjacent one another at two interface edges and together forming a tube defining the closed drug reservoir lumen, with the second wall structure forming a longitudinal strip extending along the length of the tube, wherein the first material comprises an aromatic polyester hydrocarbon-based thermoplastic polyurethane and the second material comprises an aliphatic polyether-based thermoplastic polyurethane; and
a drug formulation disposed in the closed drug reservoir lumen, the drug formulation comprising the solid pharmaceutical composition of any one of claims 1, 2 and 12-41,
wherein (i) the second wall structure, or both the first wall structure and the second wall structure, are permeable to water, and (ii) the first wall structure is impermeable to the erdafitinib and the second wall structure is permeable to the erdafitinib, such that the erdafitinib is releasable in vivo by diffusion through the second wall structure,
wherein the drug delivery system is configured to release a therapeutically effective amount of the erdafitinib at a substantially zero order release rate over at least 3 days, and
wherein (i) the drug delivery system is configured to release the erdafitinib at an average rate of 2 mg/day and the two interface edges are disposed at an arc angle of about 90 degrees of a circumference of the tube in a cross section normal to a longitudinal axis of the tube, or (ii) the drug delivery system is configured to release the erdafitinib at an average rate of 4 mg/day and the two interface edges are disposed at an arc angle of about 180 degrees of a circumference of the tube in a cross section normal to a longitudinal axis of the tube.

77. A method of treatment of bladder cancer, comprising locally delivering erdafitinib into the bladder of a patient in need thereof, in an amount effective for the treatment of bladder cancer.

78. The method of claim 77, wherein the erdafitinib is in the form of the solid pharmaceutical composition of any one of claims 1, 2 or 12-41.

79. A method of intravesical administration of erdafitinib, comprising:
deploying an intravesical system into the bladder of a patient, the intravesical system comprising the solid pharmaceutical composition of any one of claims 1, 2 or 12-41; and
releasing the erdafitinib from the system.

80. A method of treating bladder cancer comprising deploying an intravesical system into the bladder of a patient, the intravesical system comprising the solid pharmaceutical composition of any one of claims 1, 2 or 12-41.

81. A method of treating bladder cancer in a patient comprising delivering erdafitinib to the bladder deploying to the bladder of the patient, the drug delivery system of any one of claims 44-76.

82. The drug delivery system of any one of claims 70-76, wherein the drug delivery device comprises 44-46 of erdafitinib minitablets.

83. The drug delivery system of any of claims 70-76 or 82, wherein the erdafitinib minitablets comprises the solid pharmaceutical formulation of claim 40.

84. The drug delivery device of any of claims 70-76 or 82, wherein the erdafitinib minitablets comprises the solid pharmaceutical formulation of claim 41.

85. The drug delivery system of any one of claims 44-76 or 82-84, wherein the first material comprises AC-4075A-B20 or AR-75A and the second material comprises EG-80A.

86. The drug delivery system of any one of claims 44-76 or 82-85, wherein the first material has a shore hardness of about 78A; specific gravity of about 1.38; ultimate tensile (psi) of about 8300; ultimate elongation (%) of about 400 (D412); tensile modulus (psi) of about 560 at 100% elongation, about 1300 at 200% elongation, and about 3400 at 300% elongation (ASTM D412); flexural modulus (psi) of about 1800; Vicat temperature (° C.) of about 55; and/or mold shrinkage (in/in) (1″×0.25″×6″ bar) of about 0.011 (ASTM D955).

87. The delivery system of any one of claims 44-76 or 82-86, wherein the second material has a shore hardness of about 72A; specific gravity of about 1.04; flexural modulus (psi) of 1,000; ultimate tensile (psi) of about 5,800; ultimate elongation (%) of about 660 (D412); tensile modulus (psi) of about 300 at 100% elongation, about 500 at 200% elongation, and about 800 at 300% elongation (ASTM D412); and mold shrinkage (in/in) of about 0.008-0.0012.

88. The delivery system of any one of claims 44-76, 82-85 or 87, wherein the first material has a shore hardness of about 79A; a specific gravity of about 1.03; ultimate tensile (psi) of about 2000; ultimate elongation (%) of about 530; tensile modulus (psi) of about 730 at 100% elongation, about 1000 at 200% elongation, and about 1300 at 300% elongation; flexural modulus (psi) of about 2500 (ASTM 790); Vicat softening point (° C.) of about 75; and mold shrinkage (in/in) (1″×0.25″×6″ bar) of about 0.08.
